(19) United States
(12) Patent Application Publication
(10) Pub. No.: US 2012/0165339 A1
(43) Pub. Date: Jun. 28, 2012

(54) CYCLOPROPANE DERIVATIVES

(75) Inventors: Taro Terauchi, Tsukuba (JP); Ayumi Takemura, Tsukuba (JP); Yuji Kazuta, Tsukuba (JP); Fumihiro Ozaki, Tsukuba (JP); Takashi Doko, Tokyo (JP); Yu Yoshida, Tsukuba (JP); Toshiaki Tanaka, Tsukuba (JP); Keiichi Sorimachi, Tsukuba (JP); Yoshimitsu Naoe, Tsukuba (JP); Carsten Beuckmann, Tsukuba (JP)

(73) Assignee: EISAI R&D MANAGEMENT CO., LTD., Tokyo (JP)

(21) Appl. No.: 13/330,972

(22) Filed: Dec. 20, 2011

Related U.S. Application Data
(60) Provisional application No. 61/426,049, filed on Dec. 22, 2010.

(30) Foreign Application Priority Data
Dec. 22, 2010 (JP) 2010-285724

(51) Int. Cl. A61K 31/501; A61K 31/444; A61K 31/443; A61K 31/4439; A61K 31/44; A61K 31/4196; A61K 31/427; A61K 31/428; A61K 31/42; A61K 31/4184; A61K 31/415; A61K 31/165; C07D 237/16; C07D 413/12; C07D 401/12; C07D 405/12; C07D 213/75; C07D 285/08; C07D 417/12; C07D 275/03; C07D 261/14; C07D 249/08; C07D 231/56; C07D 231/18; C07C 233/60; A61P 25/20; A61K 31/50

(52) U.S. Cl. 514/252.03; 514/247; 514/335; 514/337; 514/338; 514/340; 514/352; 514/361; 514/371; 514/372; 514/380; 514/383; 514/394; 514/407; 514/622; 544/238; 544/240; 546/261; 546/269.1; 546/272.1; 546/272.4; 546/275.7; 546/284.1; 546/309; 548/128; 548/195; 548/214; 548/246; 548/265.8; 548/361.1; 548/370.7; 564/174

(57) ABSTRACT

A cyclopropane derivative represented by the following formula (I) or a pharmaceutically acceptable salt thereof has orexin receptor inhibitory action, and thus, is extremely useful as an agent for preventing or treating sleep disorder or dyssomnia caused by orexin, including insomnia as a typical example:

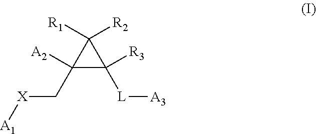

wherein A₁, A₂ and A₃ each independently represent an aryl group, a heterocyclyl group or the like, R₁, R₂ and R₃ each independently represent a hydrogen atom, a C₁₋₆ alkyl group or the like, X represents an oxygen atom or the like, and L represents a bond or the like.

### CROSS REFERENCE TO RELATED APPLICATIONS

The present application claims priority from U.S. Provisional Application No. 61/426,049 filed Dec. 22, 2010 and Japanese Patent Application No. 2010-285724 filed Dec. 22, 2010, all of the contents of which are incorporated herein by reference.

### BACKGROUND OF THE INVENTION

(1) Field of the Invention

The present invention relates to a cyclopropane derivative having orexin receptor antagonism or a pharmaceutically acceptable salt thereof, and a medicinal use thereof. The present invention also relates to a pharmaceutical composition comprising the same as an active ingredient.

(2) Description of related art

Orexin-A (OX-A, consisting of 33 amino acid peptides) and orexin-B (OX-B, consisting of 28 amino acid peptides), two types of intracerebral neuropeptides, which are expressed by neurons localized at the hypothalamus in the brain, have been discovered (JP 10-229887 A, Sakurai T. et al., Cell, 1998, 92, 573-585) as endogenous ligands of G protein-coupled receptors mainly existing in the brain, namely, orexin receptors (International Publication No. WO1996/34877, JP 10-327888 A, JP 10-327889 A, JP 11-178588 A). It has been known that such orexin receptors include two subtypes, namely, an OX₁ receptor (OX1) as a type 1 subtype and an OX₂ receptor (OX2) as a type 2 subtype. OX1 binds OX-A more selectively than OX-B, and OX2 is able to bind OX-A as well as OX-B. Orexin has been found to stimulate the food consumption of rats, and thus, it has been suggested that orexin would play a physiological role as a mediator in a central feedback mechanism for controlling feeding behavior (Sakurai T. et al., Cell, 1998, 92, 573-585). On the other hand, it has been observed that orexins control sleep-wake conditions. Thus, it is considered that orexins will potentially lead to new therapies for narcolepsy, as well as for insomnia and other sleep disorders (Chemelli R. M. et al., Cell, 1999, 98, 437-451). In addition, it has been suggested that orexin signals in the ventral tegmental area regarding neural plasticity associated with opioid dependence and nicotine dependence play an important role in vivo (S. L. Borgland et al. Neuron, 2006, 49, 589-601; C. J. Winrow et al. Neuropharmacology, 2010, 58, 185-194). It has been also reported that OX2 was selectively inhibited to alleviate ethanol dependence in experiment using rats (J. R. Shoblock et al., Psychopharmacology, 2011, 215, 191-203). Moreover, it has been reported that corticotropin-releasing factor (CRF), which involved in depression and anxiety disorder, is involved in orexin-induced behaviors in rats, and that orexin may play an important role in some stress reactions (T. Ida et al., Biochemical and Biophysical Research Communications, 2000, 270, 318-323).

Orexin receptors are found in the mammalian brain and may have numerous implications in pathologies such as depression; dysphoria; anxiety; addictions, obsessive compulsive disorder; affective neurosis; depressive neurosis; anxiety neurosis; dysthymic disorder; mood disorder; sexual dysfunction; psychosexual dysfunction; sex disorder; schizophrenia; manic depression; delirium; dementia; severe mental retardation and dyskinesias such as Huntington's disease and Tourette syndrome; eating disorders; sleep disorders; cardiovascular diseases, diabetes; appetite/taste disorders; vomiting/nausea; asthma; Parkinson's disease; Cushing's syndrome/disease; basophil adenoma; prolactinoma; hyperprolactinemia; hypopituitarism; hypophysis tumour/adenoma; hypothalamic diseases; inflammatory bowel disease; gastric dyskinesia; gastric ulcers; Froehlich's syndrome; hypophysis diseases, hypothalamic hypogonadism; Kallman's syndrome (anosmia, hyposmia); functional or psychogenic amenorrhea; hypopituitarism; hypothalamic hypothyroidism; hypothalamic-adrenal dysfunction; idiopathic hyperprolactinemia; hypothalamic disorders of growth hormone deficiency; idiopathic growth deficiency; dwarfism; gigantism; acromegaly; disturbed biological and circadian rhythms; sleep disturbances associated with diseases such as neurological disorders, neuropathic pain and restless leg syndrome; heart and lung diseases, acute and congestive heart failure; hypotension; hypertension; urinary retention; osteoporosis; angina pectoris; myocardial infarction; ischemic or haemorrhagic stroke; subarachnoid haemorrhage; ulcers; allergies; benign prostatic hypertrophy; chronic renal failure; renal disease; impaired glucose tolerance; migraine; hyperalgesia; pain; enhanced or exaggerated sensitivity to pain such as hyperalgesia, causalgia, and allodynia; acute pain; burn pain; atypical facial pain; neuropathic pain; back pain; complex regional pain syndrome I and II; arthritic pain; sports injury pain; pain related to infection e.g. HIV, post-chemotherapy pain; post-stroke pain; post-operative pain; neuralgia; conditions associated with visceral pain such as irritable bowel syndrome, migraine and angina; urinary bladder incontinence e.g. urge incontinence; tolerance to narcotics or withdrawal from narcotics; sleep apnea; narcolepsy; insomnia; parasomnia; and neurodegenerative disorders including nosological entities such as disinhibition-dementia-parkinsonism-amyotrophy complex; pallido-ponto-nigral degeneration epilepsy; seizure disorders and other diseases related to general orexin system dysfunction.

(2R)-2-{(1S)-6,7-dimethoxy-1-[2-(4-trifluoromethyl-phenyl)ethyl]-3,4-dihydro-1H-isoquinolin-2-yl}-N-methyl-2-phenylacetamide (ACT-078573; almorexant), a compound that functions as an orexin receptor antagonist, had been clinically developed as a therapeutic agent for insomnia (International Publication No. WO2005/118548). This compound causes a decrease in wakefulness in rats, which is characterized by decreased functions of awakening and spontaneous locomotor activity, and it dose-dependently increases both rapid eye movement (REM) sleep time and non-REM sleep time, and this compound, when administered to normal humans, exhibits dose-dependently a reduction of sleep latency, sleep efficacy and extension of total sleep time (F. Jenck et al., Nature Medicine 2007, 13, 150-155). There is also an article reporting that the compound, when administered to patients with insomnia, exhibits improvement of sleep efficacy, shortness of sleep latency, increase of REM sleep and improvement of REM sleep ratio (G. Dorffner et al., European Neuropsychopharmacology, Vol. 20, Supplement, 3, 2007, S252-S253). Furthermore, it has also been described that this compound improves the memory function of model rats (International Publication No. WO2007/105177), and that the compound is effective for posttraumatic stress disorder (International Publication No. WO2009/047723). On the other hand, 5-chloro-2-{(5R)-5-methyl-4-[5-methyl-2-(2H-1,2,3-triazol-2-yl)benzoyl]-1,4-diazepan-1-yl}-1,3-benzoxazole (MK-4305; suvorexant, International Publication No. WO2008/06999) and MK-6096, which have dual orexin antagonisms against OX1 and OX2, have been clinically developed as a medicine for insomnia.

### BRIEF SUMMARY OF THE INVENTION

It is an object of the present invention to provide a cyclopropane derivative which has orexin receptor antagonism and is useful as an agent for treating sleep disorder or dyssomnia caused by an orexin receptor, including insomnia as a typical example, and a medicinal use thereof.

The present invention relates to the following [1] to [17]:

[1] A compound represented by the following formula (I) or a pharmaceutically acceptable salt thereof:

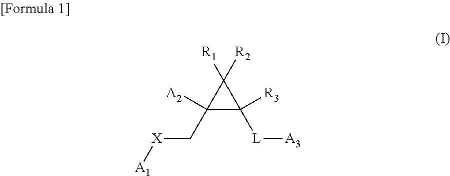

wherein

A₁ represents an aryl group selected from Group 1, which may optionally have 1 to 3 substituents selected from Substituent group α or a heteroaryl group selected from Group 2, which may optionally have 1 to 3 substituents selected from Substituent group α,

A₂ and A₃ each independently represent an aryl group selected from Group 1, which may optionally have 1 to 3 substituents selected from Substituent group α or a heterocyclyl group selected from Group 3, which may optionally have 1 to 3 substituents selected from Substituent group α,

R₁, R₂ and R₃ each independently represent a hydrogen atom, a halogen atom or a C₁₋₆ alkyl group which may optionally have 1 to 3 substituents selected from Substituent group β,

X represents an oxygen atom, a C₁₋₆ alkylene group, a formula —NR₄— (wherein R₄ represents a hydrogen atom or a C₁₋₆ alkyl group) or a sulfonyl group,

L represents a bond or a formula —CONR₅— (wherein R₅ represents a hydrogen atom or a C₁₋₆ alkyl group), wherein

Group 1: a phenyl group, a naphthyl group, an azulenyl group, an anthryl group and a phenanthryl group;

Group 2: a furyl group, a thienyl group, a pyrrolyl group, an imidazolyl group, a triazolyl group, a tetrazolyl group, a thiazolyl group, a pyrazolyl group, an oxazolyl group, an isoxazolyl group, an isothiazolyl group, a furazanyl group, a thiadiazolyl group, an oxadiazolyl group, a pyridyl group, a pyrazinyl group, a pyridazinyl group, a triazinyl group, an indolyl group, an isoindolyl group, an indazolyl group, a benzoxazolyl group, a benzisoxadiazolyl group, a benzothiazolyl group, a benzisothiazolyl group, a quinolyl group and an isoquinolyl group;

Group 3: a furyl group, a thienyl group, a pyrrolyl group, an imidazolyl group, a triazolyl group, a tetrazolyl group, a thiazolyl group, a pyrazolyl group, an oxazolyl group, an isoxazolyl group, an isothiazolyl group, a furazanyl group, a thiadiazolyl group, an oxadiazolyl group, a pyridyl group, a pyrazinyl group, a pyridazinyl group, a pyrimidinyl group, a triazinyl group, a 2-pyridonyl group, a 4-pyridonyl group, a pyridazidonyl group, a pyrimididonyl group, a purinyl group, a pteridinyl group, a quinolyl group, an isoquinolyl group, a naphthylidyl group, a quinoxalyl group, a cinnolyl group, a quinazolyl group, a phthalazyl group, an imidazopyridyl group, an imidazothiazolyl group, an imidazoxazolyl group, a benzimidazolyl group, an indolyl group, an isoindolyl group, an indazolyl group, a pyrrolopyridyl group, a thienopyridyl group, a fluoropyridyl group, a benzoxazolyl group, a benzisoxadiazolyl group, a benzothiazolyl group, a benzisothiazolyl group, a pyridopyrimidinyl group, a benzofuryl group, a benzothienyl group, a benzothiadiazolyl group, a benzo[1,3]dioxolyl group, a thienofuryl group, a dihydroisobenzofuranyl group, a chromanyl group, an isochromanyl group, a 1,3-dioxaindanyl group, a 1,4-dioxatetralinyl group and dihydrobenzo[1,4]oxazinyl group;

Substituent group α: a cyano group, a halogen atom, a formula —NR₆R₇ (wherein R₆ and R₇ each independently represent a hydrogen atom or a C₁₋₆ alkyl group), a C₁₋₆ alkyl group which may optionally have 1 to 3 substituents selected from Substituent group β, a C₁₋₆ alkoxy group which may optionally have 1 to 3 substituents selected from Substituent group β, a C₁₋₆ alkylcarbonyl group which may optionally have 1 to 3 substituents selected from Substituent group β, a C₁₋₆ alkylsulfonyl group which may optionally have 1 to 3 substituents selected from Substituent group β, an aryl group selected from group 1, which may optionally have 1 to 3 substituents selected from Substituent group β, and a heterocyclyl group selected from group 3, which may optionally have 1 to 3 substituents selected from Substituent group β; and

Substituent group β: a cyano group, a halogen atom, a hydroxy group, a C₃₋₈ cycloalkyl group and a C₁₋₆ alkoxy group.

[2] The compound according to [1] above, which is represented by the following formula (II), or a pharmaceutically acceptable salt thereof:

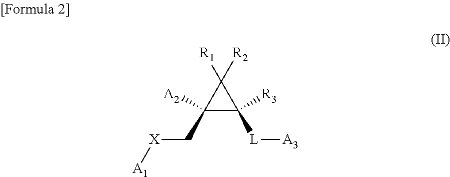

wherein A₁, A₂, A₃, R₁, R₂, R₃, X and L have the same definitions as those described in [1] above.
[3] The compound according to [1] or [2] above, or a pharmaceutically acceptable salt thereof, wherein R₁, R₂ and R₃ each represent a hydrogen atom.
[4] The compound according to [3] above, or a pharmaceutically acceptable salt thereof, wherein L represents a formula —CONH—.

[5] The compound according to [4] above, or a pharmaceutically acceptable salt thereof, wherein X represents an oxygen atom.

[6] The compound according to [4] above, or a pharmaceutically acceptable salt thereof, wherein X represents methylene.
[7] The compound according to [5] or [6] above, or a pharmaceutically acceptable salt thereof, wherein A₂ and A₃ each independently represent an aryl group or a heterocyclyl group which may optionally have 1 to 3 substituents selected from a cyano group, a halogen atom, a C₁₋₆ alkyl group, a halo-C₁₋₆ alkyl group and a C₁₋₆ alkoxy group.
[8] The compound according to [7] above, or a pharmaceutically acceptable salt thereof, wherein A₂ and A₃ each independently represent a phenyl group, a naphthyl group, a furyl group, a thienyl group, a pyrrolyl group, an imidazolyl group, a triazolyl group, a tetrazolyl group, a thiazolyl group, a pyrazolyl group, an oxazolyl group, an isoxazolyl group, an isothiazolyl group, a furazanyl group, a thiadiazolyl group, an oxadiazolyl group, a pyridyl group, a pyrazinyl group, a pyridazinyl group, a pyrimidinyl group, a triazinyl group, a quinolyl group or an isoquinolyl group, which may optionally have 1 to 3 substituents selected from a cyano group, a halogen atom, a C₁₋₆ alkyl group, a halo-C₁₋₆ alkyl group and C₁₋₆ alkoxy group.
[9] The compound according to [8] above, or a pharmaceutically acceptable salt thereof, wherein A₂ represents a phenyl group which may optionally have 1 to 3 substituents selected from a cyano group, a halogen atom, a C₁₋₆ alkyl group, a halo-C₁₋₆ alkyl group and a C₁₋₆ alkoxy group.
[10] The compound according to [9] above, or a pharmaceutically acceptable salt thereof, wherein A₃ represents a phenyl group or a pyridyl group which may optionally have 1 to 3 substituents selected from a cyano group, a halogen atom, a C₁₋₆ alkyl group, a halo-C₁₋₆ alkyl group and a C₁₋₆ alkoxy group.
[11] The compound according to [10] above, or a pharmaceutically acceptable salt thereof, wherein A₁ represents a phenyl group, a pyrazolyl group or a triazolyl group which may optionally have 1 to 3 substituents selected from a halogen atom, a C₁₋₆ alkyl group, a halo-C₁₋₆ alkyl group, a C₁₋₆ alkoxy group, a C₃₋₈ cycloalkyl group and a C₁₋₆ alkoxy group.
[12] The compound, which is selected from the following compounds:
1) N-phenyl-2-[(3,4-dimethoxyphenyl)oxymethyl]-2-phenylcyclopropanecarboxamide,
2) N-methyl-N-phenyl-2-[(3,4-dimethoxyphenyl)oxymethyl]-2-phenylcyclopropanecarboxamide,
3) N-(5-fluoropyridin-2-yl)-2-[(3,4-dimethoxyphenyl)oxymethyl]-2-phenylcyclopropanecarboxamide,
4) (1R,2S)—N-(5-cyanopyridin-2-yl)-2-[(3,4-dimethoxyphenyl)oxymethyl]-2-phenylcyclopropanecarboxamide,
5) N-(4-methyl-1,3-thiazol-2-yl)-2-[(3,4-dimethoxyphenyl)oxymethyl]-2-phenylcyclopropanecarboxamide,
6) N-(5-chloropyridin-2-yl)-2-[(3,4-dimethoxyphenyl)oxymethyl]-2-phenylcyclopropanecarboxamide,
7) N-(3-methyl-1,2,4-thiadiazol-5-yl)-2-[(3,4-dimethoxyphenyl)oxymethyl]-2-phenylcyclopropanecarboxamide,
8) N-(3-methylisoxazol-5-yl)-2-[(3,4-dimethoxyphenyl)oxymethyl]-2-phenylcyclopropanecarboxamide,
9) N-(1,5-dimethyl-1H-pyrazol-3-yl)-2-[(3,4-dimethoxyphenyl)oxymethyl]-2-phenylcyclopropanecarboxamide,
10) N-(5-fluoropyridin-2-yl)-2-[(3,4-dimethoxyphenyl)oxymethyl]-2-phenylcyclopropanecarboxamide,
11) N-(5-chloropyridin-3-yl)-2-[(3,4-dimethoxyphenyl)oxymethyl]-2-phenylcyclopropanecarboxamide,
12) N-(2-methylpyridin-4-yl)-2-[(3,4-dimethoxyphenyl)oxymethyl]-2-phenylcyclopropanecarboxamide,
13) N-(5-chloro-1,3-thiazol-2-yl)-2-[(3,4-dimethoxyphenyl)oxymethyl]-2-phenylcyclopropanecarboxamide,
14) N-(3-chloropyridin-4-yl)-2-[(3,4-dimethoxyphenyl)oxymethyl]-2-phenylcyclopropanecarboxamide,
15) N-(5-methylpyridin-3-yl)-2-[(3,4-dimethoxyphenyl)oxymethyl]-2-phenylcyclopropanecarboxamide,
16) N-(1,3,4-thiadiazol-2-yl)-2-[(3,4-dimethoxyphenyl)oxymethyl]-2-phenylcyclopropanecarboxamide,
17) N-(5-methyl-1,3,4-thiadiazol-2-yl)-2-[(3,4-dimethoxyphenyl)oxymethyl]-2-phenylcyclopropanecarboxamide,
18) N-(4-cyanopyridin-2-yl)-2-[(3,4-dimethoxyphenyl)oxymethyl]-2-phenylcyclopropanecarboxamide,
19) N-(2-fluoropyridin-4-yl)-2-[(3,4-dimethoxyphenyl)oxymethyl]-2-phenylcyclopropanecarboxamide,
20) N-(3-methylpyridin-4-yl)-2-[(3,4-dimethoxyphenyl)oxymethyl]-2-phenylcyclopropanecarboxamide,
21) N-(5-methylisoxazol-3-yl)-2-[(3,4-dimethoxyphenyl)oxymethyl]-2-phenylcyclopropanecarboxamide,
22) N-(5-methyl-1,3-thiazol-2-yl)-2-[(3,4-dimethoxyphenyl)oxymethyl]-2-phenylcyclopropanecarboxamide,
23) N-(4-methyl-1,3-thiazol-2-yl)-2-[(3,4-dimethoxyphenyl)oxymethyl]-2-phenylcyclopropanecarboxamide,
24) N-(5-fluoropyridin-3-yl)-2-[(3,4-dimethoxyphenyl)oxymethyl]-2-phenylcyclopropanecarboxamide,
25) N-(3-trifluoromethylpyridin-4-yl)-2-[(3,4-dimethoxyphenyl)oxymethyl]-2-phenylcyclopropanecarboxamide,
26) N-(6-fluoromethylpyridin-3-yl)-2-[(3,4-dimethoxyphenyl)oxymethyl]-2-phenylcyclopropanecarboxamide,
27) N-(6-fluoropyridin-2-yl)-2-[3,4-dimethoxyphenyl)oxymethyl]-2-phenylcyclopropanecarboxamide,
28) N-(4-chloropyridin-2-yl)-2-[(3,4-dimethoxyphenyl)oxymethyl]-2-phenylcyclopropanecarboxamide,
29) N-(5-cyanopyridin-2-yl)-2-[(3,4-dimethoxyphenyl)oxymethyl]-2-phenylcyclopropanecarboxamide,
30) N-(6-chloropyridazin-3-yl)-2-[(3,4-dimethoxyphenyl)oxymethyl]-2-phenylcyclopropanecarboxamide,
31) N-(6-cyanopyridin-3-yl)-2-[(3,4-dimethoxyphenyl)oxymethyl]-2-phenylcyclopropanecarboxamide,
32) N-(2-chloropyridin-4-yl)-2-[(3,4-dimethoxyphenyl)oxymethyl]-2-phenylcyclopropanecarboxamide,
33) N-(pyrimidin-4-yl)-2-[(3,4-dimethoxyphenyl)oxymethyl]-2-phenylcyclopropanecarboxamide,
34) N-(3-methoxyphenyl)-2-[(3,4-dimethoxyphenyl)oxymethyl]-2-phenylcyclopropanecarboxamide,
35) N-[2-(1H-1,2,4-triazol-3-yl)phenyl]-2-[(3,4-dimethoxyphenyl)oxymethyl]-2-phenylcyclopropanecarboxamide,
36) N-(4-methylpyridin-2-yl)-2-[(3,4-dimethoxyphenyl)oxymethyl]-2-phenylcyclopropanecarboxamide,
37) N-(6-methylpyridin-2-yl)-2-[(3,4-dimethoxyphenyl)oxymethyl]-2-phenylcyclopropanecarboxamide,
38) N-(6-chloropyridin-2-yl)-2-[(3,4-dimethoxyphenyl)oxymethyl]-2-phenylcyclopropanecarboxamide,
39) N-(1-ethyl-1H-pyrazol-5-yl)-2-[(3,4-dimethoxyphenyl)oxymethyl]-2-phenylcyclopropanecarboxamide,
40) N-(3-methylisothiazol-5-yl)-2-[(3,4-dimethoxyphenyl)oxymethyl]-2-phenylcyclopropanecarboxamide,
41) (1R,2S)—N-(pyridin-2-yl)-2-[(3,4-dimethoxyphenyl)oxymethyl]-2-phenylcyclopropanecarboxamide,
42) N-(pyridin-3-yl)-2-[(3,4-dimethoxyphenyl)oxymethyl]-2-phenylcyclopropanecarboxamide,
43) N-(pyridin-4-yl)-2-[(3,4-dimethoxyphenyl)oxymethyl]-2-phenylcyclopropanecarboxamide,
44) 2-{[(3,4-dimethoxyphenoxy)methyl]-2-phenylcyclopropyl}-5-fluoro-1H-benzimidazole,
45) 3-{2-[(3,4-dimethoxyphenoxy)methyl]-2-phenylcyclopropyl}-5-phenyl-1H-1,2,4-triazole,
46) N-(pyridin-2-yl)-2-(3-methoxyphenyloxymethyl)-2-phenylcyclopropanecarboxamide,
47) N-(pyridin-2-yl)-2-(4-methoxyphenyloxymethyl)-2-phenylcyclopropanecarboxamide,
48) N-(pyridin-2-yl)-2-[(3-methoxy-4-methoxymethylphenyl)oxymethyl]-2-phenylcyclopropanecarboxamide,
49) N-(pyridin-2-yl)-2-(3,4-dimethoxyphenyl)-2-phenyloxymethylcyclopropanecarboxamide,
50) N-(pyridin-2-yl)-2-[(4-methoxy-3-methoxymethylphenyl)oxymethyl]-2-phenylcyclopropanecarboxamide,
51) N-(pyridin-2-yl)-2-(3,4-dimethoxyphenyl)-2-(4-fluorophenyloxymethyl)cyclopropanecarboxamide,
52) N-(5-fluoropyridin-2-yl)-2-(3,4-dimethoxyphenyl)-2-(4-fluorophenyloxymethyl)cyclopropanecarboxamide,
53) N-(5-chloropyridin-2-yl)-2-[(1-methyl-6-oxo-1,6-dihydropyridin-3-yl)oxymethyl]-2-phenylcyclopropanecarboxamide,
54) N-(5-chloropyridin-2-yl)-2-[(1-ethyl-6-oxo-1,6-dihydropyridin-3-yl)oxymethyl]-2-phenylcyclopropanecarboxamide,
55) N-(5-fluoropyridin-2-yl)-2-[(1-ethyl-6-oxo-1,6-dihydropyridin-3-yl)oxymethyl]-2-phenylcyclopropanecarboxamide,
56) N-(pyridin-2-yl)-2-[(3,4-dimethoxyphenyl)oxymethyl]-2-(pyridin-2-yl)cyclopropanecarboxamide,
57) (1S,2R)—N-(5-cyanopyridin-2-yl)-2-[(1-ethyl-3-methyl-1H-pyrazol-4-yl)oxymethyl]-2-phenylcyclopropanecarboxamide,
58) (1S,2R)—N-(pyridin-2-yl)-2-[(1-ethyl-3-methyl-1H-pyrazol-4-yl)oxymethyl]-2-phenylcyclopropanecarboxamide,
59) (1S,2R)—N-(6-fluoropyridin-3-yl)-2-[(1-ethyl-3-methyl-1H-pyrazol-4-yl)oxymethyl]-2-phenylcyclopropanecarboxamide,
60) (1S,2R)—N-(4-fluorophenyl)-2-[(1-ethyl-3-methyl-1H-pyrazol-4-yl)oxymethyl]-2-phenylcyclopropanecarboxamide,
61) (1S,2R)—N-(5-methoxypyridin-3-yl)-2-[(1-ethyl-3-methyl-1H-pyrazol-4-yl)oxymethyl]-2-phenylcyclopropanecarboxamide,
62) (1S,2R)—N-(2-fluoropyridin-4-yl)-2-[(1-ethyl-3-methyl-1H-pyrazol-4-yl)oxymethyl]-2-phenylcyclopropanecarboxamide,
63) N-(5-cyanopyridin-2-yl)-2-[(1-ethyl-1H-pyrazol-4-yl)oxymethyl]-2-phenylcyclopropanecarboxamide,
64) (1R,2S)-2-[(1-ethyl-3-methyl-1H-pyrazol-4-yl)oxymethyl]-2-(3-fluorophenyl)-N-(pyridin-2-yl)cyclopropanecarboxamide,
65) (1R,2S)-2-[(1-ethyl-3-methyl-1H-pyrazol-4-yl)oxymethyl]-2-(3-fluorophenyl)-N-(5-fluoropyridin-2-yl)cyclopropanecarboxamide,
66) (1R,2S)-2-[(1-ethyl-3-methyl-1H-pyrazol-4-yl)oxymethyl]-2-(3-fluorophenyl)-N-(5-fluoropyridin-3-yl)cyclopropanecarboxamide,
67) (1R,2S)-2-[(1-ethyl-3-methyl-1H-pyrazol-4-yl)oxymethyl]-2-(3-fluorophenyl)-N-(4-fluorophenyl)cyclopropanecarboxamide,
68) (1R,2S)-2-[(1-ethyl-3-methyl-1H-pyrazol-4-yl)oxymethyl]-N-(5-fluoro-4-methoxypyridin-2-yl)-2-(3-fluorophenyl)cyclopropanecarboxamide,
69) (1R,2S)—N-(5-cyanopyridin-2-yl)-2-[(1-ethyl-3-methyl-1H-pyrazol-4-yl)oxymethyl]-2-(3-fluorophenyl)cyclopropanecarboxamide,
70) (1R,2S)-2-[(1-ethyl-3-methyl-1H-pyrazol-4-yl)oxymethyl]-2-(3-fluorophenyl)-N-(2-fluoropyridin-4-yl)cyclopropanecarboxamide,
71) (1R,2S)-2-[(1,3-dimethyl-6-oxo-1,6-dihydropyridazin-4-yl)oxymethyl]-N-(5-methoxypyridin-3-yl)-2-phenylcyclopropanecarboxamide,
72) (1R,2S)-2-[(1,3-dimethyl-6-oxo-1,6-dihydropyridazin-4-yl)oxymethyl]-N-(4-fluorophenyl)-2-phenylcyclopropanecarboxamide,
73) (1R,2S)-2-[(1,3-dimethyl-6-oxo-1,6-dihydropyridazin-4-yl)oxymethyl]-N-(5-fluoro-4-methylpyridin-3-yl)-2-phenylcyclopropanecarboxamide,
74) (1R,2S)—N-(5-chloropyridin-3-yl)-2-[(1,3-dimethyl-6-oxo-1,6-dihydropyridazin-4-yl)oxymethyl]-2-phenylcyclopropanecarboxamide,
75) (1R,2S)-2-[2-(3-difluoromethyl-5-methyl-1H-1,2,4-triazol-1-yl)ethyl]-N-(5-fluoro-4-methylpyridin-2-yl)-2-phenylcyclopropanecarboxamide,
76) (1R,2S)-2-[2-(3-difluoromethyl-5-methyl-1H-1,2,4-triazol-1-yl)ethyl]-N-(5-fluoropyridin-2-yl)-2-phenylcyclopropanecarboxamide,
77) (1R,2S)—N-(5-chloropyridin-2-yl)-2-[2-(3-difluoromethyl-5-methyl-1H-1,2,4-triazol-1-yl)ethyl]-2-phenylcyclopropanecarboxamide,
78) (1R,2S)-2-[2-(3-difluoromethyl-5-methyl-1H-1,2,4-triazol-1-yl)ethyl]-N-(4-fluorophenyl)-2-phenylcyclopropanecarboxamide,
79) (1R,2S)—N-(5-fluoropyridin-2-yl)-2-[2-(3-difluoromethyl-5-methyl-1H-1,2,4-triazol-1-yl)ethyl]-2-phenylcyclopropanecarboxamide,
80) N-(5-fluoropyridin-2-yl)-2-[2-(3-methoxy-4-oxopyridine-1(4H)-yl)ethyl]-2-phenyl cyclopropanecarboxamide,
81) 2-[2-(3,5-dimethyl-1H-1,2,4-triazol-1-yl)ethyl]-N-(5-fluoropyridin-2-yl)-2-phenylcyclopropanecarboxamide,
82) N-(5-chloropyridin-2-yl)-2-[2-(3,5-dimethyl-1H-1,2,4-triazol-1-yl)ethyl]-2-phenylcyclopropanecarboxamide,
83) (1R,2S)—N-(5-fluoropyridin-2-yl)-2-[2-(5-methyl-3-trifluoromethyl-1H-1,2,4-triazol-1-yl)ethyl]-2-phenylcyclopropanecarboxamide,
84) (1R,2S)—N-(5-chloropyridin-2-yl)-2-[2-(5-methyl-3-trifluoromethyl-1H-1,2,4-triazol-1-yl)ethyl]-2-phenylcyclopropanecarboxamide,
85) (1R,2S)—N-(5-cyanopyridin-2-yl)-2-[2-(5-methyl-3-trifluoromethyl-1H-1,2,4-triazol-1-yl)ethyl]-2-phenylcyclopropanecarboxamide,
86) (1R,2S)—N-(5-fluoropyridin-2-yl)-2-[2-(3-cyclopropyl-5-methyl-1H-1,2,4-triazol-1-yl)ethyl]-2-phenylcyclopropanecarboxamide,
87) (1R,2S)—N-(5-chloropyridin-2-yl)-2-[2-(3-cyclopropyl-5-methyl-1H-1,2,4-triazol-1-yl)ethyl]-2-phenylcyclopropanecarboxamide,
88) (1R,2S)—N-(5-cyanopyridin-2-yl)-2-[2-(3-cyclopropyl-5-methyl-1H-1,2,4-triazol-1-yl)ethyl]-2-phenylcyclopropanecarboxamide,
89) (1R,2S)—N-(5-fluoropyridin-2-yl)-2-[2-(3-ethyl-5-methyl-1H-1,2,4-triazol-1-yl)ethyl]-2-phenylcyclopropanecarboxamide, and
90) (1R,2S)—N-(5-chloropyridin-2-yl)-2-[2-(3-ethyl-5-methyl-1H-1,2,4-triazol-1-yl)ethyl]-2-phenylcyclopropanecarboxamide,
   or a pharmaceutically acceptable salt thereof
   [13] A pharmaceutical composition comprising, as an active ingredient, the compound according to any one of [1] to [12] above or a pharmaceutically acceptable salt thereof
   [14] The pharmaceutical composition according to [13] above, for the treatment of sleep disorder for which orexin receptor antagonism is effective.
   [15] The pharmaceutical composition according to [14] above, wherein said sleep disorder is insomnia.
   [16] A method for treating sleep disorder for which orexin receptor antagonism is effective, which comprises administering the compound according to any one of [1] to [12] above or a pharmaceutically acceptable salt thereof into a subject in need thereof
   [17] The method according to [16] above, wherein said sleep disorder is insomnia.

The cyclopropane derivative according to the present invention or a pharmaceutically acceptable salt thereof has orexin receptor antagonism. Therefore, the cyclopropane compound or a pharmaceutically acceptable salt thereof has a potential of usefulness for the treatment of sleep disorder for which orexin receptor antagonism is effective, for example, insomnia.

### DETAILED DESCRIPTION OF THE INVENTION

Hereinafter, the meanings of symbols, terms and the like used in the specification of the present application will be explained, and thus, the present invention will be described in detail.

In the specification of the present application, the structural formula of a compound may indicate a certain isomer for convenience sake. The present invention includes all isomers generated due to the structure of the compound, such as geometric isomers, optical isomers based on asymmetric carbon atoms, steric isomers or tautomers, and the isomeric mixtures thereof. Thus, the compound of the present invention is not limited to the descriptions of a formula given for convenience, and it may be either an isomer or a mixture. Accordingly, there may be a case in which the compound has asymmetric carbon atoms in a molecule thereof and an optically active form and a racemic form exist. However, the present invention is not limited thereto, but it includes all cases. Moreover, there may also be a case in which crystal polymorphisms exist. The present invention is not limited thereto, either, and it includes single crystals or the mixtures thereof. Other than anhydrides, hydrates may also be included. Solvates may be used. These substances are all included in the scope of claims in the specification of the present application.

The present invention includes a compound formed by isotopically labeling the compound of the formula (I). This compound is identical to the compound of the formula (I) with the exception that one or more atoms thereof are substituted with atom(s) having an atomic mass or mass number that are different from those generally found in the nature. Examples of an isotope that can be included in the compound of the present invention include the isotopes of hydrogen, carbon, nitrogen, oxygen, phosphorus, fluorine, iodine and chloride. Specific examples include ²H, ³H, ¹¹C, ¹⁴C, ¹³N, ¹⁵O, ¹⁸F, ³⁵S, ¹²³I and ¹²⁵I.

The compound of the present invention and a pharmaceutically acceptable derivative thereof (e.g. a salt), which include the above described isotopes and/or other isotopes, are included in the scope of claims set forth in the specification of the present application. The isotopically labeled compound of the present invention, for example, a compound, into which a radioisotope(s) such as ³H and/or ¹⁴C are included, is useful for the tissue distribution assay of a pharmaceutical agent and/or a substrate. Isotopes ³H and ¹⁴C are considered useful because of the easiness of preparation and detection. Isotopes ¹¹C and ¹⁸F are considered useful for PET (positron-emission tomography), and isotope ¹²⁵I is considered useful for SPECT (single-photon-emission computed tomography) and these isotopes are useful for brain imaging. Substitution with a heavy isotope such as ²H is advantageous for a certain type of therapy, such as an increase in the in vivo half-life or a decrease in necessary dose due to its higher metabolic stability. Thus, such a heavy isotope is considered useful under certain circumstances. The isotopically labeled compound of the formula (I) of the present invention can be uniformly prepared by performing procedures disclosed in schemes and/or Examples as described below, using commonly used isotopically labeled reagents, instead of non-isotopically labeled reagents.

In the present specification, the term “halogen atom” is used to mean a fluorine atom, a chlorine atom, a bromine atom, an iodine atom, etc. It is preferably a fluorine atom or a chloride atom.

The term “C₁₋₆ alkyl group” is used to mean an alkyl group containing 1 to 6 carbon atoms. Examples of a preferred C₁₋₆ alkyl group include linear or branched alkyl groups such as a methyl group, an ethyl group, an n-propyl group, an isopropyl group, an n-butyl group, an isobutyl group, a t-butyl group, an n-pentyl group, an isopentyl group, a neopentyl group, an n-hexyl group, a 1-methylpropyl group, a 1,2-dimethylpropyl group, a 1-ethylpropyl group, a 1-methyl-2-ethylpropyl group, a 1-ethyl-2-methylpropyl group, a 1,1,2-trimethylpropyl group, a 1-methylbutyl group, a 2-methylbutyl group, a 1,1-dimethylbutyl group, a 2,2-dimethylbutyl group, a 2-ethylbutyl group, a 1,3-dimethylbutyl group, a 2-methylpentyl group and a 3-methylpentyl group. Of these, a methyl group, an ethyl group and an n-propyl group are more preferable.

The term “C₁₋₆ alkylene group” is used to mean an alkylene group containing 1 to 6 carbon atoms. Examples of a preferred C₁₋₆ alkylene group include linear or branched alkylene groups such as a methylene group, an ethylene group, an n-propylene group, an isopropylene group, an n-butylene group, an isobutylene group, an n-pentylene group, an isopentylene group and a neopentylene group. Of these, a methylene group, an ethylene group and an n-propylene group are more preferable.

The term “C₁₋₆ alkoxy group” is used to mean an alkyl group containing 1 to 6 carbon atoms, in which one hydrogen atom is substituted with an oxygen atom. Examples of such a C₁₋₆ alkoxy group include a methoxy group, an ethoxy group, an n-propoxy group, an isopropoxy group, an n-butoxy group, an isobutoxy group, a sec-butoxy group, a t-butoxy group, an n-pentoxy group, an isopentoxy group, a sec-pentoxy group, a t-pentoxy group, an n-hexoxy group, an isohexoxy group, a 1,2-dimethylpropoxy group, a 2-ethylpropoxy group, a 1-methyl-2-ethylpropoxy group, a 1-ethyl-2-methylpropoxy group, a 1,1,2-trimethylpropoxy group, a 1,1-dimethylbutoxy group, a 2,2-dimethylbutoxy group, a 2-ethylbutoxy group, a 1,3-dimethylbutoxy group, a 2-methylpentoxy group, a 3-methylpentoxy group and a hexyloxy group.

The term “C₁₋₆ alkylcarbonyl group” is used to mean an alkyl group containing 1 to 6 carbon atoms, in which one hydrogen atom is substituted with a carbonyl group. Examples of a preferred C₁₋₆ alkylcarbonyl group include an acetyl group, a propionyl group and a butyryl group.

The term “C₁₋₆ alkylsulfonyl group” is used to mean an alkyl group containing 1 to 6 carbon atoms, in which one hydrogen atom is substituted with a sulfonyl group. Examples of such a C₁₋₆ alkylsulfonyl group include a methyl sulfonyl group, an ethylsulfonyl group, an n-propylsulfonyl group, an isopropylsulfonyl group, an n-butylsulfonyl group, an isobutylsulfonyl group, a t-butylsulfonyl group, an n-pentylsulfonyl group, an isopentylsulfonyl group, a neopentylsulfonyl group, an n-hexylsulfonyl group and a 1-methylpropylsulfonyl group.

The term “C₃₋₈ cycloalkyl group” is used to mean a cyclic alkyl group containing 3 to 8 carbon atoms. Examples of a preferred C₃₋₈ cycloalkyl group include a cyclopropyl group, a cyclobutyl group, a cyclopentyl group, a cyclohexyl group, a cycloheptyl group and a cyclooctyl group.

The term “aryl group” is used to mean an aryl group selected from Group 1. Group 1 consists of a phenyl group, a naphthyl group, an azulenyl group, an anthryl group and a phenanthryl group.

The term “heteroaryl group” is used to mean a heteroaryl group selected from Group 2. Group 2 consists of a furyl group, a thienyl group, a pyrrolyl group, an imidazolyl group, a triazolyl group, a tetrazolyl group, a thiazolyl group, a pyrazolyl group, an oxazolyl group, an isoxazolyl group, an isothiazolyl group, a furazanyl group, a thiadiazolyl group, an oxadiazolyl group, a pyridyl group, a pyrazinyl group, a pyridazinyl group, a triazinyl group, an indolyl group, an isoindolyl group, an indazolyl group, a benzoxazolyl group, a benzisoxadiazolyl group, a benzothiazolyl group, a benzisothiazolyl group, a quinolyl group and an isoquinolyl group.

The term “heterocyclyl group” is used to mean an aryl group selected from Group 3. Group 3 consists of a furyl group, a thienyl group, a pyrrolyl group, an imidazolyl group, a triazolyl group, a tetrazolyl group, a thiazolyl group, a pyrazolyl group, an oxazolyl group, an isoxazolyl group, an isothiazolyl group, a furazanyl group, a thiadiazolyl group, an oxadiazolyl group, a pyridyl group, a pyrazinyl group, a pyridazinyl group, a pyrimidinyl group, a triazinyl group, a 2-pyridonyl group, a 4-pyridonyl group, a pyridazidonyl group, a pyrimididonyl group, a purinyl group, a pteridinyl group, a quinolyl group, an isoquinolyl group, a naphthylidyl group, a quinoxalyl group, a cinnolyl group, a quinazolyl group, a phthalazyl group, an imidazopyridyl group, an imidazothiazolyl group, an imidazoxazolyl group, a benzimidazolyl group, an indolyl group, an isoindolyl group, an indazolyl group, a pyrrolopyridyl group, a thienopyridyl group, a fluoropyridyl group, a benzoxazolyl group, a benzisoxadiazolyl group, a benzothiazolyl group, a benzisothiazolyl group, a pyridopyrimidinyl group, an oxodihydropyridopyrimidinyl group, a benzofuryl group, a benzothienyl group, a benzothiadiazolyl group, a benzo[1,3]dioxolyl group, a thienofuryl group, a dihydroisobenzofuranyl group, a chromanyl group, an isochromanyl group, a 1,3-dioxaindanyl group, a 1,4-dioxatetralinyl group and a dihydrobenzo[1,4]oxazinyl group.

The term “Substituent group α” is used to mean a group consisting of a cyano group, a halogen atom, a formula —NR₆R₇ (wherein R₆ and R₇ each independently represent a hydrogen atom or a C₁₋₆ alkyl group), a C₁₋₆ alkyl group which may optionally have 1 to 3 substituents selected from Substituent group β, a C₁₋₆ alkoxy group which may optionally have 1 to 3 substituents selected from Substituent group β, a C₁₋₆ alkylcarbonyl group which may optionally have 1 to 3 substituents selected from Substituent group β, a C₁₋₆ alkylsulfonyl group which may optionally have 1 to 3 substituents selected from Substituent group β, an aryl group selected from group 1, which may optionally have 1 to 3 substituents selected from Substituent group β, and a heterocyclyl group selected from group 3, which may optionally have 1 to 3 substituents selected from Substituent group β.

The term “Substituent group β” is used to mean a group consisting of a cyano group, a halogen atom, a hydroxyl group, a C₃₋₈ cycloalkyl group and a C₁₋₆ alkoxy group.

The cyclopropane derivative of the formula (I) of the present invention may also be a pharmaceutically acceptable salt. Specific examples of such a pharmaceutically acceptable salt include inorganic acid salts (for example, a sulfate, a nitrate, a perchlorate, a phosphate, a carbonate, a bicarbonate, a hydrofluoride, a hydrochloride, a hydrobromide, a hydroiodide); organic carboxylates (for example, an acetate, an oxalate, a maleate, a tartrate, a fumarate, a citrate); organic sulfonates (for example, a methanesulfonate, a trifluoromethanesulfonate, an ethanesulfonate, a benzenesulfonate, a toluenesulfonate, a camphorsulfonate); amino acid salts (for example, an aspartate, a glutamate); quaternary amine salts; alkaline metal salts (for example, a sodium salt, a potassium salt); and alkaline-earth metal salts (for example, a magnesium salt, a calcium salt).

The cyclopropane derivative of the formula (I) of the present invention or a pharmaceutically acceptable salt thereof is preferably a cyclopropane derivative represented by the following formula (II) or a pharmaceutically acceptable salt thereof:

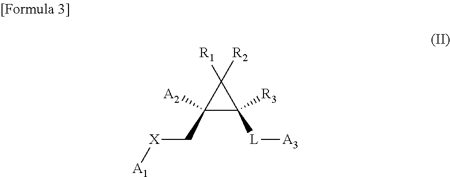

wherein A₁, A₂, A₃, R₁, R₂, R₃, X and L have the same definitions as those described above.

Furthermore, the cyclopropane derivative of the present invention or a pharmaceutically acceptable salt thereof is preferably a cyclopropane derivative or a pharmaceutically acceptable salt thereof, wherein in the formula (I) or the formula (II), R₁, R₂ and R₃ each represent a hydrogen atom.

Moreover, a cyclopropane derivative or a pharmaceutically acceptable salt thereof, wherein in the formula (I) or the formula (II), L represents a formula —CONH—, is preferable; and a cyclopropane derivative or a pharmaceutically acceptable salt thereof, wherein in the formula (I) or the formula (II), X represents an oxygen atom or a methylene group, is particularly preferable.

The cyclopropane derivative of the present invention or a pharmaceutically acceptable salt thereof is preferably a cyclopropane derivative or a pharmaceutically acceptable salt thereof, wherein in the formula (I) or the formula (II), A₂ and A₃ each independently represent an aryl group or a heterocyclyl group, which may optionally have 1 to 3 substituents selected from a cyano group, a halogen atom, a C₁₋₆ alkyl group, a halo-C₁₋₆ alkyl group and a C₁₋₆ alkoxy group.

Furthermore, a cyclopropane derivative or a pharmaceutically acceptable salt thereof, wherein A₂ and A₃ each independently represent an aryl group or a heteroaryl group, which may optionally have 1 to 3 substituents selected from a cyano group, a halogen atom, a C₁₋₆ alkyl group, a halo-C₁₋₆ alkyl group and a C₁₋₆ alkoxy group, is preferable; and a cyclopropane derivative or a pharmaceutically acceptable salt thereof, wherein A₂ and A₃ each independently represent a phenyl group, a naphthyl group, a furyl group, a thienyl group, a pyrrolyl group, an imidazolyl group, a triazolyl group, a tetrazolyl group, a thiazolyl group, a pyrazolyl group, an oxazolyl group, an isoxazolyl group, an isothiazolyl group, a furazanyl group, a thiadiazolyl group, an oxadiazolyl group, a pyridyl group, a pyrazinyl group, a pyridazinyl group, a pyrimidinyl group, a triazinyl group, a quinolyl group or an isoquinolyl group, which may optionally have 1 to 3 substituents selected from a cyano group, a halogen atom, a C₁₋₆ alkyl group, a halo-C₁₋₆ alkyl group and a C₁₋₆ alkoxy group is particularly preferable.

Among others, a cyclopropane derivative or a pharmaceutically acceptable salt thereof, wherein A₂ represents a phenyl group which may optionally have 1 to 3 substituents selected from a cyano group, a halogen atom, a C₁₋₆ alkyl group, a halo-C₁₋₆ alkyl group and a C₁₋₆ alkoxy group; a cyclopropane derivative or a pharmaceutically acceptable salt thereof, wherein A₃ represents a phenyl group or a pyridyl group which may optionally have 1 to 3 substituents selected from a cyano group, a halogen atom, a hydroxyl group, a C₁₋₆ alkyl group, a halo-C₁₋₆ alkyl group and a C₁₋₆ alkoxy group; and a cyclopropane derivative or a pharmaceutically acceptable salt thereof, wherein A₁ represents a phenyl group, a pyrazolyl group or a triazolyl group which may optionally have 1 to 3 substituents selected from a halogen atom, a C₁₋₆ alkyl group, a halo-C₁₋₆ alkyl group, a C₁₋₆ alkoxy group, a C₃₋₈ cycloalkyl group or a C₁₋₆ alkoxy group, are preferable.

Specifically, the cyclopropane derivative of the present invention is preferably selected from the following compounds:
1) N-phenyl-2-[(3,4-dimethoxyphenyl)oxymethyl]-2-phenylcyclopropanecarboxamide,
2) N-methyl-N-phenyl-2-[(3,4-dimethoxyphenyl)oxymethyl]-2-phenylcyclopropanecarboxamide,
3) N-(5-fluoropyridin-2-yl)-2-[(3,4-dimethoxyphenyl)oxymethyl]-2-phenylcyclopropanecarboxamide,
4) (1R,2S)—N-(5-cyanopyridin-2-yl)-2-[(3,4-dimethoxyphenyl)oxymethyl]-2-phenylcyclopropanecarboxamide,
5) N-(4-methyl-1,3-thiazol-2-yl)-2-[(3,4-dimethoxyphenyl)oxymethyl]-2-phenylcyclopropanecarboxamide,
6) N-(5-chloropyridin-2-yl)-2-[(3,4-dimethoxyphenyl)oxymethyl]-2-phenylcyclopropanecarboxamide,
7) N-(3-methyl-1,2,4-thiadiazol-5-yl)-2-[(3,4-dimethoxyphenyl)oxymethyl]-2-phenylcyclopropanecarboxamide,
8) N-(3-methylisoxazol-5-yl)-2-[(3,4-dimethoxyphenyl)oxymethyl]-2-phenylcyclopropanecarboxamide,
9) N-(1,5-dimethyl-1H-pyrazol-3-yl)-2-[(3,4-dimethoxyphenyl)oxymethyl]-2-phenylcyclopropanecarboxamide,
10) N-(5-fluoropyridin-2-yl)-2-[(3,4-dimethoxyphenyl)oxymethyl]-2-phenylcyclopropanecarboxamide,
11) N-(5-chloropyridin-3-yl)-2-[(3,4-dimethoxyphenyl)oxymethyl]-2-phenylcyclopropanecarboxamide,
12) N-(2-methylpyridin-4-yl)-2-[(3,4-dimethoxyphenyl)oxymethyl]-2-phenylcyclopropanecarboxamide,
13) N-(5-chloro-1,3-thiazol-2-yl)-2-[(3,4-dimethoxyphenyl)oxymethyl]-2-phenylcyclopropanecarboxamide,
14) N-(3-chloropyridin-4-yl)-2-[(3,4-dimethoxyphenyl)oxymethyl]-2-phenylcyclopropanecarboxamide,
15) N-(5-methylpyridin-3-yl)-2-[(3,4-dimethoxyphenyl)oxymethyl]-2-phenylcyclopropanecarboxamide,
16) N-(1,3,4-thiadiazol-2-yl)-2-[(3,4-dimethoxyphenyl)oxymethyl]-2-phenylcyclopropanecarboxamide,
17) N-(5-methyl-1,3,4-thiadiazol-2-yl)-2-[3,4-dimethoxyphenyl)oxymethyl]-2-phenylcyclopropanecarboxamide,
18) N-(4-cyanopyridin-2-yl)-2-[(3,4-dimethoxyphenyl)oxymethyl]-2-phenylcyclopropanecarboxamide,
19) N-(2-fluoropyridin-4-yl)-2-[(3,4-dimethoxyphenyl)oxymethyl]-2-phenylcyclopropanecarboxamide,
20) N-(3-methylpyridin-4-yl)-2-[(3,4-dimethoxyphenyl)oxymethyl]-2-phenylcyclopropanecarboxamide,
21) N-(5-methylisoxazol-3-yl)-2-[(3,4-dimethoxyphenyl)oxymethyl]-2-phenylcyclopropanecarboxamide,
22) N-(5-methyl-1,3-thiazol-2-yl)-2-[(3,4-dimethoxyphenyl)oxymethyl]-2-phenylcyclopropanecarboxamide,
23) N-(4-methyl-1,3-thiazol-2-yl)-2-[(3,4-dimethoxyphenyl)oxymethyl]-2-phenylcyclopropanecarboxamide,
24) N-(5-fluoropyridin-3-yl)-2-[(3,4-dimethoxyphenyl)oxymethyl]-2-phenylcyclopropanecarboxamide,
25) N-(3-trifluoromethylpyridin-4-yl)-2-[(3,4-dimethoxyphenyl)oxymethyl]-2-phenylcyclopropanecarboxamide,
26) N-(6-fluoromethylpyridin-3-yl)-2-[3,4-dimethoxyphenyl)oxymethyl]-2-phenylcyclopropanecarboxamide,
27) N-(6-fluoropyridin-2-yl)-2-[(3,4-dimethoxyphenyl)oxymethyl]-2-phenylcyclopropanecarboxamide,
28) N-(4-chloropyridin-2-yl)-2-[(3,4-dimethoxyphenyl)oxymethyl]-2-phenylcyclopropanecarboxamide,
29) N-(5-cyanopyridin-2-yl)-2-[(3,4-dimethoxyphenyl)oxymethyl]-2-phenylcyclopropanecarboxamide,
30) N-(6-chloropyridazin-3-yl)-2-[3,4-dimethoxyphenyl)oxymethyl]-2-phenylcyclopropanecarboxamide,
31) N-(6-cyanopyridin-3-yl)-2-[(3,4-dimethoxyphenyl)oxymethyl]-2-phenylcyclopropanecarboxamide,
32) N-(2-chloropyridin-4-yl)-2-[(3,4-dimethoxyphenyl)oxymethyl]-2-phenylcyclopropanecarboxamide,
33) N-(pyrimidin-4-yl)-2-[(3,4-dimethoxyphenyl)oxymethyl]-2-phenyl cyclopropanecarboxamide,
34) N-(3-methoxyphenyl)-2-[(3,4-dimethoxyphenyl)oxymethyl]-2-phenylcyclopropanecarboxamide,
35) N-[2-(1H-1,2,4-triazol-3-yl)phenyl]-2-[(3,4-dimethoxyphenyl)oxymethyl]-2-phenylcyclopropanecarboxamide,
36) N-(4-methylpyridin-2-yl)-2-[(3,4-dimethoxyphenyl)oxymethyl]-2-phenylcyclopropanecarboxamide,
37) N-(6-methylpyridin-2-yl)-2-[(3,4-dimethoxyphenyl)oxymethyl]-2-phenylcyclopropanecarboxamide,
38) N-(6-chloropyridin-2-yl)-2-[(3,4-dimethoxyphenyl)oxymethyl]-2-phenylcyclopropanecarboxamide,
39) N-(1-ethyl-1H-pyrazol-5-yl)-2-[(3,4-dimethoxyphenyl)oxymethyl]-2-phenylcyclopropanecarboxamide,
40) N-(3-methylisothiazol-5-yl)-2-[(3,4-dimethoxyphenyl)oxymethyl]-2-phenylcyclopropanecarboxamide,
41) (1R,2S)—N-(pyridin-2-yl)-2-[(3,4-dimethoxyphenyl)oxymethyl]-2-phenylcyclopropanecarboxamide,
42) N-(pyridin-3-yl)-2-(3,4-dimethoxyphenyl)oxymethyl]-2-phenylcyclopropanecarboxamide,
43) N-(pyridin-4-yl)-2-[(3,4-dimethoxyphenyl)oxymethyl]-2-phenylcyclopropanecarboxamide,
44) 2-{[(3,4-dimethoxyphenoxy)methyl]-2-phenylcyclopropyl}-5-fluoro-1H-benzimidazole,
45) 3-{2-[(3,4-dimethoxyphenoxy)methyl]-2-phenylcyclopropyl}-5-phenyl-1H-1,2,4-triazole,
46) N-(pyridin-2-yl)-2-(3-methoxyphenyloxymethyl)-2-phenylcyclopropanecarboxamide,
47) N-(pyridin-2-yl)-2-(4-methoxyphenyloxymethyl)-2-phenylcyclopropanecarboxamide,
48) N-(pyridin-2-yl)-2-[(3-methoxy-4-methoxymethylphenyl)oxymethyl]-2-phenylcyclopropanecarboxamide,
49) N-(pyridin-2-yl)-2-(3,4-dimethoxyphenyl)-2-phenyloxymethylcyclopropanecarboxamide,
50) N-(pyridin-2-yl)-2-[(4-methoxy-3-methoxymethylphenyl)oxymethyl]-2-phenylcyclopropanecarboxamide,
51) N-(pyridin-2-yl)-2-(3,4-dimethoxyphenyl)-2-(4-fluorophenyloxymethyl)cyclopropanecarboxamide,
52) N-(5-fluoropyridin-2-yl)-2-(3,4-dimethoxyphenyl)-2-(4-fluorophenyloxymethyl)cyclopropanecarboxamide,
53) N-(5-chloropyridin-2-yl)-2-[(1-methyl-6-oxo-1,6-dihydropyridin-3-yl)oxymethyl]-2-phenylcyclopropanecarboxamide,
54) N-(5-chloropyridin-2-yl)-2-[(1-ethyl-6-oxo-1,6-dihydropyridin-3-yl)oxymethyl]-2-phenylcyclopropanecarboxamide,
55) N-(5-fluoropyridin-2-yl)-2-[(1-ethyl-6-oxo-1,6-dihydropyridin-3-yl)oxymethyl]-2-phenyl cyclopropanecarboxamide,
56) N-(pyridin-2-yl)-2-[(3,4-dimethoxyphenyl)oxymethyl]-2-(pyridin-2-yl)cyclopropanecarboxamide,
57) (1S,2R)—N-(5-cyanopyridin-2-yl)-2-[(1-ethyl-3-methyl-1H-pyrazol-4-yl)oxymethyl]-2-phenylcyclopropanecarboxamide,
58) (1S,2R)—N-(pyridin-2-yl)-2-[(1-ethyl-3-methyl-1H-pyrazol-4-yl)oxymethyl]-2-phenylcyclopropanecarboxamide,
59) (1S,2R)—N-(6-fluoropyridin-3-yl)-2-[(1-ethyl-3-methyl-1H-pyrazol-4-yl)oxymethyl]-2-phenylcyclopropanecarboxamide,
60) (1S,2R)—N-(4-fluorophenyl)-2-[(1-ethyl-3-methyl-1H-pyrazol-4-yl)oxymethyl]-2-phenylcyclopropanecarboxamide,
61) (1S,2R)—N-(5-methoxypyridin-3-yl)-2-[(1-ethyl-3-methyl-1H-pyrazol-4-yl)oxymethyl]-2-phenylcyclopropanecarboxamide,
62) (1S,2R)—N-(2-fluoropyridin-4-yl)-2-[(1-ethyl-3-methyl-1H-pyrazol-4-yl)oxymethyl]-2-phenylcyclopropanecarboxamide,
63) N-(5-cyanopyridin-2-yl)-2-[(1-ethyl-1H-pyrazol-4-yl)oxymethyl]-2-phenylcyclopropanecarboxamide,
64) (1R,2S)-2-[(1-ethyl-3-methyl-1H-pyrazol-4-yl)oxymethyl]-2-(3-fluorophenyl)-N-(pyridin-2-yl)cyclopropanecarboxamide,
65) (1R,2S)-2-[(1-ethyl-3-methyl-1H-pyrazol-4-yl)oxymethyl]-2-(3-fluorophenyl)-N-(5-fluoropyridin-2-yl)cyclopropanecarboxamide,
66) (1R,2S)-2-[(1-ethyl-3-methyl-1H-pyrazol-4-yl)oxymethyl]-2-(3-fluorophenyl)-N-(5-fluoropyridin-3-yl)cyclopropanecarboxamide,
67) (1R,2S)-2-[(1-ethyl-3-methyl-1H-pyrazol-4-yl)oxymethyl]-2-(3-fluorophenyl)-N-(4-fluorophenyl)cyclopropanecarboxamide,
68) (1R,2S)-2-[(1-ethyl-3-methyl-1H-pyrazol-4-yl)oxymethyl]-N-(5-fluoro-4-methoxypyridin-2-yl)-2-(3-fluorophenyl)cyclopropanecarboxamide,
69) (1R,2S)—N-(5-cyanopyridin-2-yl)-2-[(1-ethyl-3-methyl-1H-pyrazol-4-yl)oxymethyl]-2-(3-fluorophenyl)cyclopropanecarboxamide,
70) (1R,2S)-2-[(1-ethyl-3-methyl-1H-pyrazol-4-yl)oxymethyl]-2-(3-fluorophenyl)-N-(2-fluoropyridin-4-yl)cyclopropanecarboxamide,
71) (1R,2S)-2-[(1,3-dimethyl-6-oxo-1,6-dihydropyridazin-4-yl)oxymethyl]-N-(5-methoxypyridin-3-yl)-2-phenylcyclopropanecarboxamide,
72) (1R,2S)-2-[(1,3-dimethyl-6-oxo-1,6-dihydropyridazin-4-yl)oxymethyl]-N-(4-fluorophenyl)-2-phenylcyclopropanecarboxamide,
73) (1R,2S)-2-[(1,3-dimethyl-6-oxo-1,6-dihydropyridazin-4-yl)oxymethyl]-N-(5-fluoro-4-methylpyridin-3-yl)-2-phenylcyclopropanecarboxamide,
74) (1R,2S)—N-(5-chloropyridin-3-yl)-2-[(1,3-dimethyl-6-oxo-1,6-dihydropyridazin-4-yl)oxymethyl]-2-phenylcyclopropanecarboxamide,
75) (1R,2S)-2-[2-(3-difluoromethyl-5-methyl-1H-1,2,4-triazol-1-yl)ethyl]-N-(5-fluoro-4-methylpyridin-2-yl)-2-phenylcyclopropanecarboxamide,
76) (1R,2S)-2-[2-(3-difluoromethyl-5-methyl-1H-1,2,4-triazol-1-yl)ethyl]-N-(5-fluoropyridin-2-yl)-2-phenylcyclopropanecarboxamide,
77) (1R,2S)—N-(5-chloropyridin-2-yl)-2-[2-(3-difluoromethyl-5-methyl-1H-1,2,4-triazol-1-yl)ethyl]-2-phenylcyclopropanecarboxamide,
78) (1R,2S)-2-[2-(3-difluoromethyl-5-methyl-1H-1,2,4-triazol-1-yl)ethyl]-N-(4-fluorophenyl)-2-phenylcyclopropanecarboxamide,
79) (1R,2S)—N-(5-fluoropyridin-2-yl)-2-[2-(3-difluoromethyl-5-methyl-1H-1,2,4-triazol-1-yl)ethyl]-2-phenylcyclopropanecarboxamide,
80) N-(5-fluoropyridin-2-yl)-2-[2-(3-methoxy-4-oxopyridine-1(4H)-yl)ethyl]-2-phenylcyclopropanecarboxamide,
81) 2-[2-(3,5-dimethyl-1H-1,2,4-triazol-1-yl)ethyl]-N-(5-fluoropyridin-2-yl)-2-phenylcyclopropanecarboxamide,
82) N-(5-chloropyridin-2-yl)-2-[2-(3,5-dimethyl-1H-1,2,4-triazol-1-yl)ethyl]-2-phenylcyclopropanecarboxamide,
83) (1R,2S)—N-(5-fluoropyridin-2-yl)-2-[2-(5-methyl-3-trifluoromethyl-1H-1,2,4-triazol-1-yl)ethyl]-2-phenylcyclopropanecarboxamide,
84) (1R,2S)—N-(5-chloropyridin-2-yl)-2-[2-(5-methyl-3-trifluoromethyl-1H-1,2,4-triazol-1-yl)ethyl]-2-phenylcyclopropanecarboxamide,
85) (1R,2S)—N-(5-cyanopyridin-2-yl)-2-[2-(5-methyl-3-trifluoromethyl-1H-1,2,4-triazol-1-yl)ethyl]-2-phenylcyclopropanecarboxamide,
86) (1R,2S)—N-(5-fluoropyridin-2-yl)-2-[2-(3-cyclopropyl-5-methyl-1H-1,2,4-triazol-1-yl)ethyl]-2-phenylcyclopropanecarboxamide,
87) (1R,2S)—N-(5-chloropyridin-2-yl)-2-[2-(3-cyclopropyl-5-methyl-1H-1,2,4-triazol-1-yl)ethyl]-2-phenylcyclopropanecarboxamide,
88) (1R,2S)—N-(5-cyanopyridin-2-yl)-2-[2-(3-cyclopropyl-5-methyl-1H-1,2,4-triazol-1-yl)ethyl]-2-phenylcyclopropanecarboxamide,
89) (1R,2S)—N-(5-fluoropyridin-2-yl)-2-[2-(3-ethyl-5-methyl-1H-1,2,4-triazol-1-yl)ethyl]-2-phenylcyclopropanecarboxamide, and
90) (1R,2S)—N-(5-chloropyridin-2-yl)-2-[2-(3-ethyl-5-methyl-1H-1,2,4-triazol-1-yl)ethyl]-2-phenylcyclopropanecarboxamide,
   or a pharmaceutically acceptable salt thereof.

Next, a method for producing the compound of the formula (I) of the present invention [hereinafter referred to as a compound (I); compounds represented by other formulae will be referred to in the same manner] or a pharmaceutically acceptable salt thereof will be described.

### 1. General Production Method 1:

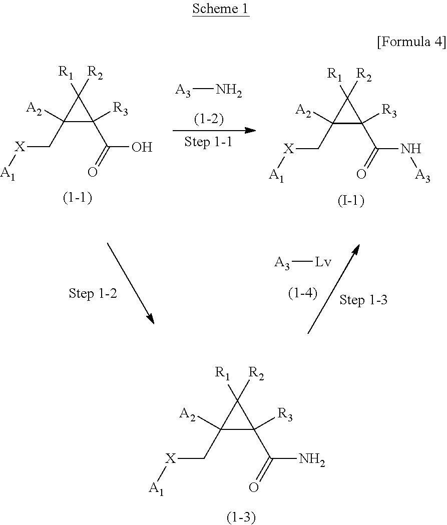

wherein R₁, R₂ and R₃ each represent hydrogen; Lv represents a leaving group including, for example, a halogen atom (a chlorine atom, a bromine atom, an iodine atom, etc.), and a sulfonyloxy group such as a methanesulfonyloxy group, a p-toluenesulfonyloxy group or a trifluoromethanesulfonyloxy group (which is represented by TfO in the formula); and A₁, A₂, A₃ and X have the same meanings as those described above.

### Step 1-1:

The present step is a step of directly condensing the compound (1-1) with the compound (1-2) (method 1), or inducing the compound (1-1) to an acid halide (method 2), a mixed acid anhydride (method 3), an active ester (method 4) or the like and then condensing the obtained product with the compound (1-2), so as to obtain the compound (I-1).

### Method 1:

When the compound (1-1) is directly condensed with the compound (1-2), a condensing agent is used. Such a condensation reaction can be carried out under the same conditions as commonly used conditions described in publications as described below. Known methods are described, for example, in Rosowsky, A.; Forsch, R. A.; Moran, R. G; Freisheim, J. H.; J. Med. Chem., 34(1), 227-234 (1991), Brzostwska, M.; Brossi, A.; Flippen-Anderson, J. L.; Heterocycles, 32(10), 1968-1972 (1991), Romero, D. L.; Morge, R. A.; Biles, C.; Berrios-Pena, N.; May, P. D.; Palmer, J. R.; Johnson, P. D.; Smith, H. W.; Busso, M.; Tan, C.-K.; Voorman, R. L.; Reusser, F.; Althaus, I. W.; Downey, K. M.; So, A. G; Resnick, L.; Tarpley, W. G, Aristoff, P. A.; J. Med. Chem., 37(7), 998-1014 (1994)

The compound (1-2) may be either a free form or a salt.

The solvent used in the present reaction is not particularly limited, as long as it does not inhibit the reaction. Examples of such a solvent include tetrahydrofuran, 1,4-dioxane, ethyl acetate, methyl acetate, dichloromethane, chloroform, N,N-dimethylformamide, toluene and xylene. Examples of a condensing agent include CDI (N,N′-carbonyldiimidazole), Bop (1H-1,2,3-benzotriazol-1-yloxy(tri(dimethylamino))phosphonium hexafluorophosphate), WSC (1-ethyl-3-(3-dimethylaminopropyl)carbodiimide hydrochloride), DCC(N,N-dicyclohexylcarbodiimide), diethylphosphorylcyanide, and PyBOP (benzotriazol-1-yloxytris(pyrrolidino)phosphoniumhexafluorophosphate). The compound (1-2) is used in an amount from 1 equivalent to a largely excessive amount with respect to the compound (1-1). In addition, an organic base such as triethylamine may be added in an amount from 1 equivalent to a largely excessive amount to the compound (1-1), as necessary.

The reaction time is not particularly limited. It is generally from 0.5 to 48 hours, and preferably from 0.5 to 24 hours. The reaction temperature depends on a raw material used, a solvent used, and the like, and thus, it is not particularly limited. It is preferably from an ice cooling temperature to a solvent reflux temperature.

### Method 2: (Synthetic Method Using Acid Halide)

In the present reaction, the compound (1-1) is converted to the corresponding acid halide according to a method known to a person skilled in the art, and the acid halide is then allowed to react with the compound (1-2) to obtain the compound (I-1).

Examples of a base used in the reaction include triethylamine, pyridine, potassium carbonate and diisopropylethylamine. The reaction temperature is not particularly limited. It is generally from −78° C. to a solvent reflux temperature, and preferably from −20° C. to room temperature. The solvent used in the reaction is not particularly limited, as long as it does not inhibit the reaction and is able to dissolve a starting substance to a certain extent. Preferred examples of such a solvent include tetrahydrofuran, ether, toluene and dichloromethane.

### Method 3: (Synthetic Method Using Acid Anhydride)

After the compound (1-1) has been converted to a mixed acid anhydride, the mixed acid anhydride is allowed to react with the compound (1-2), so as to obtain the compound (I-1). The mixed acid anhydride can be synthesized by means known to a person skilled in the art. For example, it can be synthesized by reacting the compound (1-1) with a chloroformic acid ester such as ethyl chloroformate in the presence of a base such as triethylamine. Such a chloroformic acid ester and a base are used in an amount of 1 to 2 equivalents with respect to the compound (1-1). The reaction temperature is from −30° C. to room temperature, and preferably −20° C. to room temperature.

The step of condensing the mixed acid anhydride and the compound (1-2) is carried out, for example, by reacting the mixed acid anhydride with the compound (1-2) in a solvent such as dichloromethane, tetrahydrofuran or N,N-dimethylformamide. The compound (1-2) is used in an amount from 1 equivalent to a largely excessive amount with respect to the mixed acid anhydride.

The reaction time is not particularly limited. It is generally from 0.5 to 48 hours, and preferably from 0.5 to 12 hours. The reaction temperature is from −20° C. to 50° C., and preferably from −20° C. to room temperature.

### Method 4: (Synthetic Method Using Active Ester)

After the compound (1-1) has been converted to an active ester, the active ester is allowed to react with the compound (1-2), so as to obtain the compound (I-1). The step of obtaining the active ester is carried out, for example, by reacting the compound (1-1) with an active ester-synthesizing reagent in a solvent such as 1,4-dioxane, tetrahydrofuran or N,N-dimethylformamide in the presence of a condensing agent such as DCC. An example of the active ester-synthesizing reagent is N-hydroxysuccinimide. Such an active ester-synthesizing reagent and a condensing agent are used in an amount of 1 to 1.5 equivalents with respect to the compound (1-1). The reaction time is not particularly limited. It is generally from 0.5 to 48 hours, and preferably from 0.5 to 24 hours.

The reaction temperature is from −20° C. to −50° C., and preferably from −20° C. to room temperature.

The step of condensing the active ester and the compound (1-2) is carried out, for example, by reacting the active ester with the compound (1-2) in a solvent such as dichloromethane, tetrahydrofuran or N,N-dimethylformamide. The compound (1-2) is used in an amount from 1 equivalent to a largely excessive amount with respect to the active ester. The reaction time is not particularly limited. It is generally from 0.5 to 48 hours, and preferably from 0.5 to 24 hours. The reaction temperature is from −20° C. to −50° C., and preferably from −20° C. to room temperature.

### Step 1-2:

The present step is a step of obtaining the compound (1-3) from the compound (1-2).

The present step is a step of converting the compound (1-1) to the corresponding acid halide or acid anhydride by the methods described in method 2 and method 3 above and then reacting the acid halide or acid anhydride with ammonia, so as to obtain the compound (1-3). The ammonia used in the reaction may be either gas or an aqueous solution. It may also be an ammonia salt. The compound (1-3) can also be produced by reacting hexamethyl disilazane with an acid halide and then adding methanol to the reaction product, followed by an acid treatment (R. Pellegata et al., Synthesis, 1985, 517).

Moreover, the compound (1-3) can also be produced by heating the compound (1-1) and urea.

### Step 1-3:

The present step is a step of obtaining the compound (I-1) from the compound (1-3).

This is a step of subjecting the compound (1-3) and the compound (1-4) to a coupling reaction using a transition metal, so as to obtain the compound (I-1).

In the present step, the reaction can be carried out under conditions that are commonly applied to the coupling reaction between an aryl halide or arylboronic acid and an acid amide, in which a transition metal is used.

A coupling reaction using copper is described, for example, in publications such as Hanhui Xu, Christian Wolf, Chem. Commun, 2009, 1715; and Suribabu Jammi et al., Synlett. 2009 (20), 3323. The type of a copper reagent used in the present reaction is not particularly limited. Preferred examples of such a copper reagent include cuprous iodide, cuprous oxide, and copper(II) trifluoromethanesulfonate.

A coupling reaction using a palladium complex is described, for example, in publications such as Van den Hoogenband, A et al., Tetrahedron Lett. 2004, 45, 8535; and Ghosh, A et al., Org. Lett. 2003, 5, 2207. The type of a palladium reagent used in the present reaction is not particularly limited. Preferred examples of such a palladium reagent include tris(dibenzylideneacetone)dipalladium, palladium chloride, and palladium(II) acetate. Examples of a ligand used in the present reaction include XantPhos (4,5-bis(diphenylphosphino)-9,9-dimethylxanthene), X-Phos (2-dicyclohexylphosphino-2′,4′,6′-triisopropylbiphenyl), BINAP (2,2′-bis(diphenylphosphino)-1,1′-binaphthyl), DPPF (1,1′-bis(diphenylphosphino)ferrocene), and tris(tert-butyloxy)phosphine. The transition metal reagent is used in an amount of approximately 0.001 to 0.1 equivalent with respect to the amount of a raw material. The type of a solvent used in the present reaction is not particularly limited, as long as it does not inhibit the reaction. Preferred examples of such a solvent include benzene, toluene, xylene, N,N-dimethylformamide, 1-methyl-2-pyrrolidone, tetrahydrofuran, 1,4-dioxane, acetonitrile, and propionitrile. The reaction temperature is not particularly limited. It is generally from an ice cooling temperature to a solvent reflux temperature, and preferably from room temperature to a solvent reflux temperature, for example. The reaction time is not particularly limited. It is generally from 0.5 to 48 hours, and preferably from 0.5 to 24 hours.

### General Production Method 2:

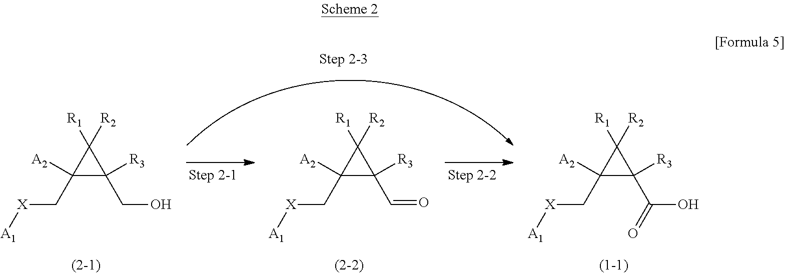

wherein A₁, A₂, R₁, R₂, R₃ and X have the same meanings as those described above.

General production method 2 is a method for producing the compound (1-1) that is a synthetic intermediate of the compound (I-1) according to the present invention, which uses the compound (2-1) as a raw material and involves [step 2-1] and [step 2-2] or [step 2-3].

The compound (2-1) can be produced from a commercially available product by a method known to a person skilled in the art. Further, it can also be produced by applying general production method 3 and general production method 4 described later.

### Step 2-1:

The present step is a step of subjecting the compound (2-1) to an oxidation reaction to obtain the compound (2-2). An aldehyde compound can be obtained from an alcohol compound according to a method known to a person skilled in the art.

Examples of a known oxidation method used in the reaction include Swern oxidation, Corey-Kim oxidation, Moffatt oxidation, PCC oxidation, PDC oxidation, Dess-Martin oxidation, SO₃-pyridine oxidation, and TEMPO oxidation.

The solvent used in the reaction is not particularly limited, as long as it does not inhibit the reaction and dissolves a starting substance to a certain extent. Examples of such a solvent include dimethyl sulfoxide, tetrahydrofuran, toluene, dichloromethane and chloroform.

The reaction temperature is not particularly limited. It is generally from −78° C. to a solvent reflux temperature, and preferably from −78° C. to room temperature. The reaction time is not particularly limited. It is generally from 5 minutes to 48 hours, and preferably from 5 minutes to 24 hours.

### Step 2-2:

The present step is a step of subjecting the compound (2-3) to an oxidation reaction to obtain the compound (1-1). A carboxylic acid compound can be obtained from an aldehyde compound according to a method known to a person skilled in the art.

As an oxidation method, a commonly used oxidation method can be applied. For example, methods described in the Production Examples in Examples can be applied.

### Step 2-3:

The present step is a step of subjecting the compound (2-1) to an oxidation reaction to obtain the compound (1-1). As oxidation conditions, commonly used conditions can be applied. For example, oxidation can be carried out using TEMPO-bisacetyliodobenzene. The solvent used in the reaction is not particularly limited, as long as it does not inhibit the reaction and dissolves a starting substance to a certain extent. For example, dichloromethane, chloroform, acetonitrile or toluene is mixed with water, and the mixed solvent can be used.

The reaction temperature is not particularly limited. It is generally from 0° C. to a solvent reflux temperature. The reaction time is not particularly limited. It is generally from 5 minutes to 48 hours, and preferably from 5 minutes to 24 hours.

Moreover, methods described in the Production Examples in Examples can be applied.

### General Production Method 3:

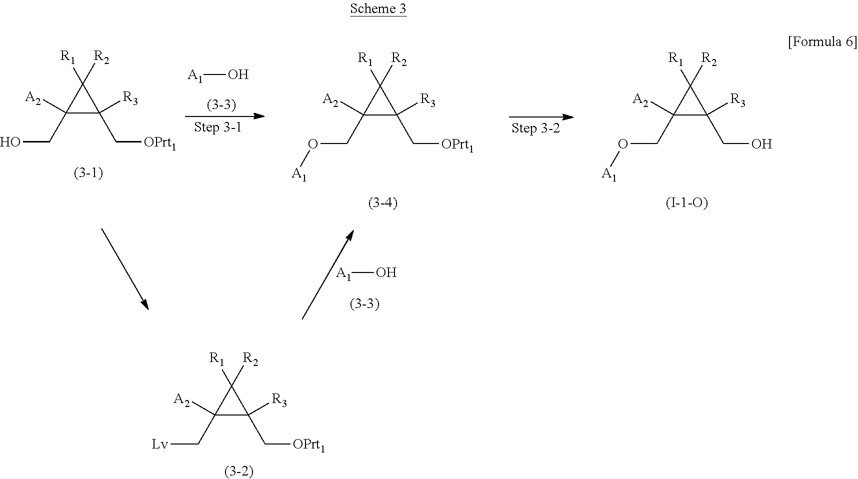

wherein Lv represents a leaving group such as a halogen atom (a chlorine atom, a bromine atom, an iodine atom or the like), a sulfonyloxy group such as a methanesulfonyloxy group, a p-toluenesulfonyloxy group or a trifluoromethanesulfonyloxy group, or the like; Prt₁ represents a protecting group for a hydroxyl group; and A₁, A₂, R₁, R₂ and R₃ have the same meanings as those described above.

General production method 3 is a method for producing the compound (O-1-O) that is a synthetic intermediate of the compound (I) according to the present invention, which uses the compound (3-1) as a raw material and involves [step 3-1] and [step 3-2].

The compound (I-1-O) can also be produced from a commercially available product according to a method known to a person skilled in the art. Further, it can also be produced by applying methods described in the Production Examples in Examples.

### Step 3-1:

The present step is a step of allowing the compound (3-1) to directly react with the compound (3-3), or of converting the compound (3-1) to the compound (3-2) and then allowing the compound (3-2) to react with the compound (3-3), so as to obtain the compound (3-4).

When the compound (3-1) is allowed to directly react with the compound (3-3), the present reaction can be carried out under conditions generally used in the Mitsunobu reaction (for example, conditions described in O. Mitsunobu, Synthesis, 1 (1981), D. L. Hughes, Organic Reactions, 42, 335 (1992), etc.).

The reaction is carried out using a phosphine derivative such as triphenylphosphine and an azodicarboxylic acid diester such as diethyl azodicarboxylate or diisopropyl azodicarboxylate. The solvent used in the reaction is not particularly limited, as long as it does not inhibit the reaction and dissolves a starting substance to a certain extent. For example, tetrahydrofuran, benzene, toluene or N,N-dimethylformamide can be used. The reaction temperature is not particularly limited. It is generally from an ice cooling temperature to room temperature.

Alternatively, the compound (3-4) can be produced by converting the compound (3-1) to the compound (3-2) having a leaving group and then performing a nucleophilic substitution reaction between the compound (3-2) and the compound (3-3). Specifically, a base is allowed to act on the compound (3-3) to form an anion, and the anion is then allowed to react with the compound (3-2), so as to obtain the compound (3-4), for example.

The solvent used in the reaction is not particularly limited, as long as it does not inhibit the reaction. The present reaction can be carried out by allowing a suitable base to act on the compound (3-3), in an amount of 1 equivalent to a largely excessive amount with respect to the compound, in an organic solvent such as diethyl ether, tetrahydrofuran, 1,4-dioxane, N,N-dimethylformamide or dimethyl sulfoxide. Examples of the used base include sodium hydroxide, potassium hydroxide, sodium hydride, potassium hydride, sodium methoxide, sodium ethoxide, and potassium tert-butoxide.

The reaction temperature is not particularly limited. It is generally from −78° C. to a solvent reflux temperature, and preferably from an ice cooling temperature to 100° C.

The compound (3-2) can be produced by converting the hydroxyl group of the compound (3-1) to a leaving group.

Examples of such a leaving group include a halogen atom (a chlorine atom, a bromine atom or an iodine atom), and a sulfonyloxy group such as a methanesulfonyloxy group, a p-toluenesulfonyloxy group or a trifluoromethanesulfonyloxy group.

The reaction can be carried out under the same conditions as those generally used in a reaction of converting the hydroxyl group to such a leaving group (for example, conditions described in R. K. Crossland and K. L. Servis, Journal of Organic Chemistry, 35, 3195 (1970), Y. Yoshida, Y. Sakakura, N. Aso, S. Okada, and Y. Tanabe, Tetrahedron, 55, 2183 (1999).

When the leaving group is a halogen atom, for example, the compound (3-2) can be produced by allowing the compound (3-1) to react with thionyl chloride, thionyl bromide, phosphorus tribromide or tetrahalogenomethane triphenylphosphine. The solvent used in the reaction is not particularly limited, as long as it does not inhibit the reaction and dissolves a starting substance to a certain extent. Preferred examples of such a solvent include benzene, toluene, xylene, dichloromethane and chloroform. Further, there may be a case in which favorable results such as the improvement of a yield can be obtained by addition of a base. The base used in the reaction is not particularly limited, as long as it does not inhibit the reaction. Preferred examples of such a base include sodium carbonate, potassium carbonate, triethylamine, pyridine and diisopropylethylamine The reaction temperature is generally from −78° C. to a solvent reflux temperature, and preferably from an ice cooling temperature to a solvent reflux temperature.

When the leaving group is a sulfonyloxy group, the compound (3-2) can be produced by allowing the compound (3-1) to react with methanesulfonyl chloride, p-toluenesulfonyl chloride, anhydrous trifluoromethanesulfonic acid, etc. The solvent used in the reaction is not particularly limited, as long as it does not inhibit the reaction and dissolves a starting substance to a certain extent. Preferred examples of such a solvent include tetrahydrofuran, toluene, xylene, dichloromethane, chloroform and N,N-dimethylformamide. The reaction temperature is generally from −78° C. to a solvent reflux temperature, and preferably from an ice cooling temperature to room temperature. Further, there may be a case in which favorable results such as the improvement of a yield can be obtained by addition of a base. The base used in the reaction is not particularly limited, as long as it does not inhibit the reaction. Preferred examples of such a base include sodium carbonate, potassium carbonate, triethylamine, pyridine and diisopropylethylamine

### Step 3-2:

The present step is a step of deprotecting the compound (3-4) to obtain the compound (I-1-O).

When Prt₁ is a tert-butyldimethylsilyl group or a tert-butyldiphenylsilyl group, the reaction can be carried out under the same conditions as those generally used in the deprotection reaction of a silyl group (for example, conditions described in publications such as T. W. Green and P. G. M. Wuts, “Protective Groups in Organic Chemistry, Third Edition,” John Wiley & Sons (1999), pp. 113-148). Specifically, tetra-n-butylammonium fluoride is allowed to act on the compound (3-4) in an organic solvent such as tetrahydrofuran, or hydrochloric acid is allowed to act on the compound (3-4) in ethanol, so as to obtain the compound (I-1-O). The solvent used in the present reaction is not particularly limited, as long as it does not inhibit the reaction. Preferred examples of such a solvent include dichloromethane, methanol, ethanol, propanol, ethyl acetate, tetrahydrofuran and 1,4-dioxane. Further, there may be a case in which favorable results such as the improvement of a yield can be obtained by addition of an acetic acid.

When Prt₁ is a benzyl group, the reaction can be carried out under the same conditions as those generally used in the deprotection reaction of a benzyl group (for example, conditions described in publications such as T. W. Green and P. G. M. Wuts, “Protective Groups in Organic Chemistry, Third Edition,” John Wiley & Sons (1999), pp. 76-86). Specifically, the reaction can be carried out, for example, by a catalytic reduction method, which uses palladium-carbon, palladium hydroxide-carbon or the like as a catalyst in an organic solvent such as ethanol in a hydrogen atmosphere.

The solvent used in the present reaction is not particularly limited, as long as it does not inhibit the reaction. Examples of such a solvent include methanol, ethanol, propanol, ethyl acetate, tetrahydrofuran and 1,4-dioxane. The reaction conditions are not particularly limited. The reaction can be carried out at a temperature from room temperature to a solvent reflux temperature at normal atmospheric pressure to 150 atmospheric pressures, and preferably at a temperature from room temperature to 60° C. at normal atmospheric pressure to 5 atmospheric pressures.

### General Production Method 4:

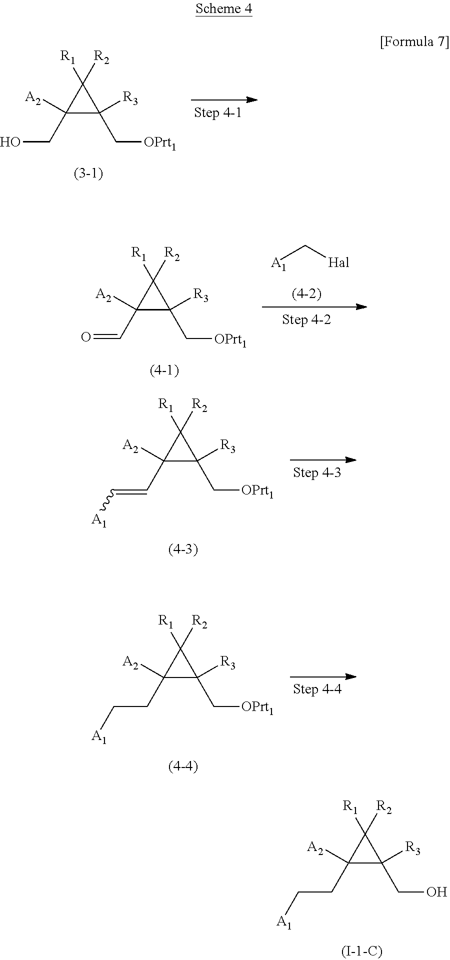

wherein Prt₁, A₁, A₂, R₁, R₂ and R₃ have the same meanings as those described above.

General production method 4 is a method for producing the compound (I-1-C) that is a synthetic intermediate of the compound (I) according to the present invention, which uses the compound (3-1) as a raw material and involves 4 steps from [step 4-1] to [step 4-4].

The compound (3-1) can also be produced from a commercially available product by a method known to a person skilled in the art. Further, it can also be produced by applying the method described in general production method 5 described later.

### Step 4-1

The present step is a step of oxidizing the alcohol of the compound (3-1) to obtain an aldehyde body (4-1). The present reaction can be carried out under the same conditions as those in step 2-1.

### Step 4-2

The present step is a step of obtaining the olefin (4-3) from the aldehyde (4-1). The present reaction can be carried out under commonly used conditions. Specifically, the compound (4-2) and a Wittig reagent synthesized from triphenylphosphine are used for example, and these are allowed to react with the compound (4-1) in the presence of a base, so as to obtain the compound (4-3).

### Step 4-3

The present step is a step of reducing an olefin according to catalytic hydrogen reduction. The present reaction can be carried out under commonly used conditions.

### Step 4-4

The present step is a step of deprotecting the compound (4-3) to obtain the compound (I-1-C). The present reaction can be carried out by the same method as that in step 3-2.

### 5. General Production Method 5:

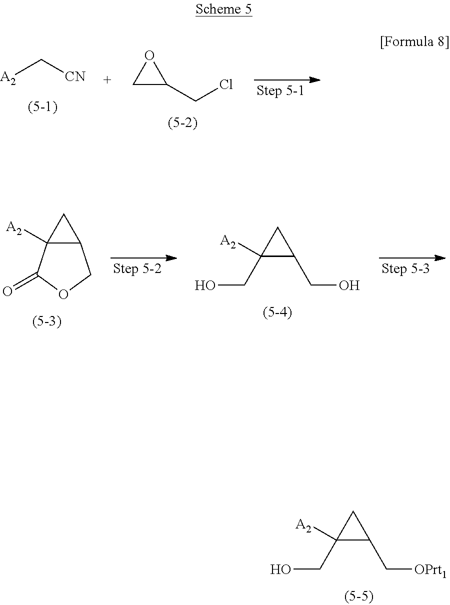

wherein Prt₁ and A₁ have the same meanings as those described above.

General production method 5 is a method for producing the compound (5-5) that is a synthetic intermediate of the compound (I) according to the present invention, which uses the compound (5-1) as a raw material and involves [step 5-1] to [step 5-3].

The compound (5-5) can also be produced from a commercially available product by a method known to a person skilled in the art. Further, it can also be produced by applying methods described in the Production Examples in Examples.

### Step 5-1

The present step is a step of reacting an acetonitrile derivative (5-1) with the epichlorohydrin (5-2) to obtain the compound (5-3). The compound (5-3) can be produced under commonly used reaction conditions (for example, conditions described in S, Shuto, Bioorganic & Medicinal Chemistry, 10 (2002), 3829), or by applying methods described in the Production Examples in Examples. Moreover, an optically active substance of the compound (5-3) can be obtained using an optically active epichlorohydrin.

### Step 5-2

The present step is a step of reducing the lactone (5-3) to obtain the compound (5-4). Examples of a reducing agent used in the reaction include sodium borohydride, lithium borohydride, and lithium aluminum hydride.

The solvent used in the present reaction is not particularly limited, as long as it does not inhibit the reaction and dissolves a starting substance to a certain extent. Examples of such a solvent include tetrahydrofuran and diethyl ether. In some cases, an alcoholic solvent such as methanol is mixed with such a solvent. The reaction temperature is not particularly limited. It is generally from −78° C. to a solvent reflux temperature, and preferably from −78° C. to room temperature. The reaction time is not particularly limited. It is generally from 5 minutes to 48 hours, and preferably from 5 minutes to 24 hours.

### Step 5-3

The present step is a step of protecting the hydroxyl group of the compound (5-4). Examples of a protecting group used herein include an acetyl group, a methoxymethyl group, a trityl group, a benzyl group, a t-butyldiphenylsilyl group, and a triisopropylsilyl group. The present reaction can be carried out under the same conditions as those commonly used in the introduction of a protecting group into a hydroxyl group (for example, conditions described in publications such as T. W. Green and P. G. M. Wuts, “Protective Groups in Organic Chemistry, Third Edition,” John Wiley & Sons (1999), pp. 17-245). In addition, the present reaction can also be carried out by acetylation using an enzyme.

### 6. General Production Method 6:

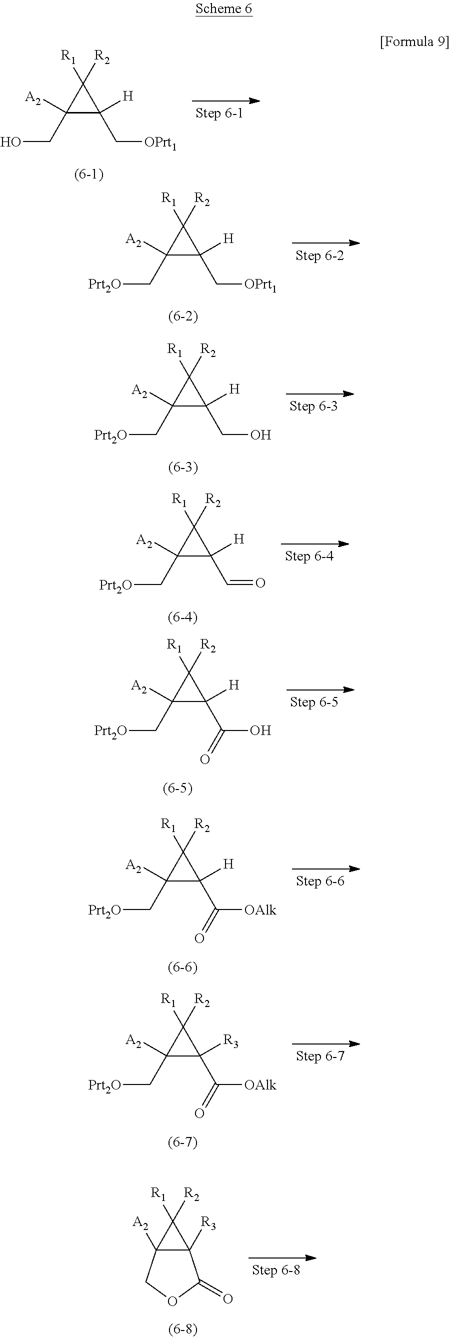

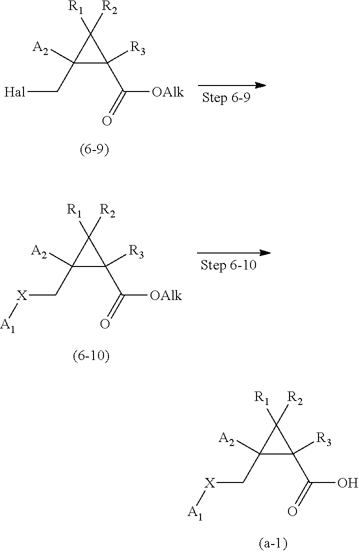

wherein Alk represents a C₁₋₆ alkyl group; Hal represents a halogen atom; Prt₁ represents a silyl group such as a t-butyldimethylsilyl group, a t-butyldiphenylsilyl group or a triisopropylsilyl group; Prt₂ represents a protecting group for a hydroxyl group, other than a silyl group; and X, R₁, R₂, R₃, A₁ and A₂ have the same meanings as those described above. General production method 6 is a method for producing the compound (a-1) that is a synthetic intermediate of the compound (I) according to the present invention, which uses the compound (6-1) as a raw material and involves 10 steps from [step 6-1] to [step 6-10].

### Step 6-1

The present step is a step of protecting the hydroxyl group of the compound (6-1). Examples of a protecting group used herein include a methoxymethyl group, a trityl group and a benzyl group. Such a protecting group can be introduced under commonly used conditions described in step 5-3.

### Step 6-2

The present step is a step of selectively deprotecting the protecting group of the compound (6-2). The deprotection can be carried out under commonly used conditions.

### Steps 6-3, 6-4

The present steps are steps of obtaining the carboxylic acid (6-5) from the compound (6-3) by the same methods as those of step 2-1 and step 2-2 of general production method 2.

### Step 6-5

The present step is a step of esterifying the carboxylic acid (6-5) to obtain the compound (6-6). Esterification can be carried out under commonly used conditions.

### Step 6-6

The present step is a step of introducing the substituent (R3) into the carbonyl a carbon of the ester body (6-6). A preferred example of a base used herein is lithium diisopropylamide. As an alkylating agent, alkyl halide, aldehyde, ketone or the like is used. The solvent used in the reaction is not particularly limited, as long as it does not inhibit the reaction and dissolves a starting substance to a certain extent. Examples of such a solvent include tetrahydrofuran and diethyl ether. The reaction temperature is not particularly limited. It is generally from −78° C. to a solvent reflux temperature, and preferably from −78° C. to room temperature. The reaction time is not particularly limited. It is generally from 5 minutes to 48 hours, and preferably from 5 minutes to 24 hours.

### Step 6-7

The present step is a step of selectively deprotecting the protecting group of the compound (6-7). In general, at the same time of deprotection, cyclization into lactone progresses in a molecule. The deprotection can be carried out under commonly used conditions.

### Step 6-8

The present step is a step of reacting the compound (6-8) with thionyl halide in an alcoholic solvent, so as to obtain the haloester (6-9). The thionyl halide used in the reaction is preferably thionyl bromide. As a solvent, methanol or ethanol is preferable. The reaction temperature is not particularly limited. It is generally from −78° C. to a solvent reflux temperature, and preferably from −78° C. to room temperature. The reaction time is not particularly limited. It is generally from 5 minutes to 48 hours, and preferably from 5 minutes to 48 hours.

### Step 6-9

The present step is a step of obtaining the compound (6-10) as a result of the nucleophilic substitution reaction between the compound (6-9) and the compound (3-3). The reaction conditions may be the same as those for the method for producing the compound (3-4) from the compound (3-2) in general production method 3.

### Step 6-10

The present step is a step of obtaining the compound (a-1) as a result of the ester hydrolysis of the compound (6-10). As reaction conditions, a sodium hydroxide aqueous solution or a potassium hydroxide aqueous solution may be used, for example. Also, an organic solvent such as methanol or ethanol is used, as necessary. The reaction temperature is not particularly limited. It is generally from −78° C. to a solvent reflux temperature, and preferably from room temperature to a solvent reflux temperature. The reaction time is not particularly limited. It is generally from 5 minutes to 48 hours.

### General Production Method 7:

General production method 7 is a method for producing a compound (7-2) that is a synthetic intermediate of the compound (I) according to the present invention, which uses a compound (7-1) as a raw material and involves [step 7-1]. The compound (7-1) can also be produced from a commercially available product by a method known to a person skilled in the art.

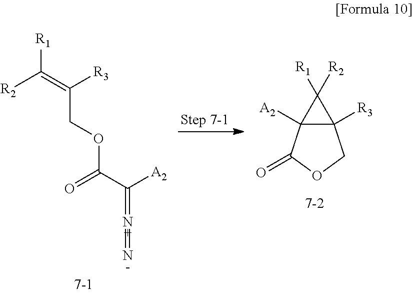

### Step 7-1

The present step is a step of obtaining the compound (7-2), which involves intramolecular cyclization of the diazo compound (7-1). The reaction can be carried out under commonly used conditions for generating carbene from a diazo compound. The reaction can be carried out, for example, by the methods described in Doyle, M, P., Organic Letters 2(8) 1145-; and Chen, C., Bioorganic & Medicinal Chemistry Letters, 18 (2008) 3328-.

### General Production Method 8:

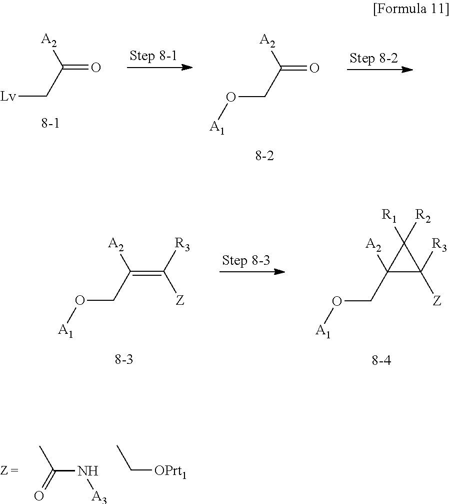

General production method 8 is a method for producing the compound (8-4) from the compound (8-1) via [step 8-1], [step 8-2] and [step 8-3]. The compound (8-1) can be produced from a commercially available product by a method known to a person skilled in the art.

### Step 8-1

The present step is a step of producing the compound (8-2) from the compound (8-1) by applying the method for producing the compound (3-4) from the compound (3-2) in general production method 3.

### Step 8-2

The present step is a step of obtaining the olefin (8-3) from the ketone body (8-2) by the Wittig reaction or the Horner-Wadsworth-Emmons reaction. The present reaction can be carried out under commonly used conditions.

### Step 8-3

The present step is a step of obtaining the compound (8-4) by cyclopropanation of the olefin (8-3). Such cyclopropanation can be carried out, for example, by the Simmons-Smith reaction, or under conditions in which a diazo compound is combined with a metal catalyst such as rhodium acetate.

### General Production Method 9:

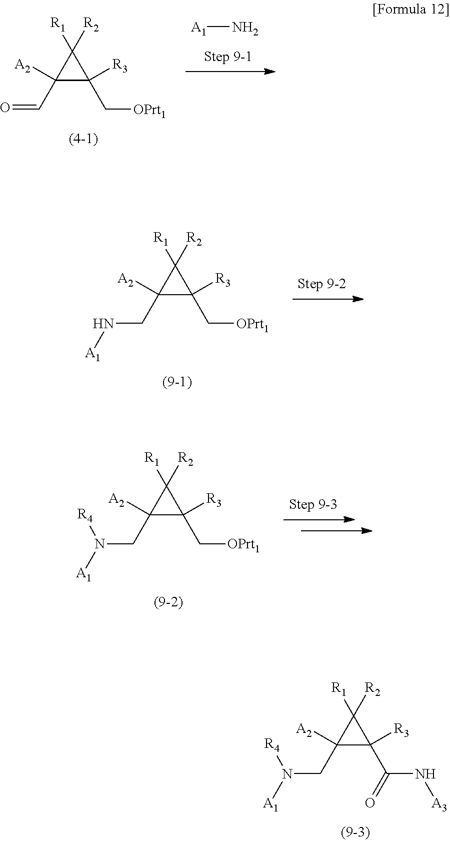

### Step 9-1

The present step is a step of producing the compound (9-1) by reductive amination of the compound (4-1). As reaction conditions, ordinary conditions for reductive amination can be applied. Examples of a reducing agent include sodium borohydride and sodium triacetoxyborohydride.

The solvent used in the reaction is not particularly limited, as long as it does not inhibit the reaction and dissolves a starting substance to a certain extent. Examples of such a solvent include tetrahydrofuran and DMF. In some cases, an acid such as acetic acid may be mixed with such a solvent. The reaction temperature is not particularly limited. It is generally from −78° C. to a solvent reflux temperature, and preferably from 0° C. to room temperature. The reaction time is not particularly limited. It is generally from 5 minutes to 48 hours, and preferably from 5 minutes to 24 hours.

### Step 9-2

The present step is a step of producing the compound (9-2) by reductive amination of the compound (9-1). The reaction conditions are the same as those applied in step 9-1.

### Step 9-3

The present step is a step of producing the compound (9-3) from the compound (9-2) according to the methods described in step 3-2, step 2-1, step 2-2, and general production method 1.

### General Production Method 10:

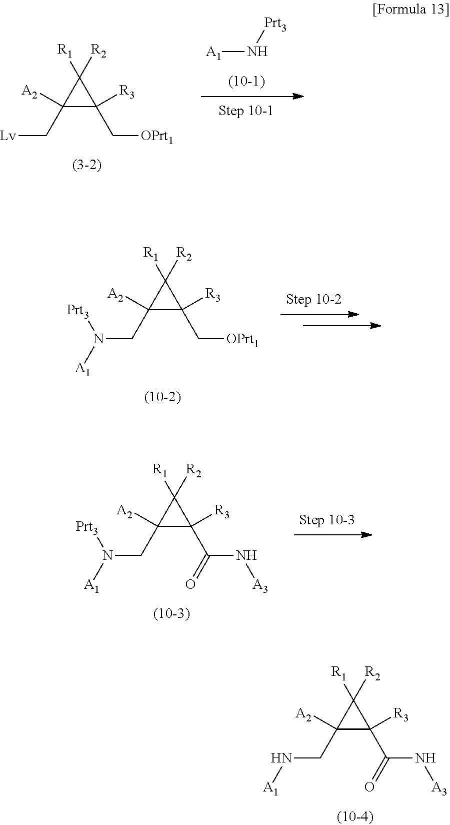

### Step 10-1

The present step is a step of reacting the compound (3-2) with the amine (10-1) protected by an amide or a carbamate in the presence of a base, so as to produce the compound (10-2). Preferred examples of a base used herein include sodium hydride, cesium carbonate, and sodium hydroxide. The solvent used in the reaction is not particularly limited, as long as it does not inhibit the reaction and dissolves a starting substance to a certain extent. Examples of such a solvent include tetrahydrofuran, acetonitrile and DMF. The reaction temperature is not particularly limited. It is generally from 0° C. to a solvent reflux temperature. The reaction time is not particularly limited. It is generally from 5 minutes to 48 hours, and preferably from 5 minutes to 24 hours. In addition, preferred examples of the protecting group Prt₃ include: amide protecting groups such as a trifluoroacetyl group; and carbamate protecting groups such as t-butyl carbamate.

### Step 10-2

The present step is a step of producing the compound (10-3) from the compound (10-2) according to the method described in step 9-3.

### Step 10-3

The present step is a step of producing the compound (10-4) by deprotection of the compound (10-3). The deprotection can be carried out under commonly used conditions.

### General Production Method 11

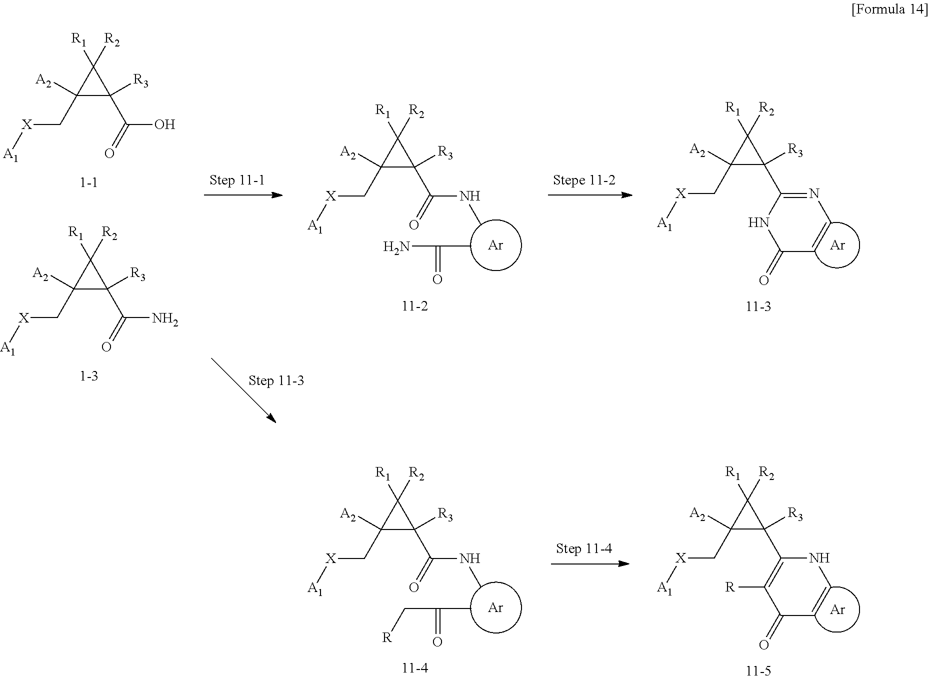

### Step 11-1

The present step is a step of synthesizing the arylamide body (11-2) from the compound (1-1) or the compound (1-3) under the conditions described in general production method 1.

### Step 11-2

The present step is a step of synthesizing the condensed pyrimidone derivative (11-3) from the compound (11-2) by an intramolecular cyclization reaction using a base. Preferred examples of a base used herein include potassium-tert-butoxide, sodium hydride, cesium carbonate, potassium carbonate, and sodium ethoxide. The solvent used in the reaction is not particularly limited, as long as it does not inhibit the reaction and dissolves a starting substance to a certain extent. Examples of such a solvent include tetrahydrofuran, 1,4-dioxane, DMF, MMP, acetonitrile, ethanol, and 2-propanol. The reaction temperature is not particularly limited. It is generally from 0° C. to a solvent reflux temperature, and preferably from room temperature to a solvent reflux temperature. The reaction time is not particularly limited. It is generally from 5 minutes to 48 hours, and preferably from 5 minutes to 24 hours.

### Step 11-3

The present step is a step of synthesizing the arylamide body (11-4) from the compound (1-1) or the compound (1-3) under the conditions described in general production method 1.

### Step 11-4

The present step is a step of synthesizing the condensed pyridone derivative (11-5) from the compound (11-4) by an intramolecular cyclization reaction using a base. Preferred examples of a base used herein include potassium-tert-butoxide, sodium hydride, cesium carbonate, potassium carbonate, and sodium ethoxide. The solvent used in the reaction is not particularly limited, as long as it does not inhibit the reaction and dissolves a starting substance to a certain extent. Examples of such a solvent include tetrahydrofuran, 1,4-dioxane, DMF, NMP, acetonitrile, ethanol and 2-propanol. The reaction temperature is not particularly limited. It is generally from 0° C. to a solvent reflux temperature, and preferably from room temperature to a solvent reflux temperature. The reaction time is not particularly limited. It is generally from 5 minutes to 48 hours, and preferably from 5 minutes to 24 hours.

### General Production Method 12:

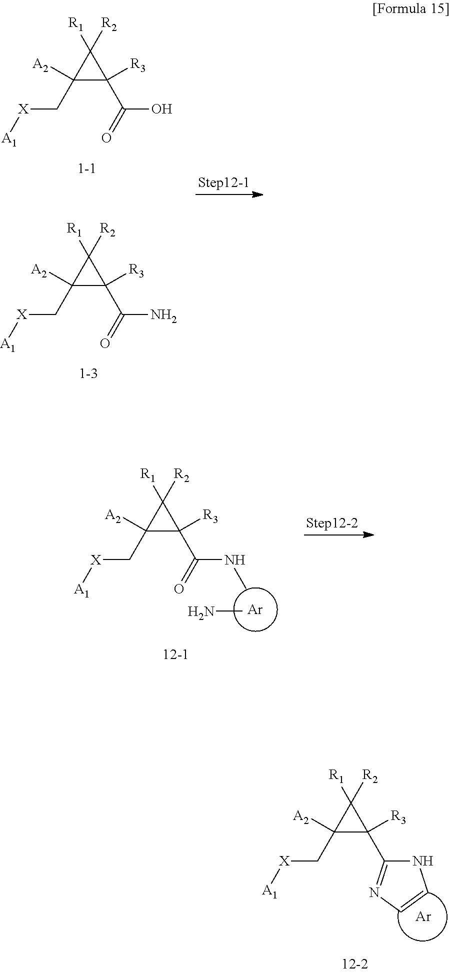

### Step 12-1

The present step is a step of synthesizing the arylamide body (12-1) from the compound (I-1) or the compound (1-3) under the conditions described in general production method 1.

### Step 12-2

The present step is a step of synthesizing the condensed imidazole derivative (12-2) from the compound (12-1) by an intramolecular cyclization reaction using an acid. Preferred examples of an acid used herein include acetic acid, trifluoroacetic acid, hydrochloric acid, and p-toluenesulfonic acid. The solvent used in the reaction is not particularly limited, as long as it does not inhibit the reaction and dissolves a starting substance to a certain extent. For example, acetic acid is used as a solvent. Other examples of a solvent include tetrahydrofuran, 1,4-dioxane, DMF, NMP, acetonitrile, ethanol, and 2-propanol. The reaction temperature is not particularly limited. It is generally from 0° C. to a solvent reflux temperature, and preferably from room temperature to a solvent reflux temperature. The reaction time is not particularly limited. It is generally from 5 minutes to 48 hours, and preferably from 5 minutes to 24 hours.

### General Production Method 13:

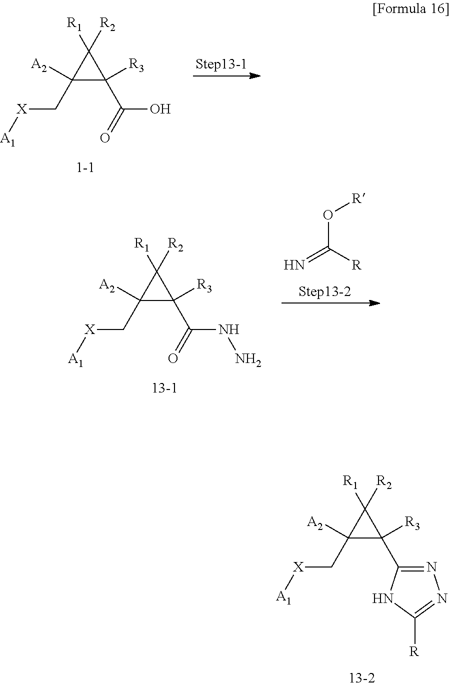

### Step 13-1

The present step is a step of synthesizing the hydrazide (13-1) from the compound (1-1). As synthetic conditions used herein, a generally known method can be applied. For example, mono-protected hydrazine and the compound (1-1) are subjected to amide condensation, and then deprotection is carried out, so as to synthesize the aforementioned compound. The amidation can be carried out by the method described in the step (1-1). The protecting group of hydrazine is not particularly limited. Examples of such a protecting group include tert-butoxycarbonyl, benzyloxycarbonyl, and trifluoroacetyl.

### Step 13-2

The present step is a step of reacting the compound (13-1) with an imidate derivative to synthesize the triazole derivative (13-2). The reaction can be carried out under neutral conditions, or by adding an acid or a base. As an acid used herein, acetic acid, hydrochloric acid or the like is appropriate. As a base used herein, imidazole, triethylamine, potassium carbonate or the like is appropriate. The solvent used in the reaction is not particularly limited, as long as it does not inhibit the reaction and dissolves a starting substance to a certain extent. For example, acetic acid is used as a solvent. Other examples of a solvent include tetrahydrofuran, 1,4-dioxane, DMF, NMP, acetonitrile, ethanol, and 2-propanol. The reaction temperature is not particularly limited. It is generally from 0° C. to a solvent reflux temperature, and preferably from room temperature to a solvent reflux temperature. The reaction time is not particularly limited. It is generally from 5 minutes to 48 hours, and preferably from 5 minutes to 24 hours.

The thus obtained compound of the formula (I) of the present invention can be processed into a pharmaceutically acceptable salt according to an ordinary method, as necessary. Such a pharmaceutically acceptable salt can be produced by appropriately combining methods that are commonly used in the field of organic synthetic chemistry. Specifically, a solution of a free form of the compound of the present invention is subjected to neutralization titration with an acid solution, for example. In addition, the compound of the formula (I) of the present invention is subjected to a well-known solvate formation reaction, as necessary, so that it can be converted to a solvate.

These methods are typical examples of the method for producing the compound (I). The raw material compounds or various reagents in the method for producing the compound (I) may form a salt or a hydrate, and all of them are different depending on a starting material, a solvent used, and the like and are not particularly limited, as long as they do not inhibit the reaction. The solvent used is also different depending on a starting material, a reagent, and the like and, needless to say, is not particularly limited, as long as it does not inhibit the reaction and is able to dissolve a starting substance to a certain extent. When the compound (I) is obtained as a free form, it can be converted, according to an ordinary method, to a state of the aforementioned salt that may be formed by the compound (I). Likewise, when the compound (I) is obtained as a salt of the compound (I), it can be converted to a free form of the compound (I) according to an ordinary method. A free form of the compound (I) or a salt of the compound (I) can be converted to a solvate of the compound (I) according to an ordinary method. Also, various isomers (for example, geometric isomers, optical isomers based on asymmetric carbon atoms, rotational isomers and steric isomers) obtained for the compound (I) can be purified and isolated by using ordinary separation means, for example, recrystallization, diastereomeric salt method, enzymatic resolution method and various chromatography techniques (for example, thin-layer chromatography, column chromatography and gas chromatography).

The term “composition” used herein includes a product comprising a particular ingredient in a particular amount and any product directly or indirectly brought about by the combination of particular ingredients in particular amounts. Such a term related to the pharmaceutical composition is intended to include a product comprising an active ingredient and an inert ingredient constituting a carrier and include every product directly or indirectly brought about by the combination, complexation or aggregation of any two or more ingredients or the dissociation, other kinds of reactions or interaction of one or more ingredients. Thus, the pharmaceutical composition of the present invention includes every composition prepared by mixing the compound of the present invention with a pharmaceutically acceptable carrier. The term “pharmaceutically acceptable” is used to mean that a carrier, a diluent or a vehicle must be compatible with other ingredients of a preparation and must be nontoxic to a taker.

As the ability of the compound of the present invention to bind to orexin receptors OX1R and/or OX2R, antagonism with respect to an orexin 1 receptor and/or an orexin 2 receptor mostly exhibits an IC50 value of 200 nM or lower, and a compound that exhibits an IC50 value of 100 nM or lower is preferable. A cyclopropane compound is thought to be more preferable, in which the ability to bind to an orexin 2 receptor (IC50 value) is 10 nM or lower.

The cyclopropane derivative according to the present invention or a pharmaceutically acceptable salt thereof, or a solvate thereof has orexin receptor antagonism. Thus, the cyclopropane compound according to the present invention or a pharmaceutically acceptable salt thereof, or a solvate thereof has applicability as a therapeutic agent for sleep disorder for which orexin receptor antagonism is effective. Examples of the sleep disorder for which orexin receptor antagonism is effective include insomnia.

The cyclopropane derivative in this invention, a pharmaceutically acceptable salt thereof or a solvate thereof can be used to formulate a preparation according to an ordinary method. Examples of a preferred dosage form include oral preparations (tablets, granules, powders, capsules, syrups etc.), injections (for intravenous administration, for intramuscular administration, for subcutaneous administration, for intraperitoneal administration etc.), or topical products [transdermal absorptions (ointments, adhesive skin patch etc.), ophthalmic solutions, nasal preparations, suppositories etc.].

In the case of manufacturing oral solid preparations, for example, the cyclopropane compound in this invention, a pharmaceutically acceptable salt thereof or a solvate thereof is mixed with excipients, binders, disintegrators, lubricants, coloring agents etc., if necessary, and the obtained mixture is then processed into powders, fine granules, granules, tablets, coated tablets, capsules, etc. according to an ordinary method. In the case of production of tablets or granules, it may be coated with film, if necessary.

Examples of excipients used herein include lactose, corn starch and crystalline cellulose etc. Examples of binders used herein include hydroxypropyl cellulose, hydroxypropylmethyl cellulose etc. Examples of disintegrators used herein include calcium carboxymethyl cellulose, sodium croscarmellose etc. Examples of lubricants used herein include magnesium stearate, calcium stearate etc. Examples of coloring agents used herein include titanium oxide etc. Examples of coating agents used herein include hydroxypropyl cellulose, hydroxypropylmethyl cellulose, methyl cellulose etc. However, needless to say, examples of above agents are not limited thereto.

The aforementioned solid preparation such as tablets, capsules, granules or powders may comprise, as an active ingredient, the cyclopropane compound in this invention, a pharmaceutically acceptable salt thereof or a solvate thereof, in an amount of generally 0.001% to 99.5% by weight, and preferably 0.001% to 90% by weight.

In the case of manufacturing injections (for intravenous administration, for intramuscular administration, for subcutaneous administration, for intraperitoneal administration etc.), for example, pH adjusters, buffering agents, suspending agents, solubilizers, antioxidants, preventing agents (preservatives), tonicity agents, etc. are added to the cyclopropane compound in this invention, a pharmaceutically acceptable salt thereof or a solvate thereof, if necessary and the obtained mixture is then processed into such an injection according to an ordinary method. In addition, such an injection may be prepared as lyophilized preparation for dissolving when used.

Examples of pH adjusters and buffering agents used herein include organic acid or inorganic acid and/or a salt thereof. Examples of suspending agents used herein include methyl cellulose, polysolbate 80, sodium carboxymethyl cellulose, etc. Examples of solubilizers used herein include polysolbate 80, polyethylene solbitan monolaurate, etc. Examples of antioxidants used herein include α-tocopherol, etc. Examples of preventing agents used herein include methyl p-oxybenzoate, ethyl p-oxybenzoate, etc. Examples of tonicity agents used herein include glucose, sodium chloride, mannitol, etc. However, needless to say, examples of above agents are not limited thereto.

Such injection solutions may comprise an active ingredient in an amount of generally 0.000001% to 99.5% by weight, and preferably 0.000001% to 90% by weight.

In the case of manufacturing topical products, for example, the cyclopropane compound I this invention, a pharmaceutically acceptable salt thereof or a solvate thereof is mixed with base materials and the aforementioned adjuvants such as preventing agents, stabilizers, pH adjusters, antioxidants, coloring agents, etc. are added if necessary thereto and the obtained mixture is then processed into transdermal absorptions (ointments, adhesive skin patches, etc.), ophthalmic solutions, nasal preparations, suppositories, etc. according to an ordinary method.

As base materials used herein, various types of raw materials, which are generally used in pharmaceutical products, quasi drugs, cosmetic products, and other products, can be used. Examples of such raw materials include animal or vegetable oils, mineral oils, ester oils, waxes, emulsifiers, higher alcohols, fatty acids, silicon oils, surfactants, phospholipids, alcohols, polyhydric alcohols, water-soluble polymers, clay minerals, purified water etc.

Such external preparations may comprise an active ingredient in an amount of generally 0.000001% to 99.5% by weight, and preferably 0.000001% to 90% by weight.

The dose of the cyclopropane compound according to the present invention, a pharmaceutically acceptable salt thereof or a solvate thereof is different depending on the degree of symptoms, age, sex, body weight, administration route/the type of a salt, the specific type of disease, and the like. In general, in the case of oral administration, the cyclopropane compound according to the present invention, a pharmaceutically acceptable salt thereof or a solvate thereof is administered at a dose of approximately 30 μg to 10 g, preferably 100 μg to 5 g, and more preferably 100 μg to 1 g per adult per day. In the case of administration via injection, it is administered at a dose of approximately 30 μg to 1 g, preferably 100 μg to 500 mg, and more preferably 100 μg to 300 mg per adult per day. In both cases, it is administered once or divided over several administrations.

The compound of the present invention can be used as a chemical probe for capturing a target protein of a physiologically active low-molecular-weight compound. That is to say, the compound of the present invention can be converted to an affinity chromatography probe, a photoaffinity probe or the like, by introducing a labeling group, a linker or the like into a portion other than a structural portion essential for the expression of the activity of the compound according to the methods described in J. Mass Spectrum. Soc. Jpn. Vol. 51, No. 5, 2003, pp. 492-498; WO2007/139149; etc.

Examples of such a labeling group, a linker or the like used for such a chemical probe include groups described in the following groups (1) to (5).

(1) Protein labeling groups, such as photoaffinity labeling groups (for example, a benzoyl group, a benzophenone group, an azide group, a carbonyl azide group, a diaziridine group, an enone group, a diazo group, and a nitro group), and chemical affinity groups (for example, a ketone group in which the alpha carbon atom is replaced with a halogen atom, a carbamoyl group, an ester group, an alkylthio group, a Michael acceptor such as α,β-unsaturated ketone or ester, and an oxirane group),

(2) Cleavable linkers such as —S—S—, —O—Si—O—, monosaccharide (a glucose group, a galactose group, etc.) or disaccharide (lactose, etc.), and oligopeptide linkers that can be cleaved by an enzyme reaction,

(3) Fishing tag groups such as biotin and a 3-(4,4-difluoro-5,7-dimethyl-4H-3a,4a-diaza-4-bora-s-indacen-3-yl)propionyl group,

(4) Radioactive labeling groups such as ¹²⁵I, ³²P, ³H and ¹⁴C; fluorescent labeling groups such as fluorescein, rhodamine, dansyl, umbelliferone, 7-nitrofurazanyl, and 3-(4,4-difluoro-5,7-dimethyl-4H-3a,4a-diaza-4-bora-s-indacen-3-yl)propionyl group; chemiluminescent groups such as lumiferin and luminol; and detectable markers including heavy metal ions such as a lanthanoid metal ion and a radium ion, and

(5) Groups that are allowed to bind to solid-phase carriers, such as glass beads, a glass bed, a microtiter plate, agarose beads, an agarose bed, polystyrene beads, a polystyrene bed, nylon beads and a nylon bed.

A probe, which is prepared by introducing a labeling group or the like selected from the above described groups (1) to (5) into the compound of the present invention according to the methods described in the aforementioned publications and the like, can be used as a chemical probe for identifying a labeled protein useful for the search of a novel target of drug discovery.

Hereinafter, the present invention will be described more in detail in Examples, Production Examples and test examples. However, these examples are not intended to limit the scope of the present invention. Moreover, abbreviations used in Examples are commonly used abbreviations that are well known to a person skilled in the art. Several abbreviations are as follows.
DMSO: dimethyl sulfoxide
THF: tetrahydrofuran
DMF: N,N-dimethylformamide
TFA trifluoroacetic acid
NMP: 1-methyl-2-pyrrolidinone
NaHMDS: sodium hexamethyldisilazide
WSC: 1-ethyl-3-(3-dimethylaminopropyl)carbodiimide hydrochloride
Xantphos: 4,5-bis(diphemylphosphino)-9,9-dimethylxanthene
HATU: O-(7-azabenzotriazol-1-yl)-N,N,N′,N′-tetramethyluronium hexafluorophosphate
HBTU: O-benzotriazol-1-yl-N,N,N,N′-tetramethyluronium hexafluorophosphate
pTLC: preparatory thin-layer chromatography
LC-MS: liquid chromatography-mass spectrometry
PyBOP: benzotriazol-1-yloxytris(pyrrolidino)phosphonium hexafluorophosphate
Pd₂ DBA₃: tris(dibenzylideneacetone)dipalladium
Pd(t-Bu₃P)₂: bis(tri-t-butylphosphine)palladium

Chemical shifts in proton nuclear magnetic resonance spectrum are recorded by δ unit (ppm) with respect to tetramethylsilane. Coupling coefficients are recorded by hertz (Hz). With regard to pattern, s: singlet, d: doublet, t: triplet, q: quartette, and br: broad.

The term “room temperature” generally means approximately 10° C. to approximately 35° C. in the following Examples and Production Examples. The symbol “%” means percent by weight, unless otherwise specified.

### Production Example 1

### Synthesis of 2-[3,4-dimethoxyphenyl)oxymethyl]-2-phenylcyclopropanecarboxylic acid (Prep 1)

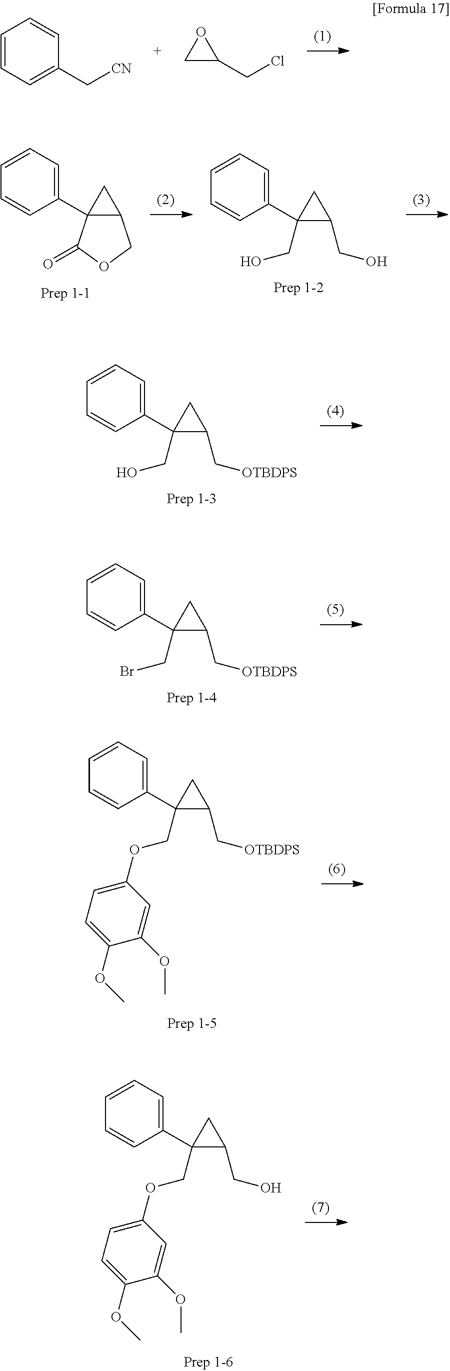

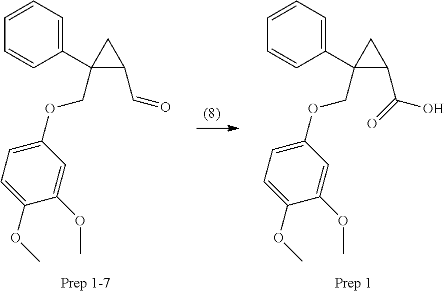

### (1) 1-Phenyl-3-oxabicyclo[3.1.0]hexan-2-one (Prep 1-1)

A benzene solution (50 ml) of phenyl acetonitrile (23.1 ml) was slowly added dropwise in a benzene suspension solution (250 ml) of NaNH₂ (17.2 g) while cooling in an ice bath. The temperature of the reaction solution was returned to room temperature and the reaction solution was stirred for 3 hours and again cooled in an ice bath. To this, epichlorohydrin (15.6 ml) was added dropwise. After the dropwise addition, the temperature of the reaction solution was slowly returned to room temperature and the reaction solution was stirred further for 4 hours. The reaction solution was cooled on ice and a small amount of water was added dropwise. The reaction solution was concentrated under reduced pressure and ethanol (200 ml) and a 1 N potassium hydroxide aqueous solution (100 ml) were added to the residue. The reaction solution was heated to reflux for 16.5 hours. After the temperature of the reaction solution was returned to room temperature, 12 N hydrochloric acid was added up to pH 1. After the reaction solution was concentrated under reduced pressure, ethyl acetate and water were added to carry out liquid separation. The organic layer was successively washed with a saturated sodium bicarbonate aqueous solution and a saturated saline. The organic layer was dried over magnesium sulfate and the solvent was distilled away under reduced pressure. The residue was purified by silica gel column chromatography (n-heptane: ethyl acetate) to obtain the title compound (14.5 g).

¹H-NMR (400 MHz, CDCl₃) δ (ppm): 1.36 (t, J=4.8 Hz, 1H), 1.64 (dd, J=7.6, 4.8 Hz, 1H), 2.53-2.57 (m, 1H), 4.27 (d, J=9.2 Hz, 1H), 4.45 (dd, J=9.2, 4.8 Hz, 1H), 7.24-7.36 (m, 3H), 7.39-7.42 (m, 2H).

### (2) (2-Hydroxymethyl-1-phenylcyclopropyl)methanol (Prep 1-2)

To a THF-methanol solution (30 ml-15 ml) of compound Prep 1-1 (3.49 g), sodium borohydride (1.5 g) was added and the obtained mixture was stirred at room temperature for 16.5 hours. Under cooling on ice, water and 5 N hydrochloric acid were added to the reaction solution, which was extracted with ethyl acetate. The organic layer was washed with a saturated saline, then dried over magnesium sulfate and the solvent was distilled away under reduced pressure. The residue was purified by silica gel column chromatography (n-heptane:ethyl acetate) to obtain the title compound (3.23 g).

¹H-NMR (400 MHz, CDCl₃) δ (ppm): 0.77 (t, J=5.2 Hz, 1H), 1.08 (dd, J=8.8, 5.2, 1H), 1.65-1.72 (m, 1H), 2.65 (t, J=5.6 Hz, 1H), 3.12 (dd, J=8.4, 2.8 Hz, 1H), 3.42 (td, J=12.0, 2.8 Hz, 1H), 3.54 (dd, J=12.0, 5.2 Hz, 1H), 4.10-4.20 (m, 2H), 7.20-7.25 (m, 1H), 7.28-7.33 (m, 2H), 7.37-7.40 (m, 1H).

### (3) [2-(tert-Butyldiphenylsilyloxymethyl)-1-phenylcyclopropyl]methanol (Prep 1-3)

Compound Prep 1-2 (1.87 g) and imidazole (525 mg) were dissolved in DMF (20 ml) and cooled to −15° C. and then tert-butyldiphenylsilyl chloride (2.73 ml) was added dropwise (added dropwise over approximately 10 minutes). After the reaction solution had been stirred for 1 hour, methanol was added to the reaction solution. The solvent was distilled away under reduced pressure, ethyl acetate and water were added thereto and the organic layer was separated. The organic layer was washed with a saturated saline, then dried over magnesium sulfate and filtered. The filtrate was concentrated under reduced pressure and the residue was purified by silica gel column chromatography (n-heptane: ethyl acetate) to obtain the title compound (3.68 g).

¹H-NMR (400 MHz, CDCl₃) δ (ppm): 0.71 (dd, J=5.6 Hz, 5.2 Hz, 1H), 1.04 (dd, J=8.4, 5.2 Hz, 1H), 1.09 (s, 9H), 1.50-1.58 (m, 1H), 3.47 (dd, J=11.6 Hz, 1.6 Hz, 1H), 3.56 (t, J=11.6 Hz, 1H), 3.70 (dd, J=11, 6.1.6 Hz, 1H), 4.10 (t, J=11.6.0 Hz, 1H), 4.19 (dd, J=11.6, 5.2 Hz, 1H), 7.22-7.48 (m, 11H), 7.69-7.75 (m, 4H).

### (4) 1-Bromomethyl-2-(tert-butyldiphenylsilyloxymethyl)-1-phenylcyclopropane (Prep 1-4)

To a dichloromethane solution (50 ml) of compound Prep 1-3 (3.33 g) triphenylphosphine (2.52 g) and carbon tetrabromide (3.98 g) were added at 0° C. and the obtained mixture was stirred for 2 hours in the same condition. To the reaction solution, water was added and the obtained mixture was extracted with ethyl acetate. The organic layer was washed with a saturated saline and then dried over magnesium sulfate. The solvent was distilled away under reduced pressure and the residue was purified by silica gel column chromatography (n-heptane: ethyl acetate) to obtain the title compound (3.45 g).

¹H-NMR (400 MHz, CDCl₃) δ (ppm): 0.92 (dd, J=6.0, 5.2 Hz, 1H), 1.09 (s, 9H), 1.26 (dd, J=8.8, 5.2 Hz, 1H), 1.68-1.75 (m, 1H), 3.70 (d, J=10.4 Hz, 1H), 3.46 (dd, J=11.6, 8.0 Hz, 1H), 3.85 (dd, J=9.6, 1.6 Hz, 1H), 4.08 (dd, J=11.6, 5.6 Hz, 1H), 7.22-7.47 (m, 11H), 7.69-7.73 (m, 4H).

### (5) 2-(tert-Butyldiphenylsilyloxymethyl)-1-(3,4-dimethoxyphenyl)oxymethyl-1-phenylcyclopropane (Prep 1-5)

Compound Prep 1-4 (2.4 g), 3,4-dimethoxyphenol (1.93 g) and potassium carbonate (2.07 g) were suspended in DMF (20 ml) and heated at 70° C. for 24.5 hours. After the temperature of the reaction solution was returned to room temperature, ethyl acetate and water were added to carry out liquid separation. The organic layer was washed with a saturated saline and dried over magnesium sulfate. The solvent was distilled away under reduced pressure and the residue was purified by silica gel column chromatography (n-heptane: ethyl acetate) to obtain the title compound (1.96 g).

¹H-NMR (400 MHz, CDCl₃) δ (ppm): 0.95 (dd, J=6.0, 5.2 Hz, 1H), 1.09 (s, 9H), 1.20 (dd, J=8.8, 5.2 Hz, 1H), 1.58-1.66 (m, 1H), 3.77 (s, 3H), 3.82 (s, 3H), 3.84 (dd, J=8.4, 4.0 Hz, 1H), 4.02 (dd, J=11.6, 6.0 Hz, 1H), 4.10 (d, J=9.6 Hz, 1H), 4.18 (d, J=9.6 Hz, 1H), 6.26 (dd, J=8.4, 2.8 Hz, 1H), 6.40 (d, J=2.8 Hz, 1H), 6.71 (d, J=8.4 Hz, 1H), 7.19-7.45 (m, 11H), 7.66-7.70 (m, 4H).

### (6) 2-Hydroxymethyl-1-(3,4-dimethoxyphenyl)oxymethyl-1-phenylcyclopropane (Prep 1-6)

Compound Prep 1-5 (1.66 g) was dissolved in THF (10 ml) and tetrabutylammonium fluoride (1 M THF solution: 4.5 ml) was added dropwise at room temperature and the obtained mixture was stirred at room temperature for 1 hour. To the reaction solution, water was added and the obtained mixture was extracted with ethyl acetate. The organic layer was washed with a saturated saline and then dried over magnesium sulfate. The solvent was distilled away under reduced pressure and the residue was purified by silica gel column chromatography (n-heptane: ethyl acetate) to obtain the title compound (890 mg).

¹H-NMR (400 MHz, CDCl₃) δ (ppm): 0.92 (dd, J=6.0, 5.2 Hz, 1H), 1.25 (dd, J=8.8, 5.2 Hz, 1H), 1.78-1.85 (m, 1H), 2.86 (d, J=10.8 Hz, 1H), 3.46 (dd, J=12.0, 10.8 Hz, 1H), 3.81 (s, 3H), 3.82 (s, 3H), 3.91 (d, J=10.4 Hz, 1H), 4.07-4.13 (m, 1H), 4.52 (d, J=10.4 Hz, 1H), 6.36 (dd, J=8.8, 3.2 Hz, 1H), 6.44 (d, J=3.2 Hz, 1H), 6.73 (d, J=8.8 Hz, 1H), 7.20-7.25 (m, 1H), 7.26-7.34 (m, 2H), 7.40-7.43 (m, 2H).

### (7) 2-(3,4-Dimethoxyphenyl)oxymethyl-2-phenylcyclopropanecarbaldehyde (Prep 1-7)

A dichloromethane solution (4 ml) of oxalyl chloride (309 μl) was cooled to −78° C. To this, a dichloromethane solution (1 ml) of DMSO (511 μl) was added dropwise (the internal temperature: −60° C. or less). After the reaction solution had been stirred at the same temperature as described above for 30 minutes, a dichloromethane solution (5 ml) of compound Prep 1-6 (566 mg) was added dropwise to the reaction solution at −78° C. and the obtained mixture was stirred at the same temperature as described above for 1.5 hours. To the reaction solution, triethylamine (2.01 ml) was added and the obtained mixture was stirred for 15 minutes. Subsequently, the temperature of the reaction solution was increased to room temperature. To the reaction solution, water was added and the obtained mixture was extracted with dichloromethane. The organic layer was washed with a saturated saline and then dried over magnesium sulfate. The solvent was distilled away under reduced pressure and the residue was purified by silica gel column chromatography (n-heptane: ethyl acetate) to obtain the title compound (558 mg).

¹H-NMR (400 MHz, CDCl₃) δ (ppm): 1.67 (dd, J=8.0, 5.2 Hz, 1H), 1.96 (dd, J=5.2, 5.2 Hz, 1H), 2.37-2.42 (m, 1H), 3.80 (s, 3H), 3.82 (s, 3H), 4.08 (d, J=10.0 Hz, 1H), 4.42 (d, J=10.0 Hz, 1H), 6.29 (dd, J=8.4, 2.8 Hz, 1H), 6.42 (d, J=2.8 Hz, 1H), 6.71 (d, J=8.4 Hz, 1H), 7.26-7.29 (m, 1H), 7.32-7.36 (m, 2H), 7.43-7.45 (m, 2H), 9.70 (d, J=4.4 Hz, 1H).

### (8) 2-(3,4-Dimethoxyphenyl)oxymethyl-2-phenylcyclopropanecarboxylic acid (Prep 1)

Compound Prep 1-7 (342 mg), 2-methyl-2-butene (583 μl) and sodium dihydrogen phosphate (132 mg) were dissolved in a mixed solvent of acetone and water (10 ml/5 ml) and sodium chlorite (298 mg) was added drop by drop to the reaction solution. After the reaction solution had been stirred at room temperature for 2.5 hours, water was added to the reaction solution and the obtained mixture was extracted with dichloromethane. The organic layer was washed with a saturated saline and then dried over magnesium sulfate. The solvent was distilled away under reduced pressure to obtain the title compound (312 mg).

¹H-NMR (400 MHz, CDCl₃) δ (ppm): 1.60 (dd, J=8.0, 4.8 Hz, 1H), 1.71 (dd, J=6.0 Hz, J=4.8 Hz, 1H), 2.14 (dd, J=8.0, 6.0 Hz, 1H), 3.79 (s, 3H), 3.80 (s, 3H), 4.24 (t, J=9.6 Hz, 1H), 4.38 (d, J=9.6 Hz, 1H), 6.30 (dd, J=8.4, 2.8 Hz, 1H), 6.42 (d, J=2.8 Hz, 1H), 6.42 (d, J=8.4 Hz, 1H), 7.24-7.27 (m, 1H), 7.31-7.34 (m, 2H), 7.43-7.45 (m, 2H).

### Production Example 2

### Synthesis of 5-hydroxy-1-ethylpyridin-2(1H)-one (Prep 2)

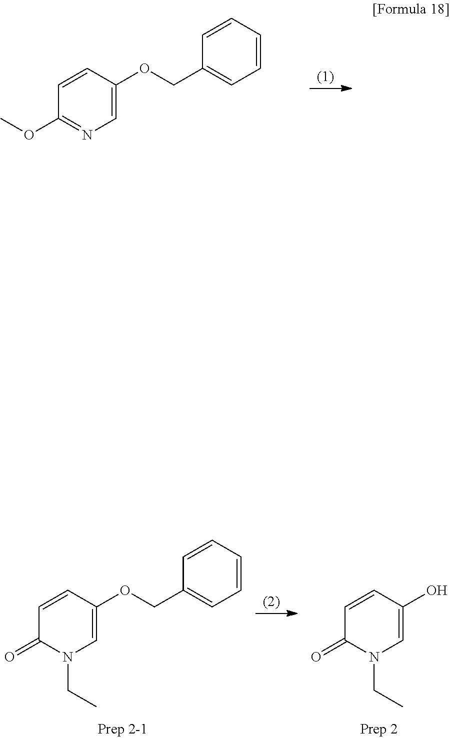

### (1) 5-Benzyloxy-1-ethylpyridin-2(1H)-one (Prep 2-1)

5-Benzyloxy-2-methoxypyridine (CAS No. 1083329-15-0: 4.81 g) was dissolved in acetonitrile (10 ml) and iodoethane (17.8 ml) was added. After the reaction solution had been stirred at 80° C. for 28 hours, the reaction solution was concentrated under reduced pressure. The residue was purified by silica gel column chromatography (n-heptane: ethyl acetate) to obtain the title compound (2.89 g).

¹H-NMR (400 MHz, CDCl₃) δ (ppm): 1.31 (t, J=7.2 Hz, 1H), 3.93 (q, J=7.23 Hz, 1H), 4.89 (s, 2H), 6.54 (d, J=10.0 Hz, 1H), 6.83 (d, J=2.8 Hz, 1H), 7.23 (dd, J=10.0, 2.8 Hz, 1H), 7.33-7.43 (m, 5H).

### (2) 5-Hydroxy-1-ethylpyridin-2(1H)-one (Prep 2)

To compound Prep 2-1 (500 mg), concentrated hydrochloric acid (5 ml) was added and the obtained mixture was stirred at 100° C. for 30 minutes. The reaction solution was concentrated under reduced pressure. To the residue, methanol-toluene was added and concentrated under reduced pressure. This operation was repeated three times. To the obtained solid, ethyl acetate-methanol was added and the reaction solution was filtered and washed with ethyl acetate. The obtained substance was dried in air overnight to obtain the title compound (300 mg).

¹H-NMR. (400 MHz, DMSO-d6) δ (ppm): 1.19 (t, J=7.2 Hz, 1H), 3.87 (q, J=7.2 Hz, 1H), 6.42 (dd, J=9.6, 0.8 Hz, 1H), 7.23 (dd, J=3.2, 0.8 Hz, 1H), 7.26 (dd, J=9.6, 3.2 Hz, 1H).

### Production Example 3

### Synthesis of 1-ethyl-3-methyl-1H-pyrazol-5-ol (Prep 3)

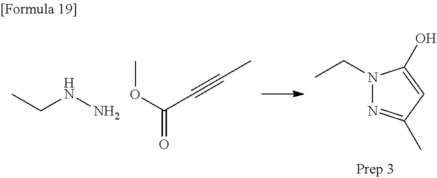

To a dichloromethane solution (15 ml) of ethyl hydrazine (2.0 g), ethyl 2-butynoate (3.88 ml) was added dropwise at 0° C. and the obtained mixture was stirred at the same temperature overnight. Thereafter, the reaction solution was concentrated under reduced pressure and recrystallized with dichloromethane-hexane to obtain the title compound (3.4 g). ¹H-NMR (400 MHz, CDCl₃) δ (ppm): 1.26 (t, J=7.2 Hz, 3H), 2.10 (s, 3H), 3.68 (q, J=7.2 Hz, 2H).

The carboxylic acids of Production Examples 4 and 5 were synthesized by the same method as in Production Example 1.

TABLE 1

| Production | | |
|---|---|---|
| example | Structural formula | NMR and/or MS |
| 4 | 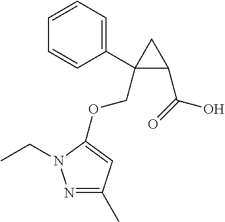 | MS [M + H]⁺ = 301 |
| 5 | 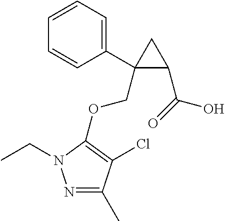 | MS [M + H]⁺ = 335 |

### Production Example 6

### Synthesis of (1S,2R)-2-tert-butyldiphenylsilyloxymethyl)-1-phenylcyclopropyl methanol (Prep 6)

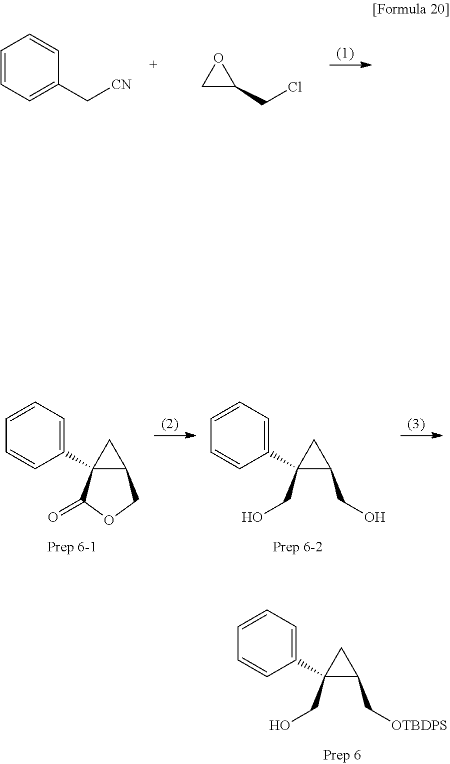

(1) (1S,5R)-1-Phenyl-3-oxabicyclo[3.1.0]hexan-2-one (Prep 6-1)

Phenyl acetonitrile (20 g) was dissolved in THF (500 ml) and NaHMDS (323 ml, 1.06 M) was added dropwise thereto under cooling in an ice-salt bath. The reaction solution was stirred for 2 hours in the same temperature and R-(−)-epichlorohydrin (15.8 g) was added dropwise thereto for 3 hours at 0° C. After stirred for 2 hours at 0° C. of the internal temperature, the reaction solution was stirred at room temperature overnight. The reaction solution was cooled on ice, added a small amount of water dropwise thereto and then concentrated under reduced pressure. To the residue, ethanol (200 ml) and a 1 N potassium hydroxide aqueous solution (200 ml) were added and heated to reflux for 8 hours. After cooling the reaction solution to room temperature, the pH of reaction solution was adjusted to pH<2 with concentrated hydrochloric acid. The obtained solution was stirred at 0° C. for 2 hours and then stirred at room temperature for 1 hour. After the reaction solution was concentrated under reduced pressure, ethyl acetate and water were added to carry out liquid separation. The organic layer was successively washed with a saturated sodium bicarbonate aqueous solution and a saturated saline. The organic layer was dried over magnesium sulfate and concentrated under reduced pressure. The residue was purified by silica gel column chromatography (n-heptane:ethyl acetate) to obtain the title compound (24.7 g).

¹H-NMR (400 MHz, CDCl₃) δ (ppm): 1.37 (t, 1H, J=4.8 Hz), 1.65 (dd, J=7.8, 4.4, 1H), 2.54-2.58 (m, 1H), 4.30 (d, J=9.2, 1H), 4.47 (dd, J=9.4, 4.4 Hz, 1H), 7.25-7.45 (m, 5H).

### (2) (1S,2R)-1-Phenylcyclopropane-1,2-dimethanol (Prep 6-2)

To a THF-methanol solution (200 ml-100 ml) of compound Prep 6-1 (24.7 g), sodium borohydride (10.7 g) was added at 0° C. and the obtained mixture was stirred at room temperature for 1 hour. Under cooling on ice, water was added to the reaction solution, which was concentrated under reduced pressure and extracted with ethyl acetate. The organic layer was washed with a saturated saline, then dried over magnesium sulfate and concentrated under reduced pressure. The residue was purified by silica gel column chromatography (n-heptane:ethyl acetate) to obtain the title compound (20.5 g).

¹H-NMR (400 MHz, CDCl₃) δ (ppm): 0.78 (t, J=5.2 Hz, 1H), 1.87 (dd, J=8.6, 5.2 Hz, 1H), 1.60-1.76 (m, 1H), 3.42 (t, J=11.6 Hz, 1H), 3.57 (dd, J=9.4, 4.4 Hz, 1H), 4.14-4.28 (m, 2H) 7.22-7.44 (m, 5H).

### (3) (1S,2R)-2-(tert-Butyldiphenylsilyloxymethyl)-1-phenylcyclopropyl methanol (Prep 6)

Compound Prep 6-2 (10 g) and imidazole (4.01 g) were dissolved in DMF (90 ml), cooled to −15° C. and then a DMF solution (20 ml) of tert-butyldiphenylsilyl chloride was added dropwise thereto. After the reaction solution had been stirred for 1 hour, methanol was added to the reaction solution and the obtained mixture was stirred at room temperature for 30 minutes. To the organic layer, water was added and the obtained mixture was extracted with ethyl acetate. The organic layer was successively washed with a saturated ammonium chloride aqueous solution, water and a saturated saline and dried over anhydrous magnesium sulfate. The solvent was distilled away under reduced pressure and the residue was purified by silica gel column chromatography (n-heptane: ethyl acetate) to obtain the title compound (10.5 g).

¹H-NMR (400 MHz, CDCl₃) δ (ppm): 0.71 (t, J=5.6 Hz, 1H), 1.04 (dd, J=9.6, 5.2 Hz, 1H), 1.5-1.58 (m, 1H), 3.50 (dd, J=12.4, 1.6 Hz, 1H), 3.53 (dd, J=11.6 Hz, 1H), 3.71 (dd, J=12.4, 1.6 Hz, 1H), 4.10 (t, J=12.0 Hz, 1H), 4.20 (dd, J=12.0, 5.6 Hz, 1H), 7.21-7.46 (m, 10H), 7.7-7.76 (m, 5H)

### Production Example 7

### Synthesis of (1S,2R)-2-[(1-ethyl-3-methyl-1H-pyrazol-4-yl)oxymethyl]-2-phenylcyclopropanecarboxylic acid

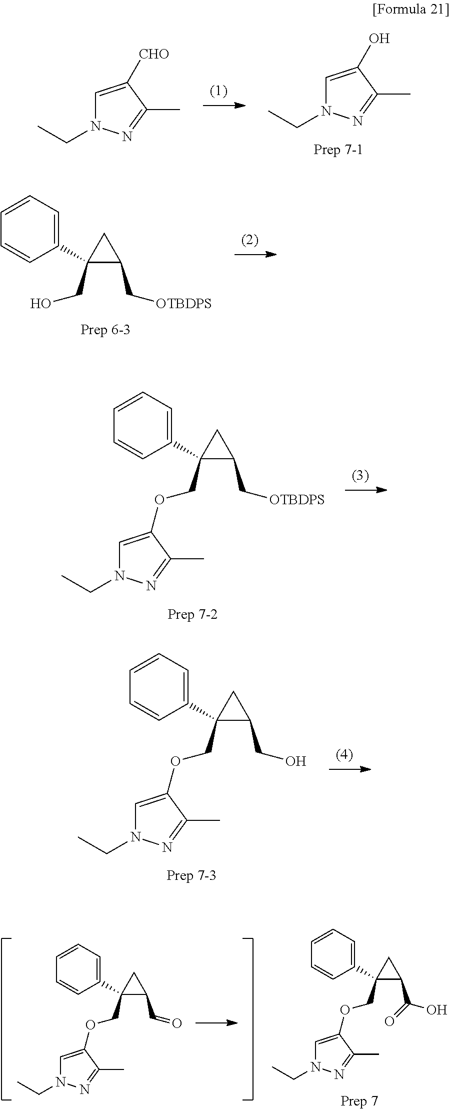

### (1) 1-Ethyl-3-methyl-1H-pyrazol-4-ol (Prep 7-1)

1-Ethyl-3-methyl-1H-pyrazole-4-carbaldehyde (1 g) was dissolved in chloroform (15 ml). To this, m-chloroperbenzoic acid (2.03 g) was added and the obtained mixture was stirred at room temperature for 17 hours. The reaction solution was purified by NH-silica gel column chromatography (n-heptane: ethyl acetate) to obtain the title compound (911 mg).

¹H-NMR (400 MHz, CDCl₃) δ (ppm): 1.38 (t, J=7.2 Hz, 1H), 2.16 (s, 3H), 3.97 (q, J=7.2 Hz, 1H), 6.24 (brs, 1H), 6.99 (s, 1H).

### (2) 4-[(1S,2R)-2-tert-Butyldiphenylsilyloxymethyl-1-phenylcyclopropyl)methoxy]-1-ethyl-3-methyl-1H-pyrazole (Prep 7-2)

To a THF solution (15 ml) of compound Prep 7-1 (545 mg), triphenylphosphine (1.13 g) and compound Prep 6-3 (1.5 g) diisopropylazodicarboxylate (970 μl) was added dropwise at 0° C. and the obtained mixture was stirred at 0° C. for 20 minutes and then stirred at room temperature for 42 hours. The reaction solution was concentrated under reduced pressure and the residue was purified by silica gel column chromatography (n-heptane: ethyl acetate) to obtain the title compound (1.20 g).

¹H-NMR (400 MHz, CDCl₃) δ (ppm): 0.92 (dd, J=6.0, 5.2 Hz, 1H), 1.08 (s, 9H), 1.18 (dd, J=8.8, 5.2 Hz, 1H), 1.35 (t, J=7.2 Hz, 3H), 1.54-1.62 (m, 1H), 2.03 (s, 3H), 3.86 (dd, J=11.2, 8.0 Hz, 1H), 3.94 (q, J=7.2 Hz, 2H), 3.99 (dd, J=11.2, 6.4 Hz, 1H), 3.99 (d, J=10.0 Hz, 1H), 4.02 (d, J=10.0 Hz, 1H), 6.72 (s, 1H), 7.19-7.24 (m, 1H), 7.27-7.45 (m, 10H), 7.66-7.72 (m, 4H).

### (3) {(1S,2R)-2-[(1-Ethyl-3-methyl-1H-pyrazol-4-yl)oxymethyl]-2-phenylcyclopropyl}methanol (Prep 7-3)

To a THF solution (3 ml) of compound Prep 7-2 (1.20 g), tetrabutylammonium fluoride (1M THF solution: 3.42 ml) was added, and the obtained mixture was stirred at room temperature for 25 hours. The reaction solution was concentrated under reduced pressure and the residue was purified by silica gel column chromatography (n-heptane: ethyl acetate) to obtain the title compound (560 mg).

¹H-NMR (400 MHz, CDCl₃) δ (ppm): 0.87 (t, J=5.2 Hz, 1H), 1.22 (dd, J=8.8, 5.2 Hz, 1H), 1.35 (t, J=7.2 Hz, 3H), 1.76-1.84 (m, 1H), 2.10 (s, 3H), 2.89-2.98 (m, 1H), 3.46 (dd, J=11.2, 10.8 Hz, 1H), 3.86 (d, J=10.0 Hz, 1H), 3.95 (q, J=7.2 Hz, 2H), 4.07-4.16 (m, 1H), 4.31 (d, J=10.0 Hz, 1H), 6.89 (s, 1H), 7.21-7.26 (m, 1H), 7.29-7.34 (m, 2H), 7.39-7.44 (m, 2H).

### (4) (1S,2R)-2-[(1-Ethyl-3-methyl-1H-pyrazol-4-yl)oxymethyl]-2-phenylcyclopropanecarboxylic acid (Prep 7)

A dichloromethane solution (5 ml) of oxalyl chloride (425 μl) was cooled to −78° C. To this, a dichloromethane solution (1 ml) of DMSO (705 μl) was added dropwise. Five minutes later, a dichloromethane solution (6 ml) of compound Prep 7-3 (710 mg) was added dropwise to the reaction solution at −78° C. and the obtained mixture was stirred at the same temperature as described above for 60 minutes. To the reaction solution, triethylamine (2.77 ml) was added and the obtained mixture was stirred for 15 minutes and the temperature of the solution was risen to room temperature. After the reaction solution had been stirred at room temperature for 1 hour, an ammonium chloride aqueous solution was added to the reaction solution and the obtained mixture was extracted with ethyl acetate. The organic layer was successively washed with water and a saturated saline and dried over anhydrous magnesium sulfate. After filtration, the filtrate was concentrated under reduced pressure to obtain the corresponding aldehyde.

To an acetone (16 ml)-water (8 ml) solution of the obtained aldehyde, 2-methyl-2-butene (2.63 ml), sodium dihydrogen phosphate (297 mg) and sodium chlorite (1.34 g) were added, and the obtained mixture was stirred at room temperature for 1 hour. To the reaction solution, an ammonium chloride aqueous solution was added and the obtained mixture was extracted with ethyl acetate. The organic layer was successively washed with water and a saturated saline and dried over anhydrous magnesium sulfate. After filtration, the filtrate was concentrated under reduced pressure. The residue was purified by silica gel column chromatography (n-heptane: ethyl acetate). The obtained solid was washed with n-heptane to obtain the title compound (565 mg).

¹H-NMR (400 MHz, CDCl₃) δ (ppm): 1.34 (t, J=7.2 Hz, 3H), 1.51 (dd, J=8.0, 4.8 Hz, 1H), 1.67 (dd, J=6.0, 4.8 Hz, 1H), 1.98 (s, 3H), 2.16 (dd, J=8.0, 6.0 Hz, 1H), 3.89-4.02 (m, 2H), 4.11 (d, J=10.0 Hz, 1H), 4.30 (d, J=10.0 Hz, 1H), 6.86 (s, 1H), 7.23-7.29 (m, 1H), 7.30-7.36 (m, 2H), 7.43-7.48 (m, 2H).

The carboxylic acids of Production Examples 8 to 15 were each synthesized by the same method as in Production Example 7.

TABLE 2

| Production | | |
|---|---|---|
| example | Structural formula | NMR and/or MS |
| 8 | 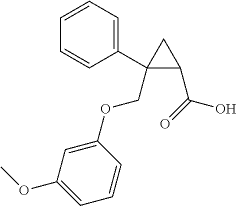 | ¹H-NMR (400 MHz, CDCl₃) δ (ppm): 1.60 (dd, J = 8.0, 5.2 Hz, 1H), 1.72 (dd, J = 6.0, 5.2 Hz, 1H), 2.15 (dd, J = 8.0, 6.0 Hz, 1H), 3.74 (s, 3H), 4.27 (d, J = 10.0 Hz, 1H), 4.40 (d, J = 10.0 Hz, 1H), 6.37-6.48 (m, 2H), 7.10 (t, J = 8.0 Hz, 1H), 7.22-7.28 (m, 1H), 7.29-7.35 (m, 2H), 7.42-7.47 (m, 2H). |
| 9 | 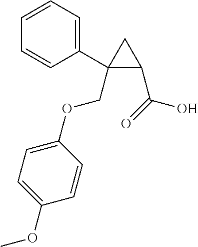 | ¹H-NMR (400 MHz, CDCl₃) δ (ppm): 1.58 (dd, J = 8.4, 4.8 Hz, 1H), 1.70 (dd, J = 6.0, 4.8 Hz, 1H), 2.13 (dd, J = 8.4, 6.0 Hz, 1H), 3.72 (s, 3H), 4.24 (d, J = 10.0 Hz, 1H), 4.36 (d, J = 10.0 Hz, 1H), 6.75 (s, 4H), 7.23-7.27 (m, 1H), 7.29-7.35 (m, 2H), 7.43-7.46 (m, 2H). |
| 10 | 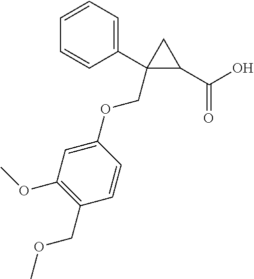 | ¹H-NMR (400 MHz, CDCl₃) δ (ppm): 1.59 (dd, J = 8.0, 4.8 Hz, 1H), 1.72 (dd, J = 6.0, 4.8 Hz, 1H), 2.15 (dd, J = 8.0, 5.6 Hz, 1H), 3.33 (s, 3H), 3.77 (s, 3H), 4.29 (d, J = 10.0 Hz, 1H), 4.38 (s, 2H), 4.43 (d, J = 10.4 Hz, 1H), 6.35-6.39 (m, 2H), 7.12-7.16 (m, 1H), 7.23-7.28 (m, 1H), 7.30-7.35 (m, 2H), 7.42-7.46 (m, 2H). |
| 11 | 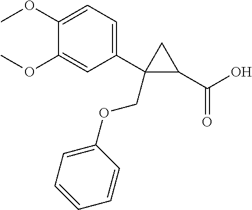 | ¹H-NMR (400 MHz, CDCl₃) δ (ppm): 1.58 (dd, J = 8.4, 5.6 Hz, 1H), 1.69 (t, J = 5.6 Hz, 1H), 2.11 (dd, J = 8.4, 5.6 Hz, 1H), 3.86 (s, 3H), 3.89 (s, 3H), 4.27 (d, J = 9.6 Hz, 1H), 4.37 (d, J = 9.6 Hz, 1H), 6.77-6.84 (m, 3H), 6.87- 6.92 (m, 1H), 6.97 (dd, J = 8.0, 2.0 Hz, 1H), 7.02 (d, J = 2.0 Hz, 1H), 7.18-7.24 (m, 3H). |
| 12 | 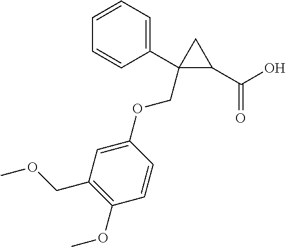 | ¹H-NMR (400 MHz, CDCl₃) δ (ppm): 1.55 (dd, J = 8.0, 5.2 Hz, 1H), 1.69 (t, J = 5.2 Hz, 1H), 2.11-2.17 (m, 1H), 3.39 (s, 3H), 3.75 (s, 3H), 4.25 (d, J = 10.0 Hz, 1H), 4.39 (d, J = 10.0 Hz, 1H), 4.42 (s, 2H), 6.67-6.73 (m, 2H), 6.88- 6.90 (m, 1H), 7.22-7.27 (m, 1H), 7.29-7.34 (m, 2H), 7.42- 7.46 (m, 2H). |
| 13 | 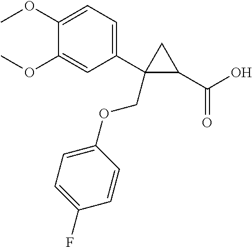 | ¹H-NMR (400 MHz, CDCl₃) δ (ppm): 1.58 (dd, J = 8.0, 4.8 Hz, 1H), 1.67 (dd, J = 6.0, 4.8 Hz 1H), 2.13 (dd, J = 8.0, 6.0 Hz, 1H), 3.86 (s, 3H), 3.89 (s, 3H), 4.24 (d, J = 9.6 Hz, 1H), 4.34 (d, J = 9.6 Hz, 1H), 6.71-6.77 (m, 2H), 6.81 (d, J = 8.4 Hz, 1H), 6.86-6.93 (m, 2H), 6.97 (dd, J = 8.4, 2.0 Hz, 1H), 7.00 (d, J = 2.0 Hz, 1H). |
| 14 | 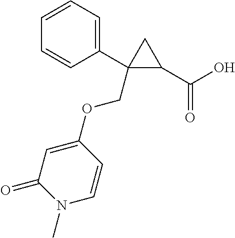 | ¹H-NMR (400 MHz, CDCl₃) δ (ppm): 0.94 (dd, J = 6.0, 5.6 Hz, 1H), 1.26 (dd, J = 8.8, 5.6 Hz, 1H), 1.74-1.83 (m, 1H), 2.16-2.22 (m, 1H), 3.44 (s, 3H), 3.45-3.53 (m, 1H), 3.93 (d, J = 10.4 Hz, 1H), 3.99-4.07 (m, 1H), 4.46 (d, J = 10.4 Hz, 1H), 5.84-5.88 (m, 2H), 7.09-7.13 (m, 1H), 7.21-7.41 (m, 5H). |
| 15 | 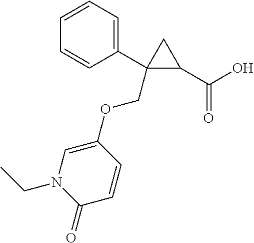 | ¹H-NMR (400 MHz, CDCl₃) δ (ppm): 0.90 (dd, J = 5.6, 5.2 Hz, 1H), 1.23 (dd, J = 8.8, 5.2 Hz, 1H), 1.29 (t, J = 7.2 Hz, 3H), 1.76-1.85 (m, 1H), 2.52-2.58 (m, 1H), 3.46-3.74 (m, 1H), 3.83 (q, J = 7.2 Hz, 2H), 3.89 (d, J = 10.4 Hz, 1H), 4.07-4.15 (m, 1H), 4.30 (d, J = 10.4 Hz, 1H), 6.48 (d, = 9.6 Hz, 1H), 6.73 (d, J = 2.8 Hz, 1H), 7.05 (dd, J = 9.6, 2.8 Hz, 1H), 7.23-7.29 (m, 1H), 7.31-7.36 (m, 2H), 7.38-7.42 (m, 2H). |

### Production Example 16

### Synthesis of (1R,2S)-2-[(1-ethyl-3-methyl-1H-pyrazol-4-yl)oxymethyl]-2-(3-fluorophenyl)cyclopropanecarboxylic acid (Prep 16)

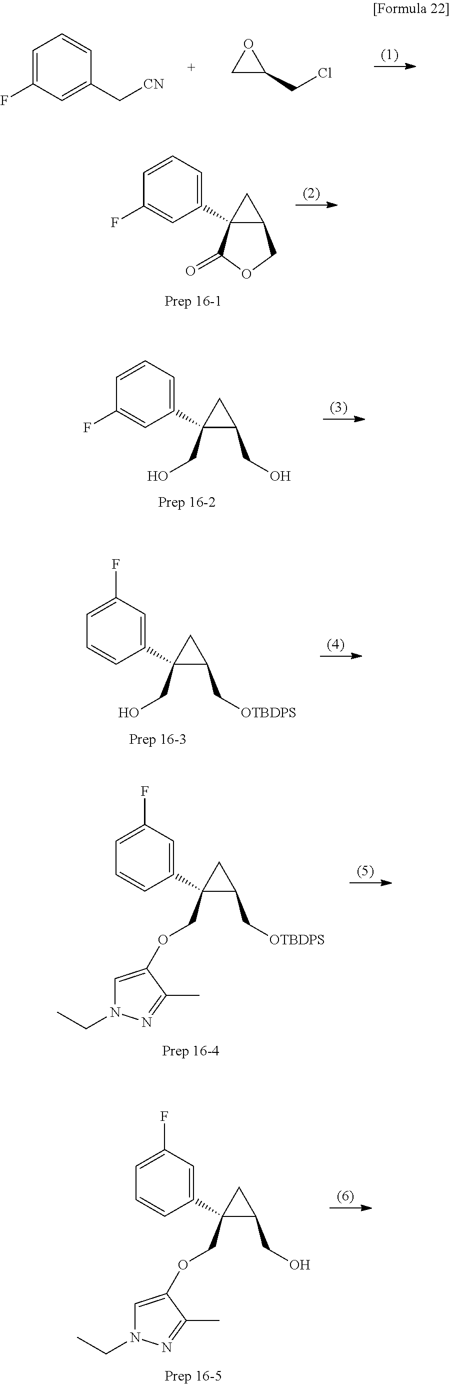

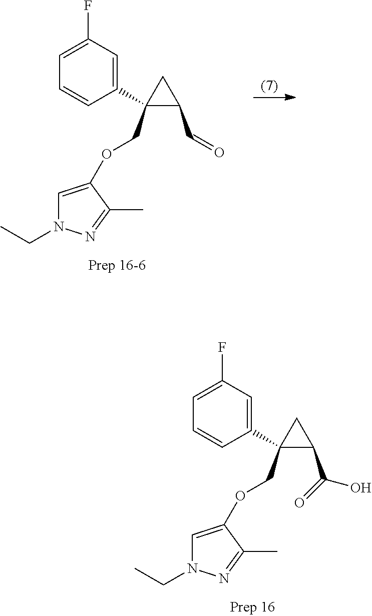

### (1) (1S,5R)-1-(3-Fluorophenyl)-3-oxabicyclo[3.1.0]hexan-2-one (Prep 16-1)

3-Fluorophenylacetonitrile (70 g) was dissolved in THF (500 ml), NaHMDS (1000 ml, 1.06 M) was added dropwise under cooling in an ice-salt bath. After the reaction solution had been stirred in the same condition for 1 hour, R-(−)-epichlorohydrin (40.6 ml) was added dropwise thereto with kept at below 10° C. of the internal temperature. The obtained solution was stirred for 2 hours at around 0° C. of the internal temperature and then stirred at room temperature for 14 hours. The reaction solution was cooled on ice and a small amount of water was added dropwise. The reaction solution was concentrated under reduced pressure and ethanol (700 ml), a 1 N potassium hydroxide aqueous solution (1000 ml) were added to the residue and heated to reflux for 5 hours. After the temperature of the reaction solution was returned to room temperature, a 5 N hydrochloric acid (400 ml) was added and the obtained mixture was stirred at 60° C. for 1 hour. The reaction solution was concentrated under reduced pressure and ethyl acetate and water were added to carry out liquid separation. The organic layer was successively washed with a saturated sodium bicarbonate aqueous solution and a saturated saline and dried over magnesium sulfate. The solvent was concentrated under reduced pressure and the residue was purified by silica gel column chromatography (n-heptane:ethyl acetate) to obtain the title compound (84.9 g).

¹H-NMR (400 MHz, CDCl₃) δ (ppm): 1.41 (t, J=5.2 Hz, 1H), 1.64 (dd, J=8.0, 5.2 Hz, 1H), 2.56-2.63 (m, 1H), 4.30 (d, J=9.2 Hz, 1H), 4.47 (dd, J=9.2, 4.8 Hz, 1H), 6.96-7.02 (m, 1H), 7.16-7.21 (m, 2H), 7.28-7.35 (m, 1H).

### (2) (1S,2R)-1-(3-Fluorophenyl)cyclopropane-1,2-dimethanol (Prep 16-2)

To a THF-methanol solution (440 ml-220 ml) of compound Prep 16-1 (72.7 g), sodium borohydride (25 g) was added at 0° C. and the obtained mixture was stirred at room temperature for 65 hours. Under cooling on ice, water and 5 N hydrochloric acid were added to the reaction solution, which was extracted with ethyl acetate. The organic layer was washed with a saturated saline and then dried over magnesium sulfate. The solvent was concentrated under reduced pressure and the residue was purified by silica gel column chromatography (n-heptane:ethyl acetate) to obtain the title compound (72.7 g).

¹H-NMR (400 MHz, CDCl₃) δ (ppm): 0.80 (t, J=5.0 Hz, 1H), 1.10 (dd, J=8.6, 5.0 Hz, 1H), 1.62-1.71 (m, 1H), 3.41 (t, J=11.4 Hz, 1H), 3.58 (d, J=12.0 Hz, 1H), 4.12-4.25 (m, 2H), 6.90-6.96 (m, 1H), 7.08-7.14 (m, 1H), 7.16-7.21 (m, 1H), 7.24-7.32 (m, 1H).

### (3) {(1S,2R)-[2-(tert-Butyldiphenylsilyloxymethyl)-1-(3-fluorophenyl)cyclopropyl]}methanol (Prep 16-3)

Compound Prep 16-2 (42.4 g) and triethylamine (33.0 ml) were dissolved in dichloromethane (216 ml) and cooled to −20° C. and then tert-butyldiphenylsilyl chloride (56.3 ml) was added dropwise (for approximately 30 minutes; insoluble matter was precipitated at almost the same time as completion of dropping). After the reaction solution had been stirred at −20° C. for 1 hour, stirring was further performed at room temperature for 20 hours. To the reaction solution, water was added and the obtained mixture was extracted with dichloromethane. The organic layer was washed with water and dried over anhydrous magnesium sulfate. The solvent was distilled away under reduced pressure and the residue was purified by silica gel column chromatography (n-heptane: ethyl acetate) to obtain the title compound (67.8 g).

¹H-NMR (400 MHz, CDCl₃) δ (ppm): 0.73 (t, J=5.2 Hz, 1H), 1.04 (dd, J=8.4, 5.2 Hz, 1H), 1.09 (s, 9H), 1.48-1.53 (m, 1H), 3.52 (t, J=12.0 Hz, 1H), 3.56 (dd, J=9.6, 1.6 Hz, 1H), 3.70 (dd, J=9.6, 1.6 Hz, 1H), 4.18 (t, J=12.0 Hz, 1H), 4.20 (dd, J=12.0, 5.2 Hz, 1H), 6.93 (tdd, J=8.0, 2.4, 1.2 Hz, 1H), 7.11 (dt, J=9.6, 2.4 Hz, 1H), 7.20 (dt, J=8.0, 1.2 Hz, 1H), 7.28 (td, J=8.0, 6.0 Hz, 1H), 7.37-7.49 (m, 6H), 7.69-7.74 (m, 4H).

### (4) 4-{[(1S,2R)-2-(tert-Butyldiphenylsilyloxymethyl)-1-(3-fluorophenyl)cyclopropyl]methoxy}-1-ethyl-3-methyl-1H-pyrazole (Prep 16-4)

To a THF solution (25 ml) of compound Prep 16-3 (2.50 g), triphenylphosphine (1.96 g) and compound Prep 7-1 (943 mg), diisopropyl azodicarboxylate (1.67 ml) was added dropwise at 0° C. and the obtained mixture was stirred at room temperature for 20 hours. The reaction solution was concentrated under reduced pressure and the residue was purified by silica gel column chromatography (n-heptane: ethyl acetate) to obtain the title compound (1.94 g).

¹H-NMR (400 MHz, CDCl₃) δ (ppm): 0.94 (dd, J=6.4, 5.2 Hz, 1H), 1.08 (s, 9H), 1.17 (dd, J=8.8, 5.2 Hz, 1H), 1.34 (t, J=7.2 Hz, 3H), 1.54-1.60 (m, 1H), 2.04 (s, 3H), 3.83 (dd, J=11.4, 7.8 Hz, 1H), 3.92-4.00 (m, 5H), 6.76 (s, 1H), 6.90 (ddt, J=7.6, 2.8, 1.2 Hz, 1H), 7.09 (ddd, J=10.4, 2.8, 1.2 Hz, 1H), 7.14-7.17 (m, 1H), 7.22-7.27 (m, 1H), 7.34-7.45 (m, 6H), 7.66-7.69 (m, 4H).

MS [M+H]⁺=543

### (5) {(1R,2S)-2-[(1-Ethyl-3-methyl-1H-pyrazol-4-yl)oxymethyl]-2-(3-fluorophenyl)cyclopropyl}methanol (Prep 16-5)

To a THF solution (4 ml) of compound Prep 16-4 (1.94 g), tetrabutylammonium fluoride (1 M THF solution: 4.3 ml) was added dropwise at room temperature and the obtained mixture was stirred at room temperature for 18 hours. The reaction solution was concentrated under reduced pressure and the residue was purified by silica gel column chromatography (n-heptane:ethyl acetate) to obtain the title compound (0.94 g).

¹H-NMR (400 MHz, CDCl₃) δ (ppm): 0.89 (t, J=5.6 Hz, 1H), 1.23 (dd, J=8.8, 5.6 Hz, 1H), 1.38 (t, J=7.2 Hz, 3H), 1.74-1.82 (m, 1H), 2.11 (s, 3H), 2.87 (dd, J=11.2, 2.0 Hz, 1H), 3.45 (ddd, J=12.8, 10.6, 2.0 Hz, 1H), 3.85 (d, J=10.4 Hz, 1H), 3.97 (q, J=7.2 Hz, 2H), 4.07-4.14 (m, 1H), 4.30 (d, J=10.4 Hz, 1H), 6.93-6.95 (m, 2H), 7.09-7.30 (m, 3H).

MS [M+H]⁺=305

### (6) (1R,2 S)-2-[(1-Ethyl-3-methyl-1H-pyrazol-4-yl)oxymethyl]-2-(3-fluorophenyl)cyclopropanecarbaldehyde (Prep 16-6)

A dichloromethane solution (8 ml) of oxalyl chloride (525 μl) was cooled to −78° C. To this, a dichloromethane solution (2 ml) of DMSO (869 μl) was added dropwise. Ten minutes later, a dichloromethane solution (5 ml) of compound Prep 16-5 (930 mg) was added dropwise to the reaction solution at −78° C. and the obtained mixture was stirred at the same temperature as described above for 60 minutes. To the reaction solution, triethylamine (3.4 ml) was added and the temperature of the reaction solution was increased to room temperature and the reaction solution was stirred for 50 minutes. To the reaction solution, water was added and the obtained mixture was extracted with ethyl acetate. The organic layer was washed with water and a saturated saline solution and then dried over anhydrous magnesium sulfate and filtered. The filtrate was concentrated under reduced pressure and the residue was purified by silica gel column chromatography (n-heptane: ethyl acetate→ethyl acetate) to obtain the title compound (842 mg).

¹H-NMR (400 MHz, CDCl₃) δ (ppm): 1.37 (t, J=7.2 Hz, 3H), 1.62 (dd, J=8.0, 5.2 Hz, 1H), 1.92 (dd, J=6.2, 5.2 Hz, 1H), 2.08 (s, 3H), 2.39 (ddd, J=8.0, 6.2, 3.6 Hz, 1H), 3.93-3.98 (m, 3H), 4.23 (d, J=10.4 Hz, 1H), 6.89 (s, 1H), 6.98 (ddt, J=8.0, 2.8, 1.2 Hz, 1H), 7.16 (ddd, J=10.0, 2.8, 1.2 Hz, 1H), 7.20 (dt, J=8.0, 1.2 Hz, 1H), 7.26-7.33 (m, 1H), 9.77 (d, J=3.6 Hz, 1H).

### (7) (1R,2S)-2-[(1-Ethyl-3-methyl-1H-pyrazol-4-yl)oxymethyl]-2-(3-fluorophenyl)cyclopropanecarboxylic acid (Prep 16)

To an acetone-water solution (10 ml) of compound Prep 16-6 (840 mg), 2-methyl-2-butene (2.95 ml), sodium dihydrogen phosphate (334 mg) and sodium chlorite (629 mg) were added at room temperature and the obtained mixture was stirred for 16 hours. The reaction solution was concentrated under reduced pressure and the residue was purified by silica gel column chromatography (n-heptane: ethyl acetate→ethyl acetate→chloroform:methanol) to obtain the title compound (739 mg).

¹H-NMR (400 MHz, CDCl₃) δ (ppm): 1.35 (t, J=7.2 Hz, 3H), 1.51 (dd, J=8.0, 4.8 Hz, 1H), 1.66 (dd, J=6.0, 4.8 Hz, 1H), 2.04 (s, 3H), 2.16 (dd, J=8.0, 6.0 Hz, 1H), 3.90-4.00 (m, 2H), 4.12 (d, J=10.0 Hz, 1H), 4.25 (d, J=10.0 Hz, 1H), 6.88 (s, 1H), 6.97 (ddt, J=8.0, 2.8, 1.2 Hz, 1H), 7.18 (ddd, J=10.0, 2.8, 1.2 Hz, 1H), 7.22 (dt, J=8.0, 1.2 Hz, 1H), 7.29 (dt, J=8.0, 6.4 Hz, 1H).

MS [M+H]⁺=319

### Production Example 17

### Synthesis of 1-ethyl-3,5-dimethyl-1H-pyrazol-4-ol (Prep 17)

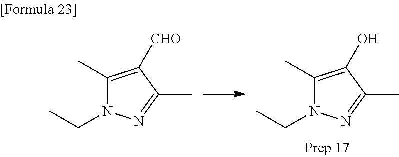

1-Ethyl-3,5-dimethyl-1H-pyrazole-4-carbaldehyde (CAS No. 701911-46-8; 1.55 g) was dissolved in chloroform (20 ml) and m-chloroperbenzoic acid (2.29 g) was added and the obtained mixture was stirred at room temperature for 91 hours. To the reaction solution, ethyl acetate was added and filtered with NH silica gel. The filtrate was concentrated under reduced pressure and the residue was purified by silica gel column chromatography (n-heptane: ethyl acetate) to obtain the title compound (883 mg).

¹H-NMR (400 MHz, CDCl₃) δ (ppm): 1.30 (t, J=7.2 Hz, 3H), 2.21 (s, 3H), 2.15 (s, 3H), 3.93 (q, J=7.2 Hz, 2H), 5.48 (s, 1H).

### Production Example 18

### Synthesis of 1-methyl-1,2-dihydropyridazine-3,6-dione (Prep 18)

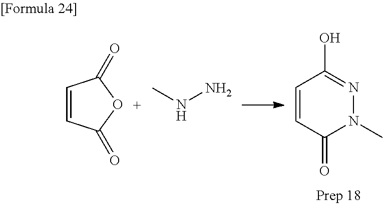

Maleic anhydride (3.0 g) was dissolved in acetic acid (30 ml) and cooled to 0° C. To the acetic acid solution, methyl hydrazine (1.62 ml) was added at 0° C. and the reaction solution was warmed up to room temperature and stirred for 22 hours. A precipitated white solid was collected by filtration and washed with ethanol to obtain the title compound (2.28 g).

¹H-NMR (400 MHz, CDCl₃) δ (ppm): 3.44 (s, 3H), 6.86 (d, J=10.0 Hz, 1H), 7.03 (d, J=10.0 Hz, 1H), 11.04 (brs, 1H).

The carboxylic acids of Production Examples 19 to 22 were each synthesized by the same manner as in Production Example 7.

TABLE 3-1

| Production | | |
|---|---|---|
| example | Structural formula | NMR and/or MS |
| Prep19 | 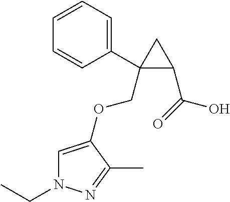 | ¹H-NMR (400 MHz, CDCl₃) δ (ppm): 1.34 (t, J = 7.2 Hz, 3H), 1.51 (dd, J = 8.0, 4.8 Hz, 1H), 1.67 (dd, t, J = 6.0, 4.8 Hz, 1H), 1.98 (s, 3H), 2.16 (dd, J = 8.0, 6.0 Hz, 1H), 3.89- 4.02 (m, 2H), 4.11 (d, J = 10.0 Hz, 1H), 4.30 (d, J = 10.0 Hz, 1H), 6.86 (s, 1H), 7.23-7.29 (m, 1H), 7.30-7.36 (m, 2H), 7.43-7.48 (m, 2H). |
| Prep20 | 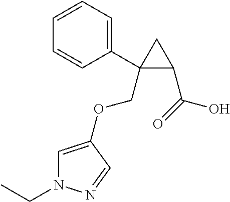 | ¹H-NMR (400 MHz, CDCl₃) δ (ppm): 1.38 (t, J = 7.2 Hz, 3H), 1.52 (dd, J = 8.0, 5.2 Hz, 1H), 1.68 (dd, J = 6.0, 5.2 Hz, 1H), 2.16 (dd, J = 8.0, 6.0 Hz, 1H), 4.01 (q, J = 7.2 Hz, 2H), 4.20 (d, J = 10.0 Hz, 1H), 4.34 (d, J = 10.0 Hz, 1H), 6.98 (d, J = 0.8 Hz, 1H), 7.15 (d, J = 0.8 Hz, 1H), 7.23-7.28 (m, 1H), 7.30-7.35 (m, 2H), 7.41-7.45 (m, 2H). |
| Prep21 | 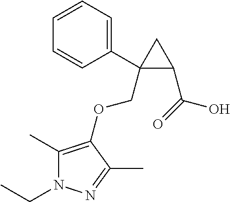 | ¹H-NMR (400 MHz, CDCl₃) δ (ppm): 1.25 (t, J = 7.2 Hz, 3H), 1.46 (dd, J = 8.0, 4.8 Hz, 1H), 1.63 (dd, J = 6.0, 4.8 Hz, 1H), 1.86 (s, 3H), 1.91 (s, 3H), 2.21 (dd, J = 8.0, 6.0 Hz, 1H), 3.87 (q, J = 7.2 Hz, 2H), 4.13 (d, J = 10.0 Hz, 1H), 4.24 (d, J = 10.0 Hz, 1H), 7.25-7.30 (m, 1H), 7.32-7.37 (m, 2H), 7.47-7.51 (m, 2H) |
| Prep22 | 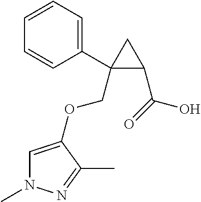 | ¹H-NMR (400 MHz, CDCl₃) δ (ppm): 1.51 (dd, J = 8.0, 5.2 Hz, 1H), 1.66 (dd, J = 6.0, 5.2 Hz, 1H), 1.98 (s, 3H), 2.17 (dd, J = 8.0, 6.0 Hz, 1H), 3.69 (s, 3H), 4.10 (d, J = 10.0 Hz, 1H), 4.29 (d, J = 10.0 Hz, 1H), 6.83 (s, 1H), 7.24- 7.29 (m, 1H), 7.31-7.36 (m, 2H), 7.44-7.47 (m, 2H). |

The carboxylic acids of Production Examples 23 to 28 were each also synthesized according to Production Example 7.

TABLE 4

| Production | |
|---|---|
| example | Structural formula, MS |
| Prep23 | 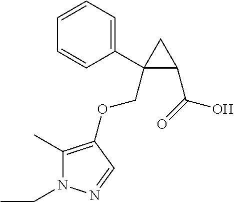 MS [M + H]⁺ = 301 |
| Prep24 | 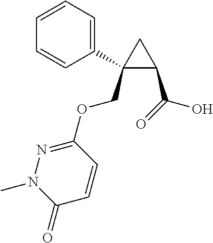 MS [M + H]⁺ = 301 |
| Prep25 | 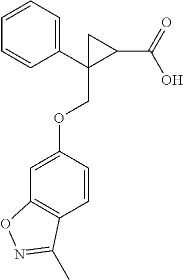 MS [M + H]⁺ = 324 |
| Prep26 | 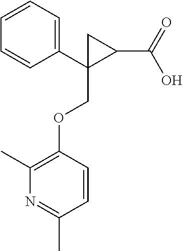 MS [M + H]⁺ = 298 |
| Prep27 | 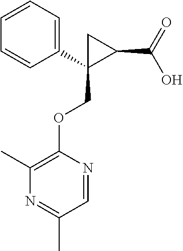 MS [M + H]⁺ = 299 |
| Prep28 | 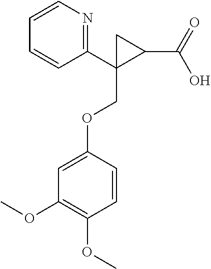 MS [M + H]⁺ = 330 |

### Production Example 29

### Synthesis of (1R,2S)-2-(1,3-dimethyl-6-oxo-1,6-dihydropyridazin-4-yl oxymethyl)-2-phenylcyclopropanecarboxylic acid (Prep 29)

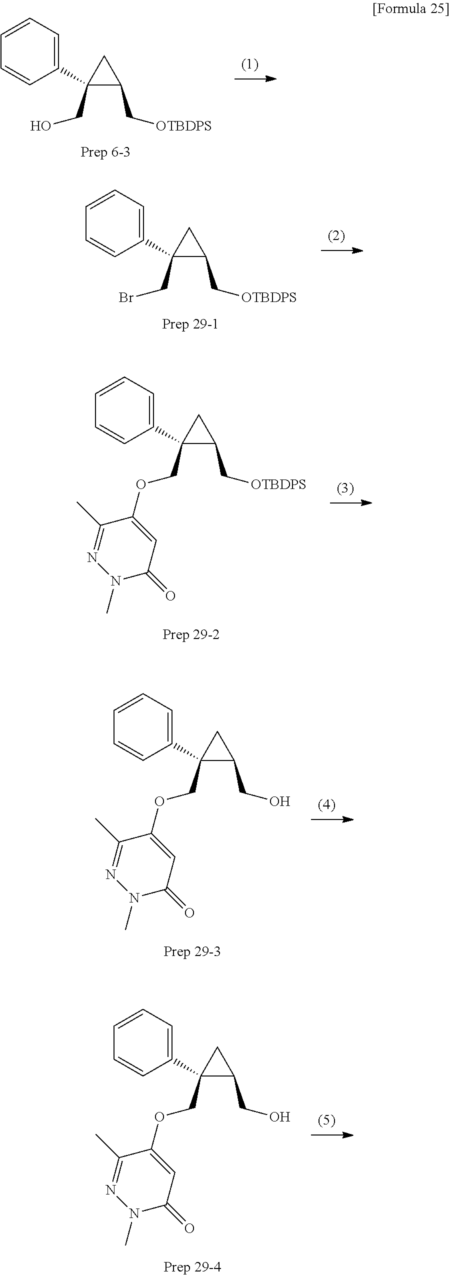

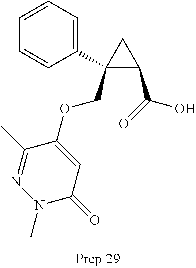

### (1) tert-Butyl-{[(1R,2S)-2-bromomethyl-2-phenylcyclopropyl]methoxy}diphenylsilane (Prep 29-1)

Compound Prep 6-3 (2.00 g) and carbon tetrabromide (2.39 g) were dissolved in dichloromethane (30 ml) and the reaction solution was cooled to 0° C. and then triphenylphosphine (1.89 g) was added and the obtained mixture was stirred for 1.5 hours. To the reaction solution, water was added and the obtained mixture was extracted with ethyl acetate. The organic layer was successively washed with water and a saturated saline and dried over anhydrous sodium sulfate. The solvent was distilled away under reduced pressure and the residue was purified by silica gel column chromatography (n-heptane: ethyl acetate) to obtain the title compound (2.26 g).

MS [M+Na]⁺=503

### (2) 5-{[(1S,2R)-2-(tert-Butyldiphenylsilyloxymethyl)-1-phenylcyclopropyl]methoxy}-2,6-dimethyl-2H-pyridazin-3-one (Prep 29-2)

Compound Prep 29-1 (1 g) and 5-hydroxy-2,6-dimethyl-2H-pyridazin-3-one (322 mg) were dissolved in DMF (10 ml). To this, potassium carbonate (577 mg) was added and the obtained mixture was stirred at 80° C. for 1.5 hours. To the reaction solution, water was added and the obtained mixture was extracted with ethyl acetate. The organic layer was successively washed with water and a saturated saline and dried over anhydrous magnesium sulfate. The solvent was distilled away under reduced pressure and the residue was purified by silica gel column chromatography (n-heptane: ethyl acetate) to obtain the title compound (221 mg).

¹H-NMR (400 MHz, CDCl₃) δ (ppm): 0.92 (t, J=5.2 Hz, 1H), 1.08 (s, 9H), 1.21 (dd, J=8.8, 5.2 Hz, 1H), 1.61-1.66 (m, 1H), 2.09 (s, 3H), 3.65 (s, 3H), 3.70-3.74 (m, 1H), 4.00-4.05 (m, 2H), 4.10 (d, J=10.0 Hz, 1H), 5.91 (s, 1H), 7.22-7.44 (m, 11H), 7.66 (d, J=7.2 Hz, 4H).

### (3) 5-{[(1S,2R)-2-Hydroxymethyl-1-phenylcyclopropyl]methoxy}-2,6-dimethyl-2H-pyridazin-3-one (Prep 29-3)

To a THF solution (2.2 ml) of compound Prep 29-2 (221 mg), tetrabutylammonium fluoride (1 M THF solution: 0.82 ml) was added dropwise at 0° C. and the obtained mixture was stirred at room temperature for 2 hours. To the reaction solution, water was added and the obtained mixture was extracted with ethyl acetate. The organic layer was successively washed with water and a saturated saline and dried over anhydrous magnesium sulfate. The solvent was distilled away under reduced pressure and the residue was purified by silica gel column chromatography (n-heptane: ethyl acetate→ethyl acetate:methanol) to obtain the title compound (117 mg).

¹H-NMR (400 MHz, CDCl₃) δ (ppm): 0.97 (t, J=5.6 Hz, 1H), 1.26-1.30 (m, 1H), 1.74-1.80 (m, 1H), 2.17 (brs, 1H), 2.21 (s, 3H), 3.60 (dd, J=12.0, 9.2 Hz, 1H), 3.64 (s, 3H), 4.00-4.05 (m, 2H), 4.31 (d, J=10.4 Hz, 1H), 6.06 (s, 1H), 7.25 (tt, J=7.6, 1.6 Hz, 1H), 7.32 (tt, J=7.6, 1.6 Hz, 2H), 7.39 (tt, J=7.6, 1.6 Hz, 2H).

### (4) (1R,2S)-2-(1,3-Dimethyl-6-oxo-1,6-dihydropyridazin-4-yl oxymethyl)-2-phenylcyclopropanecarbaldehyde (Prep 29-4)

A dichloromethane solution (10 ml) of oxalyl chloride (65.8 μl) was cooled to −78° C. and a dichloromethane solution (5 ml) of DMSO (111 μl) was added dropwise and then stirred for 10 minutes. To the reaction solution, a dichloromethane solution (15 ml) of compound Prep 29-3 (117 mg) was added dropwise. After the reaction solution had been stirred at −78° C. for 30 minutes, triethylamine (434 μl) was added and further stirred at room temperature for 1 hour. To the reaction solution, water was added and the obtained mixture was extracted with dichloromethane. The extract was dried over anhydrous sodium sulfate. The solvent was distilled away under reduced pressure and the residue was purified by silica gel column chromatography (n-heptane: ethyl acetate) to obtain the title compound (84.8 mg).

MS [M+H]⁺=299

### (5) (1R,2S)-2-(1,3-Dimethyl-6-oxo-1,6-dihydropyridazin-4-yl oxymethyl)-2-phenylcyclopropanecarboxylic acid (Prep 29)

Compound Prep 29-4 (84.8 mg), 2-methyl-2-butene (147 μl) and sodium dihydrogen phosphate (68.1 mg) were dissolved in a mixed solvent of acetone and water (5 ml/1 ml) and sodium chlorite (51.4 mg) was added. After the reaction solution had been stirred at room temperature for 2 hours, the reaction solution was concentrated under reduced pressure. The residue was purified by silica gel column chromatography (n-heptane: ethyl acetate ethyl acetate:methanol) to obtain the title compound (76.0 mg).

¹H-NMR (400 MHz, CDCl₃) δ (ppm): 1.52 (dd, J=8.0, 5.6 Hz, 1H), 1.78 (t, J=5.6 Hz, 1H), 2.18 (s, 3H), 2.29 (dd, J=8.0, 6.0 Hz, 1H), 3.61 (s, 3H), 4.26 (d, J=10.0 Hz, 1H), 4.38 (d, J=10.0 Hz, 1H), 6.54 (s, 1H), 7.28 (t, J=6.8 Hz, 1H), 7.34 (t, J=6.8 Hz, 2H), 7.45 (d, J=6.8 Hz, 2H).

### Production Example 30

### Synthesis of (1R,2S)-2-(1-ethyl-3-methyl-6-oxo-1,6-dihydropyridazin-4-yl oxymethyl)-2-phenylcyclopropanecarboxylic acid (Prep 30)

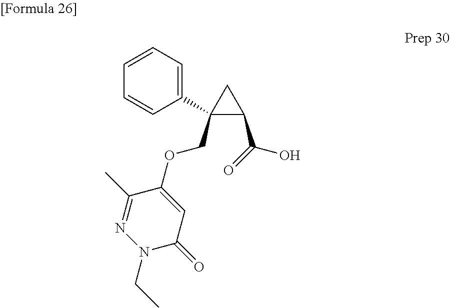

The compound was synthesized according to the process of Production Example 29.

MS [M+H]⁺=329

### Production Example 31

### Synthesis of 2-[2-(1-ethyl-1H-pyrazol-4-yl)ethyl]-2-phenylcyclopropanecarboxylic acid (Prep 31)

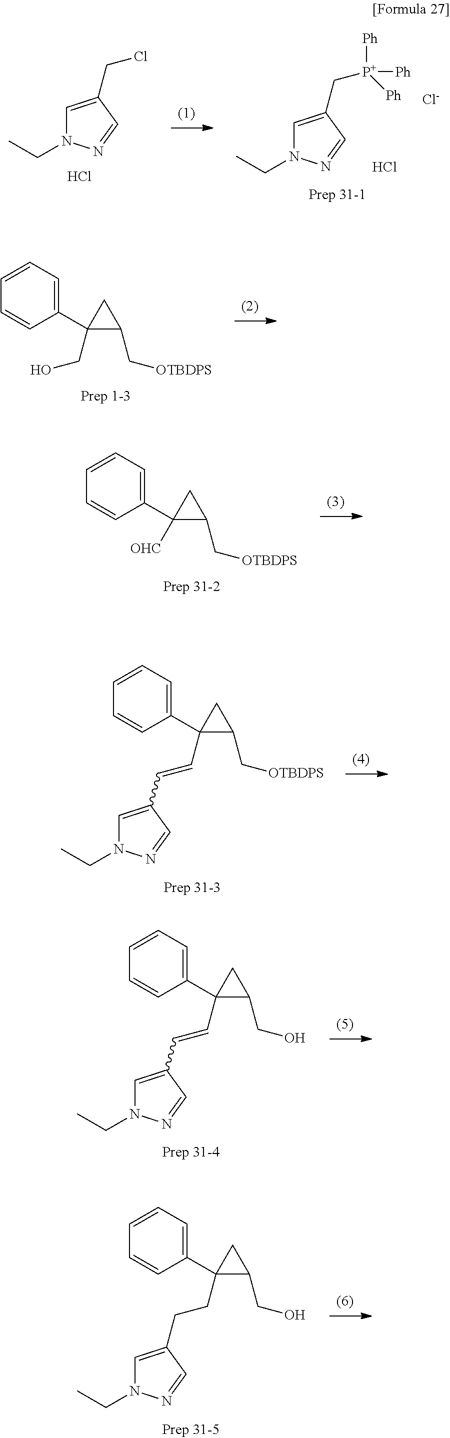

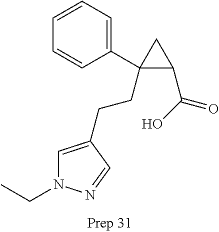

### (1) [(1-Ethyl-1H-pyrazol-4-yl)methyl]triphenylphosphonium chloride hydrochloride (Prep 31-1)

4-(Chloromethyl)-1-ethyl-1H-pyrazole hydrochloride (2.32 g) was dissolved in acetonitrile (25 ml). To this, triphenylphosphine (3.36 g) was added and the obtained mixture was stirred at 80° C. for 20 hours. The reaction solution was concentrated under reduced pressure to obtain the title compound (5.73 g).

¹H-NMR (400 MHz, DMSO-d6) δ (ppm): 1.22 (t, J=7.2 Hz, 3H), 4.01 (q, J=7.2 Hz, 2H), 5.00 (d, J=14.0 Hz, 2H), 6.93 (d, J=2.0 Hz, 1H), 7.24 (d, J=2.0 Hz, 1H), 7.66-7.79 (m, 12H), 7.88-7.94 (m, 3H).

### (2) 2-(tert-Butyldiphenylsilyloxymethyl)-1-phenylcyclopropanecarbaldehyde (Prep 31-2)

A dichloromethane solution (44 ml) of oxalyl chloride (2.06 ml) was cooled to −78° C. To this, a dichloromethane solution (6 ml) of DMSO (3.41 ml) was added dropwise. Five minutes later, a dichloromethane solution (10 ml) of compound Prep 1-3 was added dropwise to the reaction solution at −78° C. and the obtained mixture was stirred at the same temperature as described above for 30 minutes. To the reaction solution, triethylamine (13.4 ml) was added and the obtained mixture was stirred for 15 minutes. Thereafter, the temperature of the reaction solution was increased to room temperature. After the reaction solution had been stirred at room temperature for 1 hour, water was added to the reaction solution and the obtained mixture was extracted with ethyl acetate. The organic layer was successively washed with an ammonium chloride aqueous solution, water and a saturated saline and dried over anhydrous magnesium sulfate. After filtration, the filtrate was concentrated under reduced pressure and the residue was purified by silica gel column chromatography (n-heptane: ethyl acetate) to obtain the title compound (4.65 g).

¹H-NMR (400 MHz, CDCl₃) δ (ppm): 1.07 (s, 9H), 1.52 (dd, J=8.8, 4.8 Hz, 1H), 1.75 (dd, J=7.6, 4.8 Hz, 1H), 1.99-2.07 (m, 1H), 3.69 (dd, J=10.2, 9.6 Hz, 1H), 4.07 (dd, J=10.2, 5.2 Hz, 1H), 7.30-7.46 (m, 4H), 7.63-7.72 (m, 11H), 9.66 (s, 1H).

### (3) 4-{(E,Z)-2-[2-(tert-Butyldiphenylsilyloxymethyl)-1-phenylcyclopropyl]vinyl}-1-ethyl-1H-pyrazole (Prep 31-3)

Compound Prep 31-1 (4.82 g) was suspended in THF (30 ml) and the obtained mixture was stirred under cooling on ice. To this, potassium tert-butoxide (2.43 g) was added. To this, DMSO (30 ml) was added and the obtained mixture was stirred at room temperature for 15 minutes. To this, a THF solution (30 ml) of compound Prep 31-2 (3.00 g) was added and the obtained mixture was stirred at room temperature for 3 hours. To the reaction solution, water was added and the obtained mixture was extracted with ethyl acetate. The organic layer was successively washed with water and a saturated saline and dried over anhydrous magnesium sulfate. After filtration, the filtrate was concentrated under reduced pressure. The residue was purified by silica gel column chromatography (n-heptane: ethyl acetate) to obtain the title compound (2.21 g) as an isomeric mixture.

¹H-NMR (400 MHz, CDCl₃) δ (ppm): 0.75-1.80 (m, 15H), 3.58-4.15 (m, 4H), 5.90-6.50 (m, 2H), 7.12-7.72 (m, 15H).

### (4) {2-[(E,Z)-2-(1-ethyl-1H-pyrazol-4-yl)vinyl]-2-phenylcyclopropyl}methanol (Prep 31-4)

To a THF solution (7 ml) of compound Prep 31-3 (2.21 g), tetrabutylammonium fluoride (1 M THF solution: 6.54 ml) was added and the obtained mixture was stirred at room temperature for 28 hours. The reaction solution was concentrated under reduced pressure and the residue was purified by silica gel column chromatography (n-heptane: ethyl acetate) to obtain the title compound (1.01 g) as an isomeric mixture.

¹H-NMR (400 MHz, CDCl₃) δ (ppm): 0.92-1.89 (m, 6H), 3.55-4.15 (m, 4H), 5.83-6.64 (m, 2H), 7.12-7.47 (m, 5H).

### (5) {2-[2-(1-Ethyl-1H-pyrazol-4-yl)ethyl]-2-phenylcyclopropyl}methanol (Prep 31-5)

To an ethanol solution (50 ml) of compound Prep 31-4 (1.01 g), 10% palladium-carbon (water content: 50%) (400 mg) was added and catalytic hydrogen reduction was performed at room temperature and normal atmospheric pressure. The reaction solution was filtered with Celite and the filtrate was concentrated under reduced pressure to obtain the title compound (995 mg).

¹H-NMR (400 MHz, CDCl₃) δ (ppm): 0.57 (dd, J=6.0, 4.8 Hz, 1H), 1.10-1.16 (m, 1H), 1.35-2.04 (m, 3H), 1.43 (t, J=7.2 Hz, 3H), 2.36-2.44 (m, 2H), 3.70-3.79 (m, 1H), 3.84-3.92 (m, 1H), 4.08 (q, J=7.2 Hz, 2H), 7.07-7.35 (m, 7H)

### (6) 2-[2-(1-Ethyl-1H-pyrazol-4-yl)ethyl]-2-phenylcyclopropanecarboxylic acid (Prep 31)

The title compound was obtained from compound Prep 31-5 by the same processes as in Production Examples 1-(7), (8).

¹H-NMR (400 MHz, CDCl₃) δ (ppm): 1.26 (t, J=7.0, 1H), 1.37-2.44 (m, 6H), 1.42 (t, J=7.2 Hz, 3H), 4.04-4.16 (m, 2H), 7.05-7.38 (m, 7H).

### Production Example 32

### Synthesis of 2-[2-(3,4-Dimethoxyphenyl)ethyl]-2-phenylcyclopropanecarbaldehyde (Prep 32)

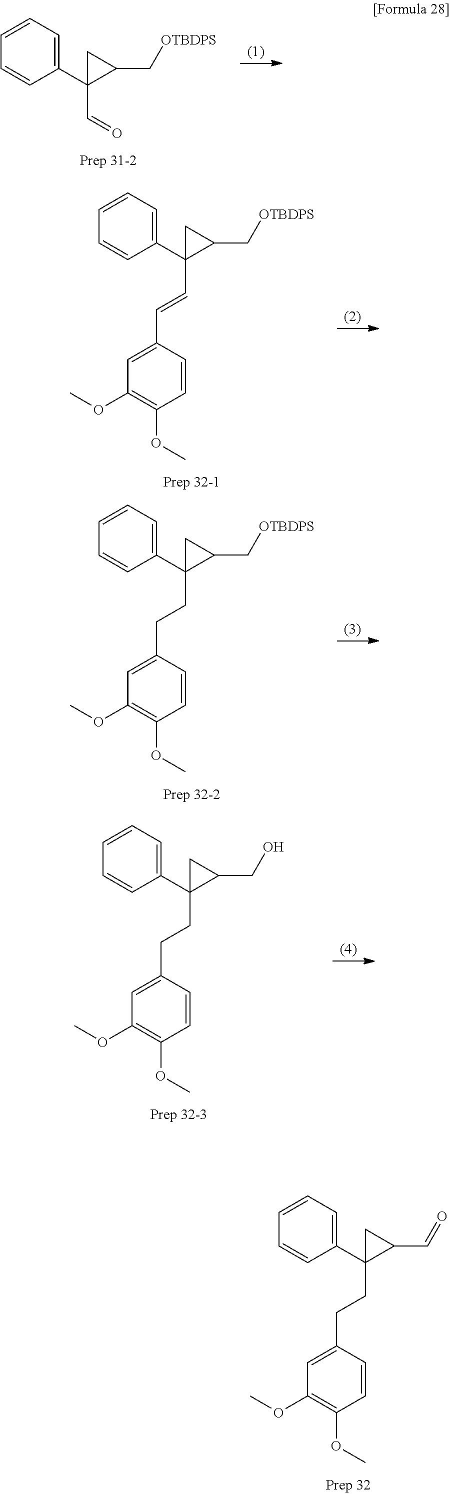

### (1) tert-Butyl{2-[(E)-2-(3,4-dimethoxyphenyl)vinyl]-2-phenylcyclopropyl methoxy}diphenylsilane (Prep 32-1)

To a THF solution (3 ml) of diethyl 3,4-dimethoxybenzylphosphonate (167 mg), sodium hydride (60% oil dispersion: 23.1 mg) was added under cooling on ice and the obtained mixture was stirred for 10 minutes. To this, a THF solution (1 nil) of compound Prep 31-2 (200 mg) was added and the obtained mixture was stirred at room temperature overnight. To the reaction mixture, a saturated ammonium chloride aqueous solution was added, ethyl acetate was added to carry out liquid separation and extraction. The organic layer was dried over magnesium sulfate and the solvent was concentrated under reduced pressure. The obtained residue was purified by NH-silica gel column chromatography (n-heptane: ethyl acetate) to obtain the title compound (45 mg).

MS [M+Na]⁺=571

### (2) tert-Butyl{2-[2-(3,4-dimethoxyphenyl)ethyl]-2-phenylcyclopropyl methoxy}diphenylsilane (Prep 32-2)

To an ethanol solution (5 ml) of compound Prep 32-1 (300 mg), 10% palladium-carbon (water content: 50%) (30 mg) was added. After the reaction solution had stirred for 1 hour under a hydrogen atmosphere, ammonium formate (138 mg) was added and stirred at 60° C. for 10 minutes. The reaction mixture was filtered with Celite and washed with ethyl acetate. The filtrate was concentrated under reduced pressure to obtain a crude title compound (301 mg).

MS [M+Na]⁺=573

### (3) {2-[2-(3,4-Dimethoxyphenyl)ethyl]-2-phenylcyclopropyl}methanol (Prep 32-3)

To a THF solution (2 ml) of compound Prep 32-2 (300 mg), tetrabutylammonium fluoride (1 M THF solution: 1.64 ml) was added dropwise at room temperature and the obtained mixture was stirred at room temperature for 4 hours. The reaction mixture was concentrated under reduced pressure and the obtained residue was purified by silica gel column chromatography (n-heptane: ethyl acetate) to obtain the title compound (79 mg).

MS [M+Na]⁺=335

### (4) 2-[2-(3,4-Dimethoxyphenyl)ethyl]-2-phenylcyclopropanecarbaldehyde (Prep 32)

A dichloromethane solution (2 ml) of oxalyl chloride (43.5 μl) was cooled to −78° C. To this, a dichloromethane solution (500 μl) of DMSO (71.9 μl) was added dropwise. Five minutes later, to the reaction solution, a dichloromethane solution (1.5 ml) of compound Prep 32-3 (79 mg) was added dropwise at −78° C. and the obtained mixture was stirred at the same temperature as described above for 45 minutes. To the reaction mixture, triethylamine (282 μl) was added to remove a coolant and the temperature of the reaction mixture was increased. To the reaction mixture, a saturated ammonium chloride solution was added and extract was performed with dichloromethane. The organic layer was dried over anhydrous magnesium sulfate and filtered. The filtrate was concentrated under reduced pressure and the residue was purified by silica gel column chromatography (n-heptane: ethyl acetate) to obtain the title compound (36.2 mg).

¹H-NMR (400 MHz, CDCl₃) δ (ppm): 1.54-1.72 (m, 2H), 2.08-2.58 (m, 5H), 6.54-6.60 (m, 2H), 6.74 (d, J=8.0 Hz, 1H), 7.26-7.39 (m, 5H), 7.65 (d, J=4.8 Hz, 1H).

### Production Example 33

### Synthesis of {2-[2-(2-ethyl-4-methyl-1,3-thiazol-5-yl)ethyl]-2-phenylcyclopropyl}methanol (Prep 33)

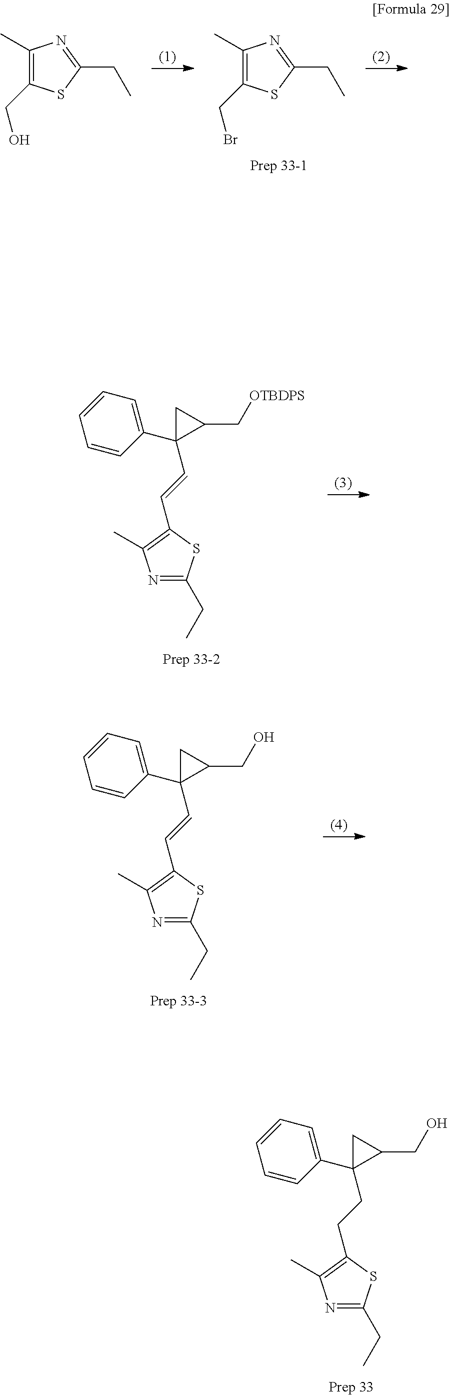

### (1) 5-Bromomethyl-2-ethyl-4-methyl-1,3-thiazole (Prep 33-1)

(2-Ethyl-4-methyl-1,3-thiazol-5-yl)methanol (1 g) was dissolved in chloroform (10 ml) and phosphorus tribromide (658 μl) was added dropwise under cooling on ice and the obtained mixture was stirred for 30 minutes while maintaining the temperature. Thereafter, a saturated sodium bicarbonate aqueous solution was added thereto. The reaction mixture was subjected to liquid separation and extraction with chloroform. The organic layer was dried over magnesium sulfate and the solvent was concentrated under reduced pressure. The obtained residue was purified by silica gel column chromatography (n-heptane: ethyl acetate) to obtain the title compound (278 mg).

¹H-NMR (400 MHz, CDCl₃) δ (ppm): 1.36 (t, J=7.6 Hz, 3H), 2.36 (s, 3H), 2.94 (q, J=7.6 Hz, 2H), 4.65 (s, 2H).

### (2) 5-{(E)-2-[2-(tert-Butyldiphenylsilyloxymethyl)-1-phenylcyclopropyl]vinyl}-2-ethyl-4-methyl-1,3-thiazole (Prep 33-2)

Compound Prep 33-1 (278 mg) was dissolved in toluene (10 ml) and triphenylphosphine was added and the obtained mixture was stirred at 110° C. for 2 days. Crystal grains were collected by filtration from the reaction mixture, dried under reduced pressure to obtain a Wittig salt (541 mg). To a THF solution (10 ml) of the Wittig salt obtained, n-butyl lithium (2.69 M n-hexane solution: 416 μl) was added dropwise under cooling on ice and the obtained mixture was stirred for 15 minutes while maintaining the temperature. To the reaction mixture, the TI-IF solution (4 ml) of Prep 32-2 was added dropwise and the obtained mixture was stirred for 1 hour while maintaining the temperature and then stirred at room temperature for 4 hours. To the reaction mixture, a saturated ammonium chloride aqueous solution was added and subjected to liquid separation and extraction with ethyl acetate. The organic layer was dried over magnesium sulfate and the solvent was concentrated under reduced pressure. The obtained residue was purified by silica gel column chromatography (n-heptane:ethyl acetate) to obtain the title compound (445 mg).

MS [M+H]⁺=538

### (3) {2-[(E)-2-(2-Ethyl-4-methyl-1,3-thiazol-5-yl)vinyl]-2-phenylcyclopropyl}methanol Prep 33-3)

To a THF solution (10.0 ml) of compound Prep 33-2 (445 mg), tetrabutylammonium fluoride (1 M THF solution: 992 μl) was added dropwise at room temperature and the obtained mixture was stirred at room temperature overnight. The reaction solution was concentrated under reduced pressure and the residue was purified by silica gel column chromatography (n-heptane: ethyl acetate) to obtain the title compound (198 mg).

MS [M+H]⁺=300.

### (4) {2-[2-(2-Ethyl-4-methyl-1,3-thiazol-5-yl)ethyl]-2-phenylcyclopropyl}methanol (Prep 33)

To a methanol solution (3.0 ml) of compound Prep 33-3 (198 mg), 10% palladium-carbon (water content: 50%) (80 mg) was added and the obtained mixture was stirred at room temperature for 2 hours under a hydrogen atmosphere. The reaction solution was filtered with Celite, the filtrate obtained was concentrated under reduced pressure. The residue was purified by silica gel column chromatography (n-heptane: ethyl acetate) to obtain the title compound (199 mg).

MS [M+H]⁺=302.

### Production Example 34

### Synthesis of (1R,2S)-2-[2-(3-difluoromethyl-5-methyl-1H-1,2,4-triazol-1-yl)ethyl}-2-phenylcyclopropanecarboxylic acid (Prep 34)

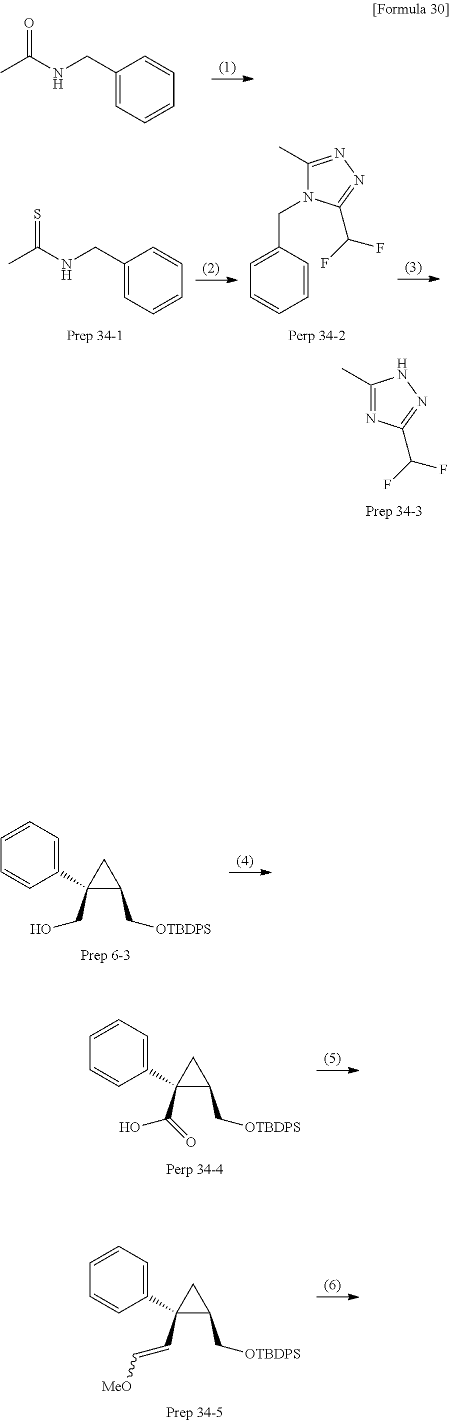

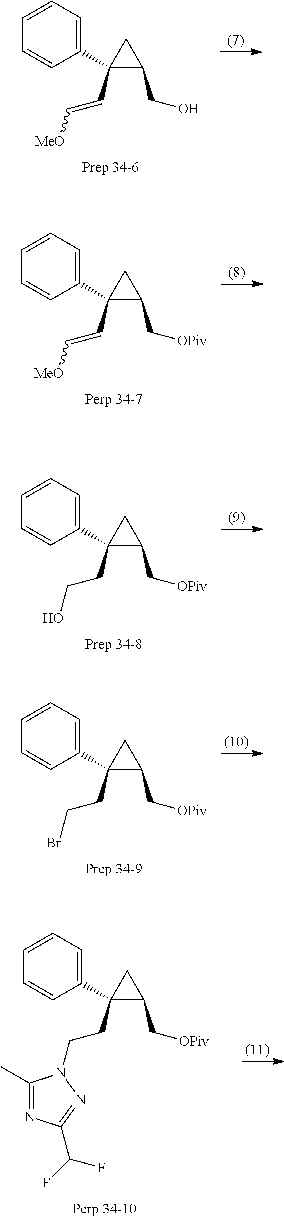

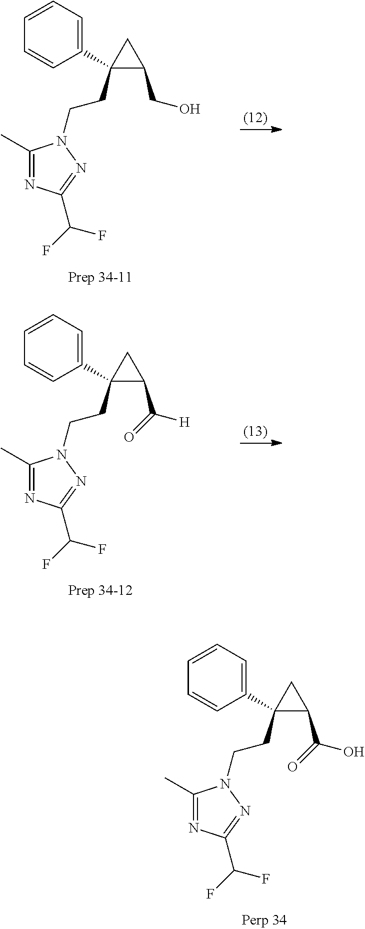

### (1) N-Benzyl-ethanethioamide (Prep 34-1)

To a THF solution (100 ml) of N-benzylacetamide (5.0 g), a Lawesson's reagent (6.77 g) was added and the obtained mixture was stirred at 90° C. for 2 hours. Thereafter, a saturated sodium bicarbonate aqueous solution was added to the reaction solution, diluted with ethyl acetate and washed with water and a saturated saline. The organic layer was dried over anhydrous magnesium sulfate and filtered. The filtrate was concentrated under reduced pressure and the residue was purified by silica gel column chromatography (ethyl acetate:methanol) to obtain the title compound (5.20 g).

¹H-NMR (400 MHz, CDCl₃) δ (ppm): 2.59 (s, 3H), 4.82 (d, J=5.2 Hz, 2H), 7.33-7.38 (m, 5H).

### (2) 4-Benzyl-3-difluoromethyl-5-methyl-4H-1,2,4-triazole (Prep 34-2)

To a THF solution (50 ml) of compound Prep 34-1 (667 mg), 2,2-difluoroacetohydrazide (500 mg) and mercury acetate (966 mg) were added and the obtained mixture was stirred at room temperature overnight. The reaction solution was filtered with Celite and the residue was diluted with ethyl acetate, washed with a saturated sodium bicarbonate aqueous solution and purified by silica gel column chromatography (n-heptane:ethyl acetate) to obtain the title compound (530 mg).

¹H-NMR (400 MHz, CDCl₃) δ (ppm): 2.32 (s, 3H), 4.11 (brs, 2H), 5.99 (t, J=53.6 Hz, 1H), 7.05-7.40 (m, 5H).

### (3) 3-Difluoromethyl-5-methyl-4H-1,2,4-triazole (Prep 34-3)

To an ethanol solution (20 ml) of compound Prep 34-2 (530 mg), 10% palladium-carbon (water content: 50%) (200 mg) and acetic acid (200 μl) were added to perform hydrogenation and the obtained mixture was stirred at room temperature overnight. The reaction solution was filtered with Celite and the filtrate was concentrated under reduced pressure to obtain the title compound (300 mg).

¹H-NMR (400 MHz, CDCl₃) δ (ppm): 2.32 (s, 3H), 5.99 (t, J=53.6 Hz, 1H).

### (4) (1S,2R)-2-(tert-Butyldiphenylsilyloxymethyl)-1-phenylcyclopropanecarbaldehyde (Prep 34-4)

A dichloromethane solution (80 ml) of oxalyl chloride (1.67 ml) was cooled to −78° C. To this, a dichloromethane solution (10 ml) of DMSO (2.73 ml) was added dropwise. Fifteen minutes later, a dichloromethane solution (6 ml) of compound Prep 6-3 (4.0 g) was added dropwise to the reaction solution at −78° C. and the obtained mixture was stirred at the same temperature as described above for 60 minutes. To the reaction solution, triethylamine (10.7 ml) was added. The temperature of the reaction solution was increased to 0° C. and the reaction solution was stirred for 2 hours. The reaction solution was diluted with ethyl acetate and washed with a saturated ammonium chloride aqueous solution, a saturated sodium bicarbonate aqueous solution and a saturated saline. The organic layer was dried over anhydrous magnesium sulfate and filtered. The filtrate was concentrated under reduced pressure and the residue was purified by silica gel column chromatography (n-heptane: ethyl acetate) to obtain the title compound (4.0 g).

¹H-NMR (400 MHz, CDCl₃) δ (ppm): 1.067 (s, 9H), 1.52 (dd, J=8.8, 4.8 Hz, 1H), 1.74 (dd, J=8.8, 4.8 Hz, 1H), 1.98-2.08 (m, 1H), 3.69 (dd, J=11.4, 9.2 Hz, 1H), 4.07 (dd, J=11.6, 9.2 Hz, 1H) 7.25-7.46 (m, 11H), 7.46-7.73 (m, 4H), 9.70 (s, 1H).

### (5) Mixture of tert-butyl({(1R,2R)-2-[(E)-2-methoxyvinyl]-2-phenylcyclopropyl}methoxy)diphenylsilane (Prep 34-5)

To a THF solution (64.9 ml) of (methoxymethyl)triphenylphosphonium chloride (10.0 g), n-butyl lithium (2.6 M n-hexane solution: 10.4 ml) was added dropwise at −78° C. over 30 minutes and the obtained mixture was stirred for 30 minutes in the same condition. While the reaction solution was maintained at −78° C., a THF solution (10 ml) of compound Prep 34-4 (4.0 g) was added dropwise over 30 minutes. Thereafter, the reaction solution was stirred at 0° C. for 3 hours. To the reaction solution, a saturated ammonium chloride aqueous solution was added and the obtained mixture was extracted with ethyl acetate. The organic layer was washed with a sodium bicarbonate aqueous solution and a saturated saline and then dried over magnesium sulfate. The solvent was concentrated under reduced pressure and the residue was purified by silica gel column chromatography (n-heptane: ethyl acetate) to obtain the title compound (3.96 g).

¹H-NMR (400 MHz, CDCl₃) δ (ppm): 0.76-1.32 (m, 10H), 1.46-1.64 (m, 0.5H), 1.96-2.12 (m, 1H), 4.64 (d, J=6.4 Hz, 0.5H) 5.06 (d, J=12.4 Hz, 0.5H), 5.88 (d, J=6.4 Hz, 0.5H), 6.24 (d, J=12.8 Hz, 0.5H) 7.14-7.45 (m, 10H), 7.67-7.73 (m, 5H).

### (6) Mixture of {(1R,2R)-2-[(E)-2-methoxyvinyl]-2-phenylcyclopropyl}methanol (Prep 34-6)

To a THF solution (50 ml) of compound Prep 34-5 (3.96 g), tetrabutylammonium fluoride (1 M THF solution: 26.9 ml) was added dropwise at 0° C. and the obtained mixture was stirred at room temperature overnight. The reaction solution was concentrated under reduced pressure and the residue was purified by silica gel column chromatography (n-heptane: ethyl acetate) to obtain the title compound (1.57 g).

¹H-NMR (400 MHz, CDCl₃) δ (ppm): 0.78 (dd, J=12.0, 4.8 Hz, 0.5H), 0.95 (t, J=5.2 Hz, 0.5H), 1.14-1.19 (m, 1H), 1.59-1.69 (m, 0.5H), 1.73-1.77 (m, 0.5H), 2.60-2.66 (m 0.5H), 3.28-3.38 (m, 1H), 3.52 (s, 1.5H), 3.62 (s, 1H), 3.82-3.98 (m, 1H), 4.65 (d, J=6.4 Hz, 0.5H) 5.03 (d, J=12.4 Hz, 0.5H), 6.05 (d, J=6.4 Hz, 0.5H), 6.21 (d, J=12.4 Hz, 0.5H), 7.13-7.33 (m, 5H).

### (7) Mixture of {(1R,2R)-2-[(E)-2-methoxyvinyl]-2-phenylcyclopropyl)methyl pivalate Prep 34-7)

To a dichloromethane solution of compound Prep 34-6 (1.53 g), pivaloyl chloride (1.38 g) and triethylamine (3.13 ml) were added at 0° C. and the obtained mixture was stirred at room temperature for 7 hours. To the reaction solution, ethyl acetate and a saturated ammonium chloride aqueous solution were added. The organic layer was fractionated and washed with a saturated sodium bicarbonate aqueous solution, water and a saturated saline. The organic layer was concentrated under reduced pressure. The residue was purified by silica gel column chromatography (n-heptane: ethyl acetate) to obtain the title compound (2.64 g).

¹H-NMR (400 MHz, CDCl₃) δ (ppm): 0.88 (d, J=5.2 Hz, 0.5H), 0.98 (t, J=5.2 Hz, 0.5H), 1.13-1.31 (m, 10H), 1.56-1.69 (m, 1H), 3.48 (s, 1.5H), 3.59 (s, 1.5H), 3.93-4.04 (m, 1H), 4.28-4.40 (m, 1H), 4.56 (d, J=6.4 Hz, 0.5H), 5.03 (d, J=12.8 Hz, 0.5H), 6.00 (d, J=6.4 Hz, 0.5H), 6.26 (d, J=12.8 Hz, 0.5H), 7.13-7.32 (m, 5H).

### (8) [(1R,2R-2-2-Hydroxyethyl)-2-phenylcyclopropyl]methyl pivalate (Prep 34-8)

To an acetonitrile-water solution (20 ml) of compound Prep 34-7 (2.64 g), concentrated hydrochloric acid (216 μl) was added dropwise and the obtained mixture was stirred at 50° C. for 1 hour. The reaction solution was extracted with ethyl acetate, concentrated and then dissolved in THF (100 ml). To this, sodium borohydride (732 mg) was added at 0° C. and the obtained mixture was stirred at room temperature for 2 hours. To the reaction solution, ethyl acetate and a saturated ammonium chloride aqueous solution were added. The organic layer was fractionated and washed with a saturated sodium bicarbonate aqueous solution, water and a saturated saline. The organic layer was concentrated under reduced pressure and the residue was purified by silica gel column chromatography (n-heptane: ethyl acetate) to obtain the title compound (1.26 g).

¹H-NMR (400 MHz, CDCl₃) δ (ppm): 0.67 (t, J=4.8 Hz, 1H), 1.17-1.23 (m, 1H), 1.28 (s, 9H), 1.30-1.48 (m, 1H), 1.81-1.88 (m, 1H), 2.00-2.06 (m, 1H), 3.53-3.63 (m, 2H), 3.97 (dd, J=12.0, 4.8 Hz, 1H), 4.52 (dd, J=8.0, 5.2 Hz, 1H), 7.18-7.35 (m, 5H).

### (9) [(1R,2R)-2-(2-Bromoethyl)-2-phenylcyclopropyl]methyl pivalate (Prep 34-9)

To a dichloromethane solution (100 ml) of compound Prep 34-8 (1.26 g), triphenylphosphine (2.4 g) and carbon tetrabromide (4.53 g) were added at 0° C. and the obtained mixture was stirred at room temperature overnight. To the reaction solution, water was added and the obtained mixture was extracted with ethyl acetate. The organic layer was washed with a saturated saline and concentrated under reduced pressure. The residue was purified by silica gel column chromatography (n-heptane: ethyl acetate) to obtain the title compound (1.46 g).

¹H-NMR (400 MHz, CDCl₃) δ (ppm): 0.75 (t, J=5.6 Hz, 1H), 1.23 (dd, J=10.0, 6.0 Hz, 1H), 1.28 (s, 9H), 1.43-1.50 (m, 1H), 2.04-2.12 (m, 1H), 2.29-2.36 (m, 1H), 3.89-3.94 (m, 1H), 4.54 (dd, J=11.8, 5.6 Hz, 1H), 7.21-7.33 (m, 5H).

### (10) {(1R,2S)-2-[2-(3-Difluoromethyl-5-methyl-1H-1,2,4-triazol-1-yl)ethyl]-2-phenylcyclopropyl}methyl pivalate (Prep 34-10)

Compound Prep 34-3 (200 mg) and sodium hydride (60% oil dispersion, 21.2 mg) were dissolved in DMF (10 ml), compound Prep 34-9 (235 mg) was added thereto and the obtained mixture was stirred at 70° C. overnight. To the reaction solution, a saturated saline was added and the obtained mixture was extracted with ethyl acetate. The organic layer was dried over anhydrous magnesium sulfate and the filtrate was concentrated under reduced pressure. The residue was purified by silica gel column chromatography (n-heptane: ethyl acetate) to obtain the title compound (1.40 mg).

¹H-NMR (400 MHz, CDCl₃) δ (ppm): 0.59 (t, J=5.2 Hz, 1H), 1.21-1.27 (m, 1H), 1.26 (s, 9H), 1.43-1.48 (m, 1H), 2.11-2.22 (m, 1H), 2.23 (s, 3H), 2.32-2.35 (m, 1H), 3.88-4.03 (m, 3H), 4.56 (dd, J=12.0, 4.8 Hz, 1H), 4.52 (dd, J=8.0, 5.2 Hz, 1H), 6.59 (t, J=13.6 Hz, 1H), 7.24-7.35 (m, 5H).

### (11) {(1R,2S)-2-[2-(3-Difluoromethyl-5-methyl-1H-1,2,4-triazol-1-yl)ethyl]-2-phenylcyclopropyl}methanol (Prep 34-11)

Compound Prep 34-10 (140 mg) was dissolved in an ethanol-1 N sodium hydroxide aqueous solution (3 ml-6 ml) and the obtained mixture was stirred at 70° C. for 1 hour. The obtained mixture was extracted with ethyl acetate and the organic layer was dried over anhydrous magnesium sulfate and then the filtrate was concentrated under reduced pressure. The residue was purified by silica gel column chromatography (n-heptane: ethyl acetate) to obtain the title compound (80 mg).

¹H-NMR (400 MHz, CDCl₃) δ (ppm): 0.57 (t, J=5.2 Hz, 1H), 1.11 (dd, J=9.0, 5.2 Hz, 1H), 1.48-1.53 (m, 1H), 2.20-2.40 (m, 2H), 2.24 (s, 3H), 3.56-3.58 (m, 1H), 3.94-4.19 (m, 3H), 6.59 (t, J=13.6 Hz, 1H), 7.22-7.34 (m, 5H).

### (12) (1R,2S)-2-[2-(3-Difluoromethyl-5-methyl-1H-1,2,4-triazol-1-yl)ethyl]-2-phenylcyclopropanecarbaldehyde (Prep 34-12)

A dichloromethane solution (5 ml) of oxalyl chloride (62.4 μl) was cooled to −78° C. To this, a dichloromethane solution (2 ml) of DMSO (102 μl) was added dropwise. Thirty minutes later, to the reaction solution, a dichloromethane solution (3 ml) of compound Prep 34-11 (80 mg) was added dropwise at −78° C. and the obtained mixture was stirred at the same temperature as described above for 30 minutes. To the reaction solution, triethylamine (3.53 ml) was added and the temperature of the reaction solution was increased to 0° C. and the obtained mixture was stirred for 30 minutes. To the reaction solution, water and a saturated sodium bicarbonate aqueous solution were added and the obtained mixture was extracted with ethyl acetate. The organic layer was dried over anhydrous magnesium sulfate and filtered. The filtrate was concentrated under reduced pressure and the residue was purified by silica gel column chromatography (n-heptane: ethyl acetate) to obtain the title compound (58 mg).

¹H-NMR (400 MHz, CDCl₃) δ (ppm): 1.55-1.63 (m, 2H), 2.27-2.49 (m, 3H), 2.28 (s, 3H), 3.86-3.99 (m, 2H), 6.60 (t, J=13.6 Hz, 1H), 7.22-7.34 (m, 5H), 9.77 (d, J=3.6H, 1H).

### (13) (1R,2S)-2-[2-(3-Difluoromethyl-5-methyl-1H-1,2,4-triazol-1-yl)ethyl]-2-phenylcyclopropanecarboxylic acid (Prep 34)

To an acetone-water solution (2 ml-1 ml) of compound Prep 34-12 (58 mg), 2-methyl-2-butene (101 μl), anhydrous sodium dihydrogen phosphate (45.6 mg) and sodium chlorite (34.4 mg) were added at room temperature and the obtained mixture was stirred for 2 hours. The reaction solution was extracted with ethyl acetate and washed with a saturated saline. The organic layer was dried over anhydrous magnesium sulfate and then filtered. The filtrate was concentrated under reduced pressure to obtain the title compound (58 mg).

MS [M+H]⁺=322

### Production Example 35

### Synthesis of 3-methoxy-5-methyl-1H-1,2,4-triazole (Prep 35)

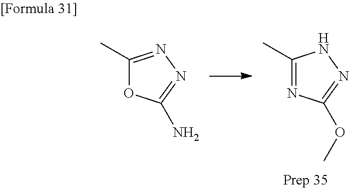

To a methanol solution (20 ml) of 5-methyl-1,3,4-oxazol-2-ylamine (2.0 g), potassium hydroxide (2.27 g) was added and the obtained mixture was stirred at 90° C. overnight. Thereafter, to the reaction solution, water and ammonia water were added. The reaction solution was extracted with ethyl acetate-chloroform and concentrated under reduced pressure to obtain the title compound (1.15 g).

¹H-NMR (400 MHz, CDCl₃) δ (ppm): 2.39 (s, 3H), 4.00 (s, 3H).

### Production Example 36

### Synthesis of 2-[2-(3-methoxy-5-methyl-1H-1,2,4-pyrazol-1-yl)ethyl]-2-phenylcyclopropanecarboncarboxylic acid (Prep 36)

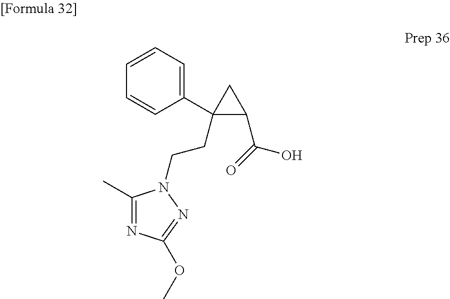

The compound was synthesized using compound Prep 35 according to the method of Production Example 34.

MS [M+H]⁺=302

### Production Example 37

### Synthesis of 4-chloro-2-ethyl-1H-imidazole (Prep 37)

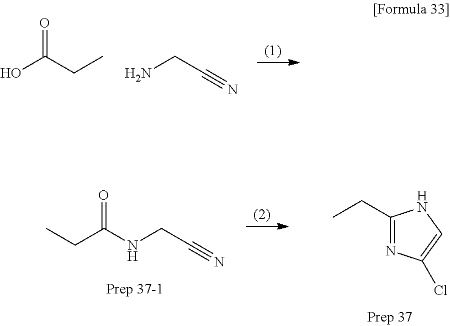

### (1) N-Cyanomethyl propaneamide (Prep 37-1)

To a THF solution (40 ml) of propionic acid (2.0 g), WSCI (7.76 g), 1-hydroxybenzotriazole (5.47 g) and N,N-diisopropylethylamine (14.1 ml) were added and the obtained mixture was stirred at room temperature overnight. Thereafter, water was added to the reaction solution and the obtained mixture was extracted with ethyl acetate. The organic layer obtained was washed with a saturated ammonium chloride aqueous solution, a saturated sodium bicarbonate aqueous solution and a saturated saline and concentrated under reduced pressure. The residue was purified by silica gel column chromatography (ethyl acetate:methanol) to obtain the title compound (2.97 g).

¹H-NMR (400 MHz, CDCl₃) δ (ppm): 1.18 (t, J=7.6 Hz, 3H), 2.29 (q, J=7.6 Hz, 2H), 4.20 (d, J=8.0 Hz 1H).

### (2) 4-Chloro-2-ethyl-1H-imidazole (Prep 37)

To an acetonitrile solution (16.8 ml) of compound Prep 37-1 (1.0 g), triphenylphosphine (5.86 g) and carbon tetrachloride (2.15 ml) were added and the obtained mixture was stirred at 45° C. overnight. Thereafter, the reaction solution was concentrated under reduced pressure and the residue was purified by silica gel column chromatography (ethyl acetate:methanol) to obtain the title compound (214 mg).

¹H-NMR (400 MHz, CDCl₃) δ (ppm): 1.25 (t, J=7.6 Hz, 3H), 2.70 (q, J=7.6 Hz, 2H), 6.78 (s, 1H).

### Production Example 38

### Synthesis of 2-[2-(4-chloro-2-ethyl-1H-imidazol-1-yl)ethyl]-2-phenylcyclopropanecarboxylic acid (Prep 38)

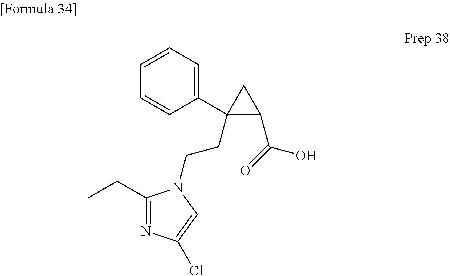

The compound was synthesized using compound Prep 37-2 according to the method of Production Example 34.

MS [M+H]⁺=319

The compounds of Production Examples 39 to 45 were each synthesized according to the process of Production Example 34.

TABLE 5

| Production | |
|---|---|
| example | Structural formula, MS |
| Prep39 | 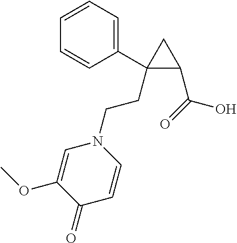 MS [M + H]⁺ = 314 |
| Prep40 | 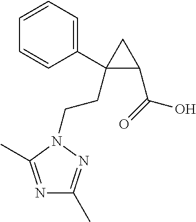 MS [M + H]⁺ = 286 |
| Prep41 | 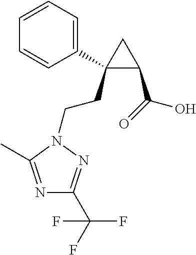 MS [M + H]⁺ = 340 |
| Prep42 | 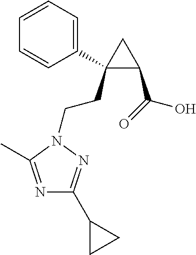 MS [M + H]⁺ = 312 |
| Prep43 | 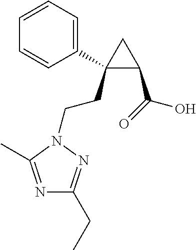 MS [M + H]⁺ = 300 |
| Prep44 | 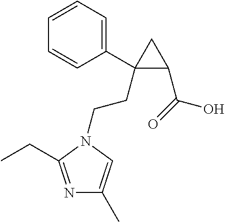 MS [M + H]⁺ = 298 |
| Prep45 | 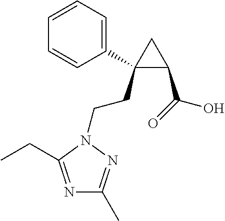 MS [M + H]⁺ = 300 |

### Production Example 46

### Synthesis of 2-[2-(5-methyl-1,3,4-oxadiazol-2-yl)ethyl]-2-phenylcyclopropanecarboxylic acid (Prep 46)

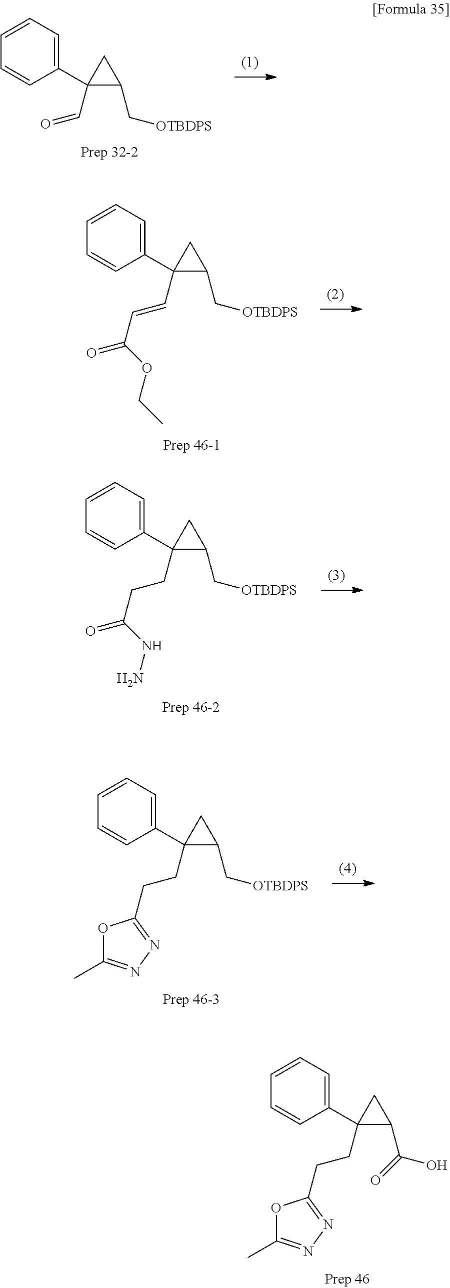

### (1) Ethyl(2E)-3-[(2-tert-butyldiphenylsilyloxymethyl)-1-phenylcyclopropyl]acrylate (Prep 46-1)

A THF solution (10 ml) of triethyl phosphonoacetate (720 μl) was stirred under a nitrogen atmosphere under cooling on ice. To this, n-butyl lithium (2.69 M n-hexane solution, 1.34 ml) was added dropwise and the obtained mixture was stirred under cooling on ice for 30 minutes. Thereafter, a THF solution (10 ml) of compound Prep 31-2 (1 g) was added and the obtained mixture was stirred at room temperature for 22 hours. After a saturated saline was added, the obtained mixture was extracted with ethyl acetate and the organic layer was dried over anhydrous magnesium sulfate. The solvent was distilled away under reduced pressure and the residue was purified by silica gel column chromatography (n-heptane→n-heptane: ethyl acetate) to obtain the title compound (1.03 g).

¹H-NMR (400 MHz, CDCl₃) δ (ppm): 1.07 (s, 9H), 1.12-1.36 (m, 4H), 1.44 (dd, J=8.8, 5.2 Hz, 1H), 1.83-1.94 (m, 1H), 3.70 (dd, J=11.2, 8.8 Hz, 1H), 3.98-4.26 (m, 3H), 5.35 (d, J=15.6 Hz, 1H), 7.05 (d, J=15.6 Hz, 1H), 7.14-7.76 (m, 15H).

### (2) 3-[2-(tert-Butyldiphenylsilyloxymethyl)-1-phenylcyclopropyl]propionic acid hydrazide (Prep 46-2)

To an ethyl acetate solution (25 ml) of compound Prep 46-1 (1 g), 10% palladium-carbon (water content: 50%) (1 g) was added and the obtained mixture was stirred at room temperature for 2 hours and 15 minutes under a hydrogen atmosphere. The reaction solution was filtered with Celite and concentrated under reduced pressure to obtain a reduced form of the compound. To an ethanol solution (10 ml) of the reduced form obtained, hydrazine monohydrate (1.03 ml) was added and the obtained mixture was stirred at room temperature for 17.5 hours, further stirred at 60° C. for 2.5 hours, and further stirred at 90° C. for 5.5 hours. To this, hydrazine monohydrate (3.09 ml) was further added and the obtained mixture was stirred for 39.5 hours. Thereafter, a saturated sodium bicarbonate aqueous solution was added and the obtained mixture was extracted with ethyl acetate. After washed with water and a saturated saline, the organic layer was dried over anhydrous magnesium sulfate. The solvent was distilled away under reduced pressure and the residue was purified by silica gel column chromatography (n-heptane: ethyl acetate) to obtain the title compound (0.792 g).

¹H-NMR (400 MHz, CDCl₃) δ (ppm): 0.40-4.20 (m, 9H), 1.11 (s, 9H), 6.34-6.48 (m, 2H), 7.04-7.76 (m, 15H).

MS [M+H]⁺=473

### (3) 2-{2-[2-(tert-Butyldiphenylsilyloxymethyl)-1-phenylcyclopropyl]ethyl}-5-methyl-1,3,4-oxadiazole (Prep 46-3)

A triethyl orthoacetate solution (3 ml) of compound Prep 46-2 (400 mg) was heated to reflux for 65 hours. After the reaction solution was allowed to cool at room temperature, water was added and potassium carbonate was added until it reached a saturation condition. The obtained mixture was then extracted with ethyl acetate and the organic layer was dried over magnesium sulfate and then the solvent was distilled away under reduced pressure. The residue was purified by silica gel column chromatography (n-heptane: ethyl acetate) to obtain the title compound (381.4 mg).

¹H-NMR (400 MHz, CDCl₃) δ (ppm): 0.42-4.20 (m, 9H), 1.09 (s, 9H), 2.42 (s, 3H), 7.08-7.76 (m, 15H).

### (4) 2-[2-(5-Methyl-1,3,4-oxadiazol-2-yl)ethyl]-2-phenylcyclopropanecarboxylic acid (Prep 46)

The compound was synthesized from compound Prep 46-3 by the same processes as in Production Examples 1-(6), (7), (8).

MS [M+H]⁺=273

### Production Example 47

### Synthesis of 2-[(3,4-dimethoxyphenyl)sulfonylmethyl]-2-phenylcyclopropanecarboxylic acid (Prep 47)

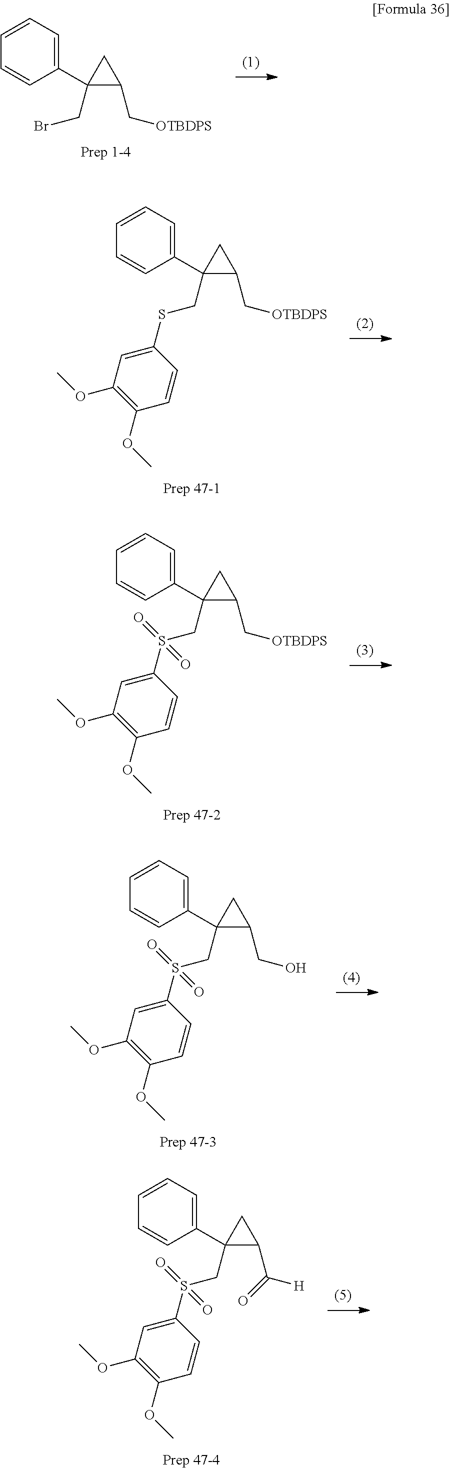

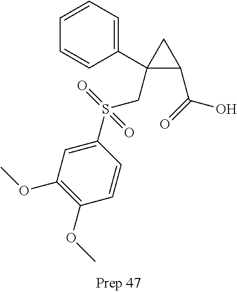

### (1) tert-Butyl({2-[(3,4-dimethoxyphenyl)thiomethyl]-2-phenylcyclopropyl}methoxy)diphenylsilane (Prep 47-1)

To a THF solution (15 ml) of 3,4-dimethoxythiophenol (195 mg), sodium hydride (60% oil dispersion, 45.8 mg) was slowly added and the obtained mixture was stirred for 5 minutes and then a THF solution (5 ml) of compound Prep 1-4 (500 mg) was added and the obtained mixture was stirred at room temperature for 1 hour. To the reaction solution, a saturated sodium bicarbonate solution was added and the obtained mixture was extracted with ethyl acetate. The organic layer obtained was washed with a saturated saline. The organic layer was dried over anhydrous magnesium sulfate and filtered. The filtrate was concentrated under reduced pressure and the residue was purified by silica gel column chromatography (n-heptane:ethyl acetate) to obtain the title compound (429 mg).

¹H-NMR (400 MHz, CDCl₃) δ (ppm): 0.68 (dd, J=6.0, 5.2 Hz, 1H), 1.09 (s, 9H), 1.13-1.18 (m, 1H), 1.45-1.53 (m, 1H), 3.16 (d, J=12.4 Hz, 1H), 3.70 (dd, J=11.2, 9.6 Hz, 1H), 3.84 (s, 3H), 3.86 (s, 3H), 3.87 (d, J=10.8 Hz, 1H), 4.00 (dd, J=11.2, 9.6 Hz, 1H), 6.68-6.92 (m, 5H), 7.19-7.44 (m, 10H), 7.67 (m, 3H).

### (2) tert-Butyl({2-[(3,4-dimethoxyphenyl)sulfonylmethyl]-2-phenylcyclopropyl}methoxy)diphenylsilane (Prep 47-2)

To a chloroform solution (15 ml) of compound Prep 47-1 (429 mg), m-chloroperbenzoic acid (325 mg) was slowly added at 0° C. and the obtained mixture was stirred at room temperature for 2 hours. To the reaction solution, a saturated sodium thiosulfate aqueous solution was added and then the obtained mixture was extracted with ethyl acetate. The organic layer obtained was washed with a saturated saline. The organic layer was dried over anhydrous magnesium sulfate and filtered. The filtrate was concentrated under reduced pressure and the residue was purified by silica gel column chromatography (n-heptane: ethyl acetate) to obtain the title compound (486 mg).

¹H-NMR (400 MHz, CDCl₃) δ (ppm): 1.08 (s, 9H), 1.14 (t, J=6.0 Hz, 1H), 1.24-1.28 (m, 1H), 1.34-1.39 (m, 1H), 3.36 (d, J=15.2 Hz, 1H), 3.53 (dd, 1H, J=11.6, 9.2 Hz, 1H), 3.77 (s, 3H), 4.02 (s, 3H), 4.10 (dd, J=11.6, 9.2 Hz, 1H), 6.77-6.80 (m, 1H), 6.99-7.02 (m, 1H), 7.10-7.15 (m, 5H), 7.26-7.45 (m, 3H), 7.58-7.67 (m, 9H).

### (3) {2-[(3,4-Dimethoxyphenyl)sulfonylmethyl]-2-phenylcyclopropyl}methanol (Prep 47-3)

To a THF solution (20 ml) of compound Prep 47-2 (486 mg), tetrabutylammonium fluoride (1 M THF solution: 2.43 ml) was added dropwise at 0° C. and the obtained mixture was stirred at room temperature for 1 hour. The reaction solution was concentrated under reduced pressure and the residue was purified by silica gel column chromatography (n-heptane: ethyl acetate) to obtain the title compound (219 mg).

¹H-NMR (400 MHz, CDCl₃) δ (ppm): 0.74 (t, J=5.2 Hz, 1H), 1.05 (dd, J=9.2, 4.8 Hz, 1H), 1.85-1.94 (m, 1H), 3.44 (d, J=15.2 Hz, 1H), 3.52 (t, 1H, J=10.8 Hz, 1H), 3.81 (s, 3H), 3.90 (s, 3H), 3.95 (d, J=15.2 Hz, 1H), 6.74-6.77 (m, 1H), 6.96-6.98 (m, 1H), 7.09-7.17 (m, 3H), 7.21-7.27 (m, 3H).

### (4) 2-[(3,4-Dimethoxyphenyl)sulfonylmethyl]-2-phenylcyclopropanecarbaldehyde (Prep 47-4)

To a dichloromethane solution (5 ml) of compound Prep 47-3 (486 mg), a Dess-Martin reagent (1.14 g) was added at 0° C. and the obtained mixture was stirred at room temperature for 1 hour. To the reaction solution, a saturated sodium bicarbonate solution was added and extracted with ethyl acetate. The organic layer obtained was washed with water and a saturated saline. The organic layer was dried over anhydrous magnesium sulfate and filtered. The filtrate was concentrated under reduced pressure and the residue was purified by silica gel column chromatography (n-heptane: ethyl acetate) to obtain the title compound (293 mg).

¹H-NMR (400 MHz, CDCl₃) δ (ppm): 1.53-1.62 (m, 1H), 1.77 (dd, J=6.2, 5.2 Hz, 1H), 2.62-2.67 (m, 1H), 3.62 (d, J=14.4 Hz, 1H), 3.79 (d, J=14.4 Hz, 1H), 3.88 (s, 3H), 3.92 (s, 3H), 6.85-6.87 (m, 1H), 7.09-7.11 (m, 1H), 7.24-7.33 (m, 4H), 7.37-7.42 (m, 2H).

### (5) 2-[(3,4-Dimethoxyphenyl)sulfonylmethyl)-2-phenylcyclopropanecarboxylic acid (Prep 47)

To an acetone-water solution (34.8 ml-14.9 ml) of compound Prep 47-4 (293 mg), 2-methyl-2-butene (917 μl), sodium dihydrogen phosphate (195 mg) and sodium chlorite (147 mg) were added at room temperature and the obtained mixture was stirred for 2 hours. To the reaction solution, a saturated ammonia chloride aqueous solution was added and the obtained mixture was extracted with ethyl acetate. The organic layer obtained was washed with a saturated saline, dried over anhydrous magnesium sulfate and filtered. The filtrate was concentrated under reduced pressure to obtain the title compound (58 mg).

MS [M+H]⁺=424

### Example 1

### Synthesis of N-phenyl-2-[3,4-dimethoxyphenyl)oxymethyl]-2-phenylcyclopropanecarboxamide (1)

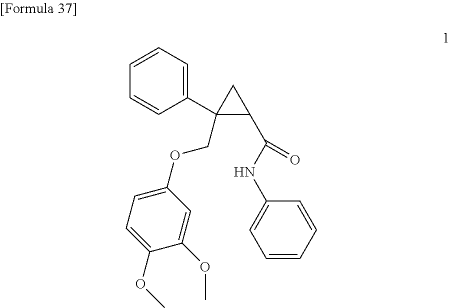

To a DMF solution (2 ml) of carboxylic acid Prep 1 (32.8 mg) and aniline (18.2 μl), triethylamine (41.8 μl) and HBTU (56.9 mg) were added while stirring at room temperature. After the reaction solution had been stirred at room temperature for 4 hours, water was added to the reaction solution. The obtained mixture was extracted with ethyl acetate and the organic layer was dried over magnesium sulfate and then concentrated under reduced pressure. The residue was purified by NH-silica gel column chromatography (n-heptane: ethyl acetate) to obtain the title compound (30 mg).

¹H-NMR (400 MHz, CDCl₃) δ (ppm): 1.54-1.59 (m, 1H), 1.87 (t, J=5.6 Hz, 1H), 2.04-2.10 (m, 1H), 3.62 (s, 3H), 3.77 (s, 3H), 4.25 (d, J=9.6 Hz, 1H), 4.53 (d, J=9.6 Hz, 1H), 6.32-6.34 (m, 2H), 6.65-6.68 (m, 1H), 7.05-7.09 (m, 1H), 7.25-7.29 (m, 3H), 7.33-7.36 (m, 2H), 7.43-7.47 (m, 4H), 7.64 (brs, 1H).

### Example 2

### Synthesis of N-methyl-N-phenyl-2-[(3,4-dimethoxyphenyl)oxymethyl]-2-phenylcyclopropanecarboxamide (2)

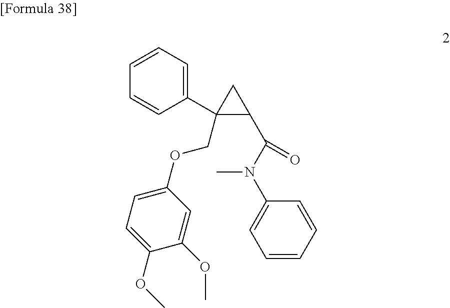

To a DMF solution (2 ml) of carboxylic acid Prep 1 (32.8 mg) and N-methylaniline (21.7 μl), triethylamine (41.8 μl) and HBTU (56.9 mg) were added while stirring at room temperature. After the reaction solution had been stirred at room temperature for 4 hours, to the reaction solution, water was added and the obtained mixture was extracted with ethyl acetate. The organic layer was dried over magnesium sulfate and then concentrated under reduced pressure. The residue was purified by NH-silica gel column chromatography (n-heptane:ethyl acetate) to obtain the title compound (16 mg).

¹H-NMR (400 MHz, CDCl₃) δ (ppm): 1.22-1.25 (m, 1H), 1.86 (t, J=5.2 Hz, 1H), 1.95-1.99 (m, 1H), 3.30 (m, 1H), 3.80 (s, 3H), 3.83 (s, 3H), 4.28 (d, J=9.6 Hz, 1H), 4.53 (d, J=9.6 Hz, 1H), 6.41 (dd, J=8.8, 2.8 Hz, 1H), 6.47 (d, J=2.8 Hz, 1H), 6.74 (d, J=8.8 Hz, 1H), 7.04-7.06 (m, 2H), 7.15-7.16 (m, 3H), 7.31-7.36 (m, 1H), 7.39-7.47 (m, 4H).

### Example 3

### Synthesis of N-(5-fluoropyridin-2-yl)-2-[(3,4-dimethoxyphenyl)oxymethyl]-2-phenylcyclopropanecarboxamide (3)

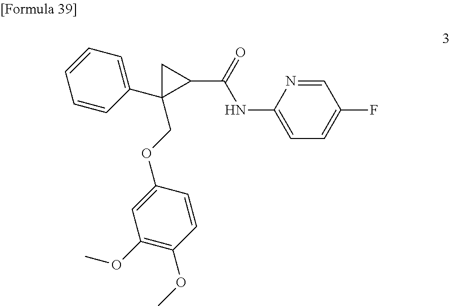

To a THF solution (500 μl) of trichloromethyl chloroformate (18.4 μl), a THF solution (500 μl) of carboxylic acid Prep 1 (50.0 mg) and triethylamine (84.9 μl) were added dropwise and the obtained mixture was stirred for 5 minutes and then 2-amino-5-fluoropyridine (59.6 mg) was added and the obtained mixture was stirred at room temperature overnight. To the reaction mixture, a saturated sodium bicarbonate aqueous solution was added and the obtained mixture was subjected to liquid separation and extraction with ethyl acetate. From the organic layer obtained, the solvent was distilled away by a nitrogen spray apparatus. The obtained residue was dissolved in DMF (1 ml) and subjected to LC-MS fractionation. The mass fraction of the desired substance obtained was dried by a nitrogen spray dryer to obtain the title compound (10.9 mg).

MS [M+H]⁺=423

### Example 4

### Synthesis of (1R,2S)—N-(5-cyanopyridin-2-yl)-2-[(3,4-dimethoxyphenyl)oxymethyl]-2-phenylcyclopropanecarboxamide (4)

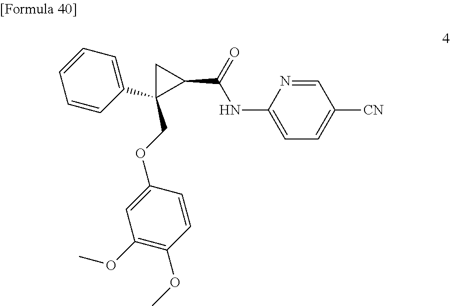

To a dioxane solution (6 ml) of trichloromethyl chloroformate (113 μl), a dioxane solution (4 ml) of carboxylic acid Prep 1 (300 mg) and triethylamine (255 μl) was added dropwise and the obtained mixture was stirred for 15 minutes and then a dioxane solution (4 ml) of 2-amino-5-cyanopyridine (545 mg) was added and the obtained mixture was stirred at room temperature overnight. To the reaction mixture, a saturated sodium bicarbonate aqueous solution was added. The organic layer obtained by liquid separation and extraction with ethyl acetate was dried over anhydrous magnesium sulfate and the filtrate was concentrated under reduced pressure. The residue was purified by NH-silica gel column chromatography (n-heptane:ethyl acetate) to obtain a racemic form (226 mg) of the title compound. Fractionation was performed by CHIRALPAKTM AD-H (2 cm×25 cm, mobile layer; ethanol) manufactured by Daicel Chemical Industries, Ltd. to obtain the title compound (80 mg).

¹H-NMR (400 MHz, CDCl₃) δ (ppm): 1.62-1.65 (m, 1H), 1.94 (t, J=5.6 Hz, 1H), 2.14 (dd, J=6.0 Hz, 8.0 Hz, 1H), 3.67 (s, 3H), 3.77 (s, 3H), 4.22 (d, J=9.6 Hz, 1H), 4.48 (d, J=10.4 Hz, 1H), 6.25-6.29 (m, 2H), 6.64 (d, J=8.4 Hz, 1H), 7.28-7.46 (m, 5H), 7.84 (dd, J=1.6 Hz, 8.8 Hz, 1H), 8.20 (d, J=8.4 Hz, 1H), 8.52 (d, J=1.6 Hz, 1H), 8.70 (s, 1H).

MS [M+H]⁺=430

### Example 5

### Synthesis of N-(4-methyl-1,3-thiazol-2-yl)-2-[(3,4-dimethoxyphenyl)oxymethyl]-2-phenylcyclopropanecarboxamide (5)

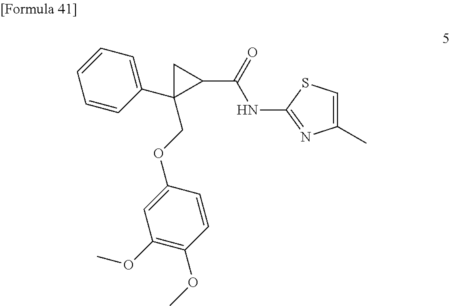

To a DMF solution (1 ml) of carboxylic acid Prep 1 (30 mg), HATU (41.7 mg), 2-amino-4-methylthiazole (52.2 mg) and N,N-diisopropylethylamine (34 μl) were successively added and the obtained mixture was stirred at room temperature overnight. Thereafter, the temperature of the reaction solution was increased to 60° C. and the reaction solution was stirred overnight. The reaction mixture was subjected to LC-MS fractionation. The mass faction of the desired substance obtained was dried by a nitrogen spray dryer to obtain the title compound (19.6 mg).

MS [M+H]⁺=425

### Example 6

### Synthesis of N-(5-chloropyridin-2-yl)-2-[(3,4-dimethoxyphenyl)oxymethyl]-2-phenylcyclopropanecarboxamide (6)

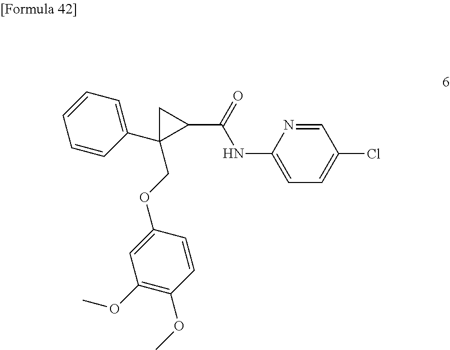

To a DMF solution (1 ml) of carboxylic acid Prep 1 (30 mg), HATU (41.7 mg), 2-amino-5-chloropyridine (23.5 mg) and N,N-diisopropylethylamine (34 μl) were successively added and the obtained mixture was stirred at room temperature overnight. The reaction mixture was subjected to LC-MS fractionation. The mass faction of the desired substance obtained was dried by a nitrogen spray dryer to obtain the title compound (5.3 mg).

MS [M+H]⁺=439

The compounds of Examples 7 to 19 were each synthesized by condensing carboxylic acid Preps 1 with an amine according to the process of Example 3.

TABLE 6

| Example | Structural formula, MS |
|---|---|
| 7 | 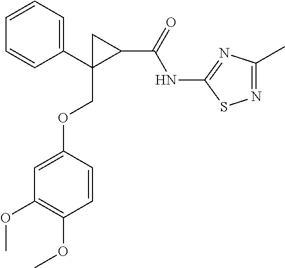 MS [M + H]⁺ = 426 |
| 8 | 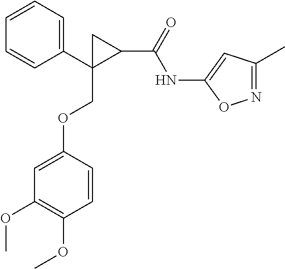 MS [M + H]⁺ = 409 |
| 9 | 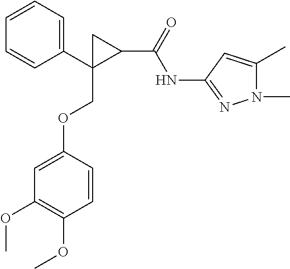 MS [M + H]⁺ = 422 |
| 10 | 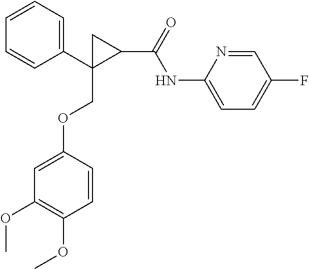 MS [M + H]⁺ = 423 |
| 11 | 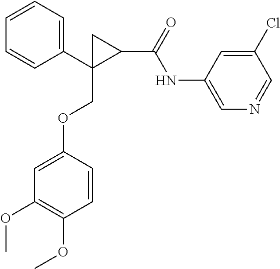 MS [M + H]⁺ = 439 |
| 12 | 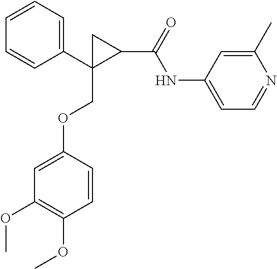 MS [M + H]⁺ = 419 |
| 13 | 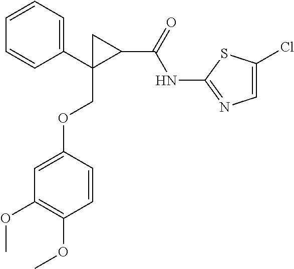 MS [M + H]⁺ = 445 |
| 14 | 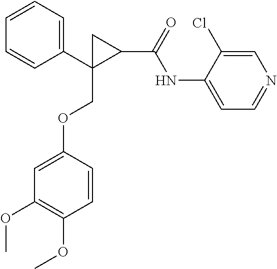 MS [M + H]⁺ = 439 |
| 15 | 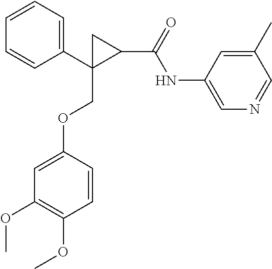 MS [M + H]⁺ = 419 |
| 16 | 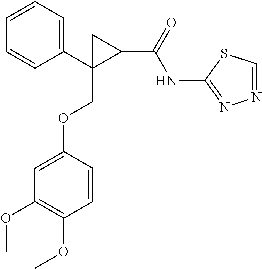 MS [M + H]⁺ = 412 |
| 17 | 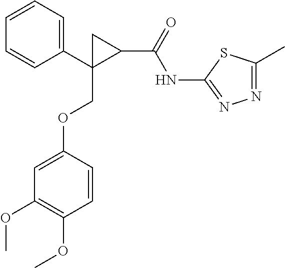 MS [M + H]⁺ = 426 |
| 18 | 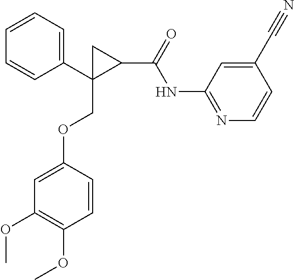 MS [M + H]⁺ = 430 |
| 19 | 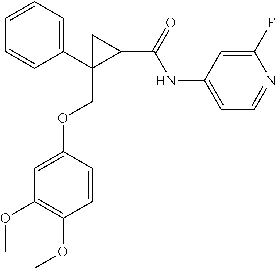 MS [M + H]⁺ = 423 |

The compounds of Examples 20 to 33 were each synthesized by condensing carboxylic acid Preps 1 with an amine according to the process of Example 3.

TABLE 7

| Example | Structural formula, MS |
|---|---|
| 20 | 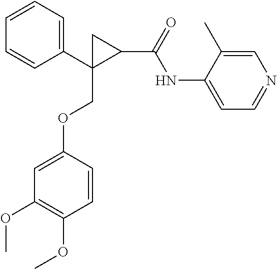 MS [M + H]⁺ = 419 |
| 21 | 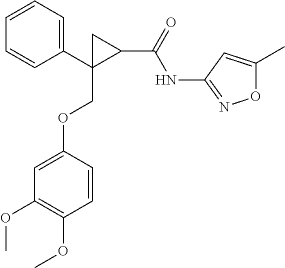 MS [M + H]⁺ = 409 |
| 22 | 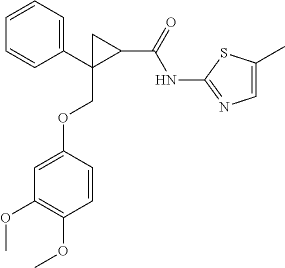 MS [M + H]⁺ = 425 |
| 23 | 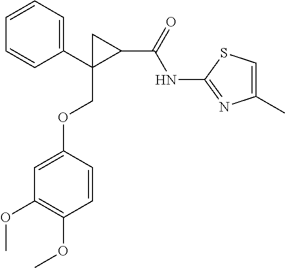 MS [M + H]⁺ = 425 |
| 24 | 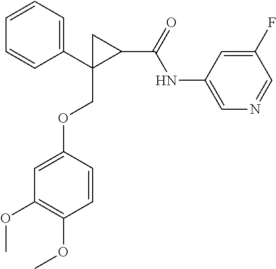 MS [M + H]⁺ = 423 |
| 25 | 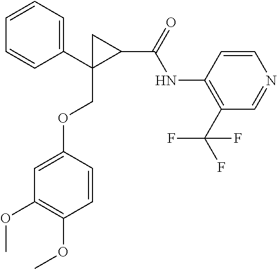 MS [M + H]⁺ = 473 |
| 26 | 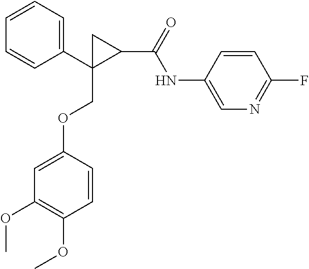 MS [M + H]⁺ = 423 |
| 27 | 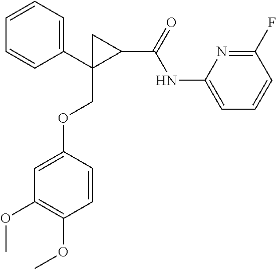 MS [M + H]⁺ = 423 |
| 28 | 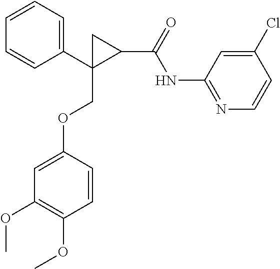 MS [M + H]⁺ = 439 |
| 29 | 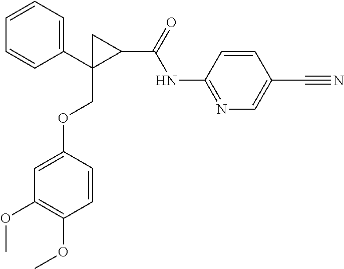 MS [M + H]⁺ = 430 |
| 30 | 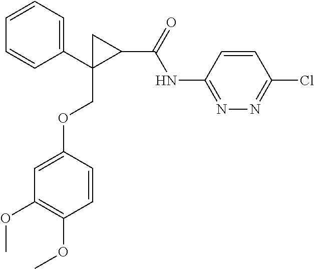 MS [M + H]⁺ = 440 |
| 31 | 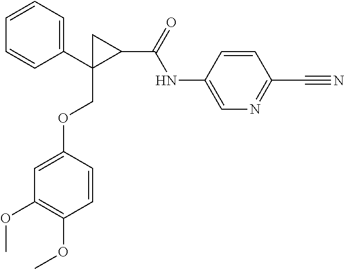 MS [M + H]⁺ = 430 |
| 32 | 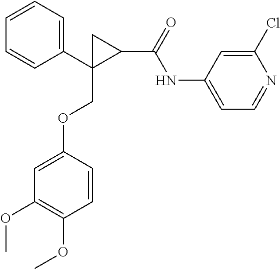 MS [M + H]⁺ = 439 |
| 33 | 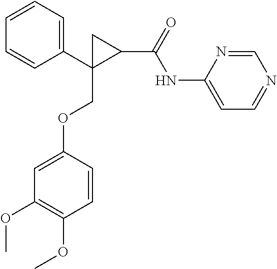 MS [M + H]⁺ = 406 |

The compounds of Examples 34 to 43 were synthesized by condensing carboxylic acid Preps 1 with an amine by use of HBTU. In Example 41, chiral resolution was performed.

TABLE 8

| Example | Structural formula, MS |
|---|---|
| 34 | 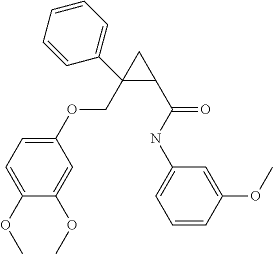 MS [M + H]⁺ = 434 |
| 35 | 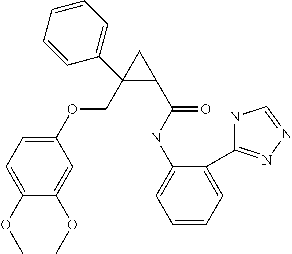 MS [M + H]⁺ = 471 |
| 36 | 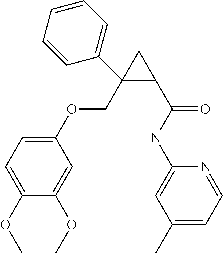 MS [M + H]⁺ = 419 |
| 37 | 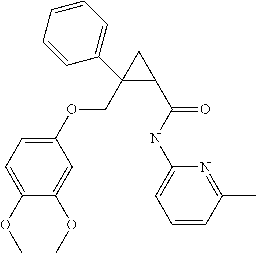 MS [M + H]⁺ = 419 |
| 38 | 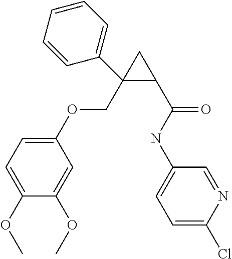 MS [M + H]⁺ = 434 |
| 39 | 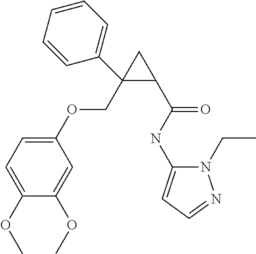 MS [M + H]⁺ = 422 |
| 40 | 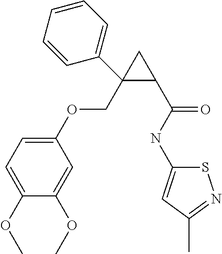 MS [M + H]⁺ = 425 |
| 41 | 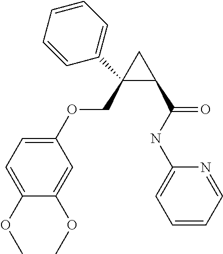 MS [M + H]⁺ = 405 |
| 42 | 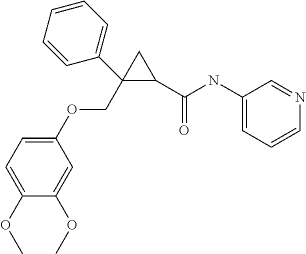 MS [M + H]⁺ = 405 |
| 43 | 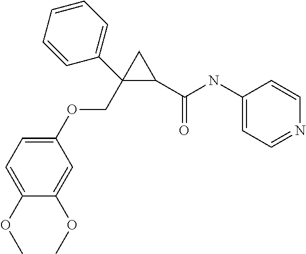 MS [M + H]⁺ = 405 |

### Example 44

### Synthesis of 2-{[3,4-dimethoxyphenoxy]methyl]-2-phenylcyclopropyl}-5-fluoro-1H-benzimidazole (44)

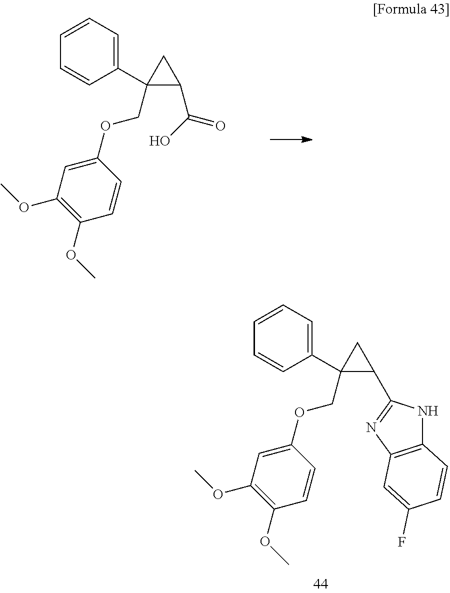

To a dichloromethane solution (6 ml) of carboxylic acid Prep 1 (100 mg), oxalyl chloride (52.4 μl) and catalytic amount of DMF were added and the obtained mixture was stirred at room temperature for 1 hour. The reaction mixture was concentrated under reduced pressure to obtain a crude acid chloride. To a THF solution of 3,4-diamino-1-fluorobenzene (51.9 mg), triethylamine (106 μl) was added, a THF solution of the crude acid chloride was added to allow to react at room temperature for 30 minutes, and then further stirred for 1 hour under heating to reflux. The reaction mixture was concentrated under reduced pressure and acetic acid (6 ml) was added and the obtained mixture was stirred for 2 hours under heating to reflux. The reaction mixture was concentrated under reduced pressure and ethyl acetate and a saturated sodium bicarbonate aqueous solution were added to carry out liquid separation and extraction. The organic layer obtained was dried over magnesium sulfate and concentrated under reduced pressure. The obtained residue was purified by silica gel column chromatography (n-heptane:ethyl acetate) to obtain the title compound (80 mg).

¹H-NMR (400 MHz, CDCl₃) δ (ppm): 1.73 (dd, J=8.8, 5.6 Hz, 1H), 2.00 (t, J=6.0 Hz, 1H), 2.66 (8.8, 6.0 Hz, 1H), 3.64 (s, 3H), 3.80 (s, 1H), 3.99 (d, J=10.0 Hz, 1H), 4.38 (d, J=10.0 Hz, 1H), 6.12-6.15 (m, 2H), 6.55-6.58 (m, 1H), 6.92-6.99 (m, 1H), 7.28-7.95 (m, 7H), 9.66 (brs, 1H).

### Example 45

### Synthesis of 3-{2-[(3,4-dimethoxyphenoxy)methyl]-2-phenylcyclopropyl}-5-phenyl-1H-1,2,4-triazole (45)

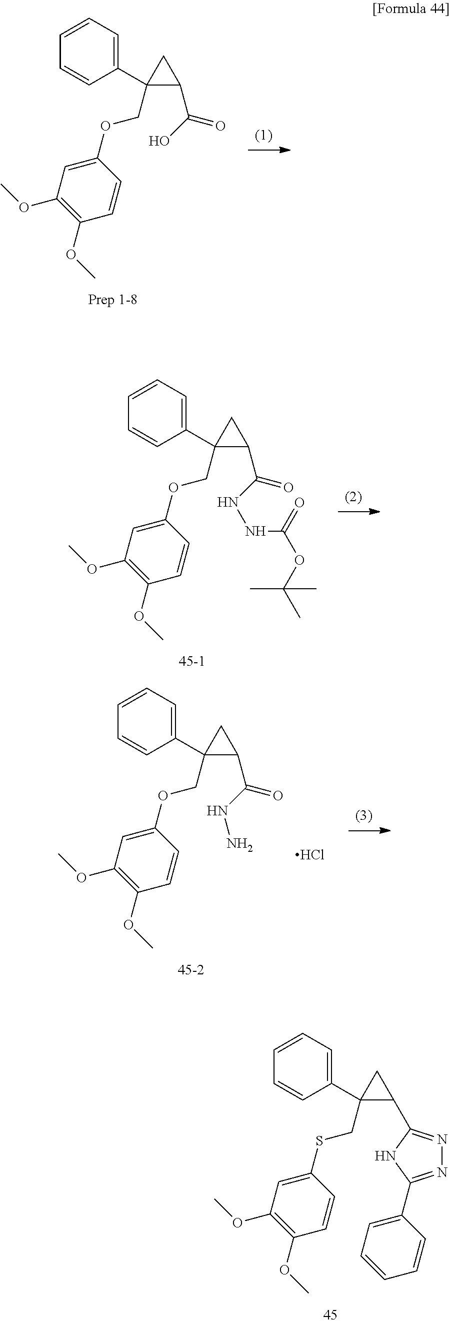

### (1) tert-Butyl 2-({(2-[(3,4-dimethoxyphenoxy)methyl]-2-phenylcyclopropyl}carbonyl)hydrazine carboxylate (45-1)

To a DMF solution (4 ml) of carboxylic acid Prep 1 (300 mg), WSC (210 mg), HOBt (148 mg) and triethylamine (318 μl) were added and the obtained mixture was stirred at room temperature overnight. To the reaction mixture, water was added and the obtained mixture was subjected to liquid separation and extraction with diethyl ether. The organic layer obtained was dried over magnesium sulfate and concentrated under reduced pressure. The obtained residue was purified by NH-silica gel column chromatography (: n-heptane: ethyl acetate) to obtain the title compound (405 mg).

MS [M+H]⁺=443.

### (2) 2-[(3,4-Dimethoxyphenyl oxy)methyl]-2-phenylcyclopropanecarbohydrazide hydrochloride (45-2)

A 4 N hydrochloric acid-ethyl acetate solution (5 ml) of the compound 45-1 (405 mg) was stirred at room temperature for 1.5 hours. The reaction mixture was concentrated under reduced pressure to obtain a crude product.

MS [M+H]⁺=343.

### (3) 3-{2-[(3,4-Dimethoxyphenoxy)methyl]-2-phenylcyclopropyl}-5-phenyl-1H-1,2,4-triazole (45)

To a DMF (3 ml)-ethanol (3 ml) solution of ethyl benzimidate hydrochloride (199 mg), imidazole (583 mg) was added and the obtained mixture was stirred at room temperature for 5 minutes. To the reaction mixture, a DMF-ethanol solution of the compound 45-2 (405 mg) was added dropwise and the obtained mixture was stirred at room temperature overnight. The temperature of the reaction mixture was increased to 100° C. and the reaction solution was stirred for 5 hours. To the reaction mixture, water and diethyl ether were added to carry out liquid separation and extraction. The organic layer obtained was dried over magnesium sulfate and concentrated under reduced pressure. The obtained residue was purified by silica gel column chromatography (n-heptane: ethyl acetate) to obtain the title compound (250 mg).

MS [M+H]⁺=428.

The compounds of Examples 46 to 56 were each synthesized by using carboxylic acid Preps 8 to 15 and an amine, with the help of a condensing agent, and, if necessary, converted to salts thereof. The compound of Example 56 was obtained by chiral resolution of a racemic form.

TABLE 9

| Example | Structural formula | NMR and/or MS |
|---|---|---|
| 46 | 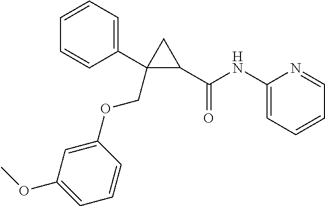 | ¹H-NMR (400 MHz, CDCl₃) δ (ppm): 1.59 (dd, J = 8.0, 4.8 Hz, 1H), 1.90 (dd, J = 6.0, 4.8 Hz, 1H), 2.10 (dd, J = 8.0, 6.0 Hz, 1H), 3.64 (s, 3H), 4.29 (d, J = 9.6 Hz, 1H), 4.48 (d, J = 9.6 Hz, 1H), 6.30 (t, J = 2.4 Hz, 1H), 6.35-6.43 (m, 2H), 6.97- 7.02 (m, 1H), 7.04 (d, J = 8.8 Hz, 1H), 7.24-7.29 (m, 1H), 7.31-7.37 (m, 2H), 7.43-7.48 (m, 2H), 7.60-7.66 (m, 1H), 8.06 (d, J = 8.4 Hz, 1H), 8.22-8.27 (m, 1H), 8.38 (s, 1H). MS [M + Na]⁺ = 397 |
| 47 | 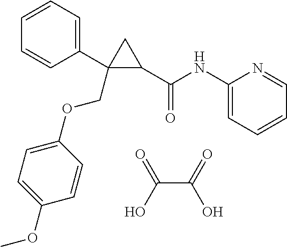 | ¹H-NMR (400 MHz, CD₃OD) δ (ppm): 1.56 (dd, J = 8.0, 4.8 Hz, 1H), 1.83 (dd, J = 6.0, 4.8 Hz, 1H), 2.44 (dd, J = 8.0, 6.0 Hz, 1H), 3.65 (s, 3H), 4.25 (d, J = 10.0 Hz, 1H), 4.47 (d, J = 10.0 Hz, 1H), 6.66 (s, 4H), 7.19-7.28 (m, 2H), 7.31- 7.37 (m, 2H), 7.51-7.56 (m, 2H), 7.81 (d, J = 8.4 Hz, 1H), 7.86-7. (m, 1H), 8.24-8.28 (m, 1H). MS [M + H]⁺ = 375 (free base) |
| 48 | 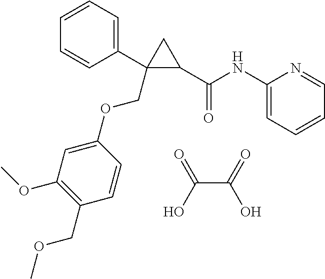 | ¹H-NMR (400 MHz, CD₃OD) δ (ppm): 1.59 (dd, J = 8.0, 5.2 Hz, 1H), 1.86 (dd, J = 6.0, 5.2 Hz, 1H), 2.46 (dd, J = 8.0, 6.0 Hz, 1H), 3.28 (s, 3H), 3.60 (s, 3H), 4.30 (s, 2H), 4.31 (d, J = 10.0 Hz, 1H), 4.56 (d, J = 10.0 Hz, 1H), 6.29 (d, J = 2.4 Hz, 1H), 6.34 (dd, J = 8.0, 2.4 Hz, 1H), 7.04 (d, J = 8.0 Hz, 1H), 7.15-7.20 (m, 1H), 7.04 (d, J = 8.0 Hz, 1H), 7.15-7.20 (m, 1H), 7.23-7.28 (m, 1H), 7.31-7.38 (m, 2H), 7.52-7.57 (m, 2H), 7.78-7.87 (m, 2H), 8.24-8.28 (m, 1H). MS [M + H]⁺ = 419 (free base) |
| 49 | 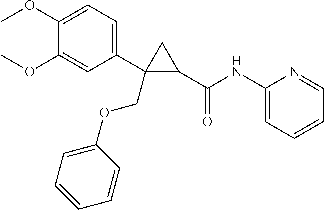 | ¹H-NMR (400 MHz, CDCl₃) δ (ppm): 1.58 (dd, J = 8.4, 5.2 Hz, 1H), 1.86 (t, J = 5.2 Hz, 1H), 2.04-2.10 (m, 1H), 3.87 (s, 3H), 3.88 (s, 3H), 4.33 (d, J = 10.0 Hz, 1H), 4.44 (d, J = 10.0 Hz, 1H), 6.74-6.87 (m, 4H), 6.96-7.05 (m, 3H), 7.11- 7.18 (m, 2H), 7.60-7.66 (m, 1H), 8.07 (d, J = 8.4 Hz, 1H), 8.23-8.27 (m, 1H), 8.49 (s, 1H). MS [M + Na]⁺ = 427 |
| 50 | 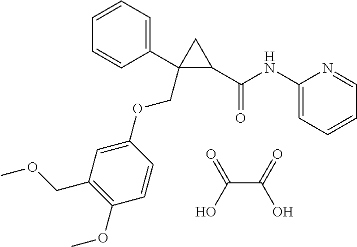 | ¹H-NMR (400 MHz, CD₃OD) δ (ppm): 1.57 (dd, J = 8.0, 5.2 Hz, 1H), 1.84 (dd, J = 6.0, 5.2 Hz, 1H), 2.44 (dd, J = 8.0, 6.0 Hz, 1H), 3.28 (s, 3H), 3.69 (s, 3H), 4.25 (d, J = 10.0 Hz, 1H), 4.27 (d, J = 12.4 Hz, 1H), 4.31 (d, J = 12.4 Hz, 1H), 4.49 (d, J = 10.0 Hz, 1H), 4.86 (s, 2H), 6.65 (dd, J = 8.8, 2.8 Hz, 1H), 6.71 (d, J = 8.8 Hz, 1H), 6.71 (d, J = 2.8 Hz, 1H), 7.17-7.21 (m, 1H), 7.22-7.28 (m, 1H), 7.31-7.37 (m, 2H), 7.52-7.56 (m, 2H), 7.80 (d, J = 8.8 Hz, 1H), 7.85- 7.91 (m, 1H), 8.24-8.27 (m, 1H). MS [M + H]⁺ = 419 (free base) |
| 51 | 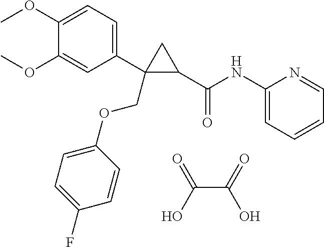 | ¹H-NMR (400 MHz, CD₃OD) δ (ppm): 1.56 (dd, J = 8.0, 4.8 Hz, 1H), 1.80 (dd, J = 6.0, 4.8 Hz, 1H), 2.41 (dd, J = 8.0, 6.0 Hz, 1H), 3.81 (s, 3H), 3.85 (s, 3H), 4.28 (d, J = 10.0 Hz, 1H), 4.45 (d, J = 10.0 Hz, 1H), 6.69-6.75 (m, 2H), 6.80- 6.87 (m, 2H), 6.91 (d, J = 8.4 Hz, 1H), 7.08 (dd, J = 8.4, 2.0 Hz, 1H), 7.16 (d, J = 2.0 Hz, 1H), 7.18-7.20 (m, 1H), 7.81-7.88 (m, 2H), 8.25-8.29 (m, 1H). MS [M + H]⁺ = 423 (free base) |
| 52 | 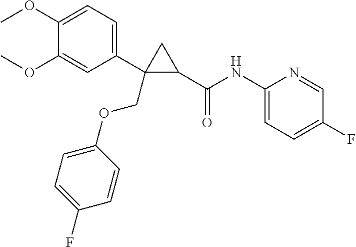 | ¹H-NMR (400 MHz, CDCl₃) δ (ppm): 1.58 (dd, J = 8.4, 5.2 Hz, 1H), 1.84 (t, J = 5.2 Hz, 1H), 2.02-2.07 (m, 1H), 3.88 (s, 3H), 3.90 (s, 3H), 4.27 (d, J = 9.6 Hz, 1H), 4.50 (d, J = 9.6 Hz, 1H), 6.65-6.71 (m, 2H), 6.79-6.86 (m, 3H), 6.88 (dd, J = 8.4, 2.0 Hz, 1H), 7.01 (d, J = 2.0 Hz, 1H), 7.35- 7.41 (m, 1H), 8.08 (dd, J = 5.2, 4.0 Hz, 1H), 8.12 (d, J = 3.2 Hz, 1H), 8.30 (s, 1H). MS [M + H]⁺ = 441 (free base) |
| 53 | 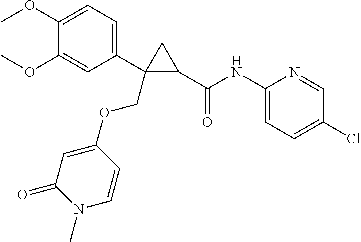 | ¹H-NMR (400 MHz, CDCl₃) δ (ppm): 1.61 (dd, J = 8.0, 5.2 Hz, 1H), 1.88 (dd, J = 6.0, 5.2 Hz, 1H), 2.09 (dd, J = 8.0, 6.0 Hz, 1H), 3.39 (s, 3H), 4.27 (d, J = 10.0 Hz, 1H), 4.41 (d, J = 10.0 Hz, 1H), 5.69 (dd, J = 7.6, 2.4 Hz, 1H), 5.81 (d, J = 2.4 Hz, 1H), 6.96 (d, J = 7.6 Hz, 1H), 7.24-7.42 (m, 5H), 7.63 (dd, J = 8.4, 2.4 Hz, 1H), 8.10 (d, J = 8.4 Hz, 1H), 8.22 (d, J = 2.4 Hz, 1H), 8.28 (s, 1H). MS [M + Na]⁺ = 432 |
| 54 | 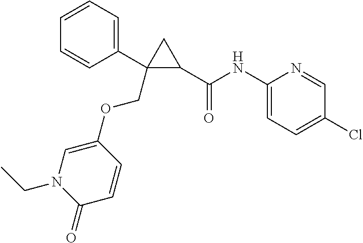 | ¹H-NMR (400 MHz, CDCl₃) δ (ppm): 1.25 (t, J = 7.2 Hz, 3H), 1.56-1.62 (m, 1H), 1.84 (t, J = 5.6 Hz, 1H), 2.09 (dd, J = 7.6, 5.6 Hz, 1H), 3.76-3.89 (m, 2H), 4.16 (d, J = 9.6 Hz, 1H), 4.31 (d, J = 9.6 Hz, 1H), 6.38 (d, J = 9.6 Hz, 1H), 6.56 (d, J = 3.2 Hz, 1H), 6.97 (dd, J = 9.6, 3.2 Hz, 1H), 7.24- 7.45 (m, 5H), 7.66 (dd, J = 8.8, 2.4 Hz, 1H), 8.12 (d, J = 8.8 Hz, 1H), 8.24 (d, J = 2.4 Hz, 1H), 8.33 (s, 1H). MS [M + H]⁺ = 424 |
| 55 | 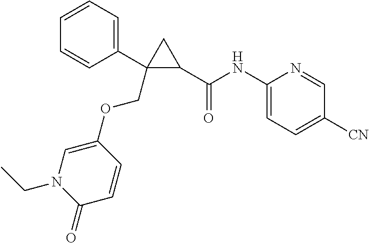 | ¹H-NMR (400 Hz, CDCl₃) δ (ppm): 1.26 (t, J = 7.2 Hz, 3H), 1.65 (dd, J = 8.0, 4.8 Hz, 1H), 1.84 (dd, J = 6.0, 4.8 Hz, 1H), 2.14 (dd, J = 8.0, 6.0 Hz, 1H), 3.76-3.91 (m, 2H), 4.17 (d, J = 9.6 Hz, 1H), 4.30 (d, J = 9.6 Hz, 1H), 6.38 (d, J = 9.6 Hz, 1H), 6.66 (d, J = 3.2 Hz, 1H), 6.95 (dd, J = 9.6, 3.2 Hz, 1H), 7.24-7.45 (m, 5H), 7.93 (dd, J = 8.8, 2.4 Hz, 1H), 8.48 (d, J = 8.8 Hz, 1H), 8.57 (d, J = 2.4 Hz, 1H), 8.57 (s, 1H). MS [M + H]⁺ = 415 |
| 56 | 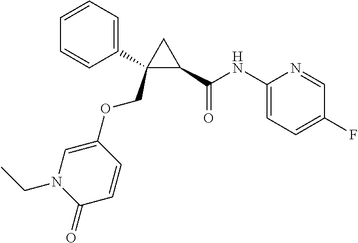 | ¹H-NMR (400 MHz, CDCl₃) δ (ppm): 1.25 (t, J = 7.2 Hz, 3H), 1.58 (dd, J = 8.4, 5.2 Hz, 1H), 1.84 (t, J = 5.4 Hz, 1H), 2.11 (dd, J = 7.8, 5.8 Hz, 1H), 3.75-3.85 (m, 2H), 4.18 (d, J = 9.6 Hz, 1H), 4.31 (d, J = 9.6 Hz, 1H), 6.37 (d, J = 10.4 Hz, 1H), 6.66 (d, J = 2.8 Hz, 1H), 6.97 (dd, J = 10.0, 3.2 Hz, 1H), 7.25-7.46 (m, 6H), 8.12 (d, J = 2.8 Hz, 1H), 8.12- 8.18 (m, 1H), 8.56 (brs, 1H). |

The compounds of Examples 57 and 58 were each synthesized by condensing carboxylic acid Prep 4 with an amine according to the process of Example 5.

TABLE 10

| Example | Structural formula, MS |
|---|---|
| 57 | 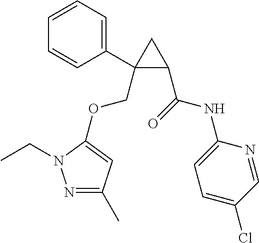 MS [M + H]⁺ = 411 |
| 58 | 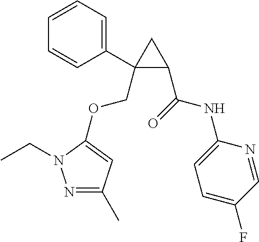 MS [M + H]⁺ = 395 |

The compounds of Examples 59 and 60 were each synthesized by condensing carboxylic acid Prep 24 with an amine according to the process of Example 5.

TABLE 11

| Example | Structural formula, MS |
|---|---|
| 59 | 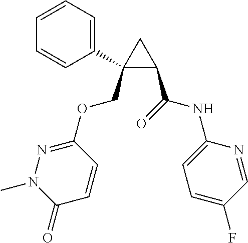 MS [M + Na]⁺ = 417 |
| 60 | 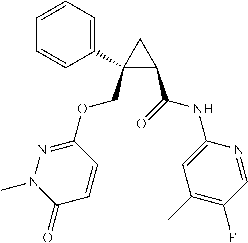 MS [M + Na]⁺ = 431 |

The compounds of Examples 61 to 64 were each synthesized from carboxylic acid Preps 25, 26, 28 and an amine by the same process as in Example 5.

TABLE 12

| Example | Structural formula, MS or NMR |
|---|---|
| 61 | 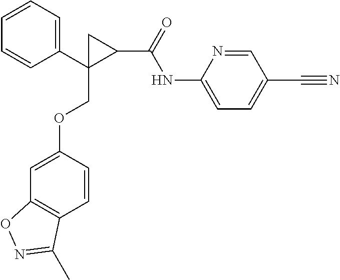 MS [M + H]⁺ = 425 |
| 62 | 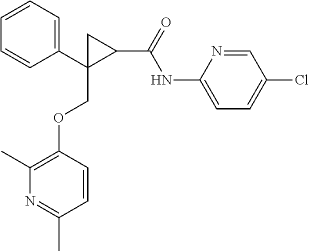 MS [M + H]⁺ = 408 |
| 63 | 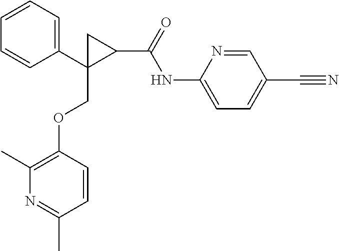 MS [M + H]⁺ = 399 |
| 64 | 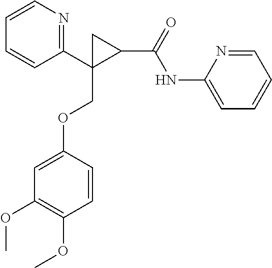 MS [M + H]⁺ = 406 |

The compounds of Examples 65 to 67 were each synthesized by condensing carboxylic acid Prep 27 and an amine according to the process of Example 5.

TABLE 13

| Example | Structural formula, MS |
|---|---|
| 65 | 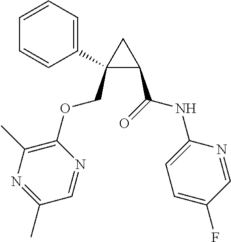 [M + Na]⁺ = 415 |
| 66 | 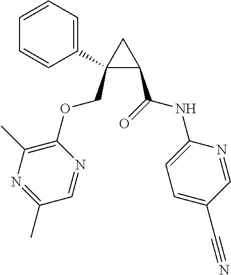 MS [M + Na]⁺ = 422 |
| 67 | 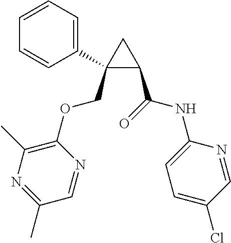 MS [M + Na]⁺ = 431 |

### Example 68

### Synthesis of (1R,2S)—N-(5-cyanopyridin-2-yl)-2-[(1-ethyl-3-methyl-1H-pyrazol-4-yl)oxymethyl]-2-(pyridin-2-yl)cyclopropanecarboxamide (68)

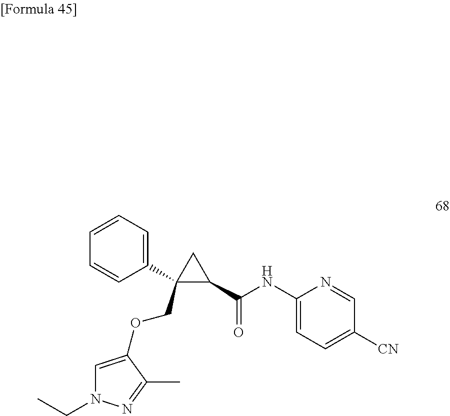

The carboxylic acid Prep 7 (565 mg) was dissolved in dichloromethane (6 ml) and oxalyl chloride (322 μl) and DMF (several drops) were added and the obtained mixture was stirred at room temperature for 2 hours. The reaction solution was concentrated under reduced pressure to obtain a crude acid chloride. 2-amino-5-cyanopyridine (337 mg) was suspended in THF (7 ml) and N,N-diisopropylethylamine (746 μl) was added and the temperature of the reaction solution was increased to 60° C. A THF solution (10 ml) of the crude acid chloride was added and the obtained mixture was stirred for 1 hour while maintaining the temperature. The reaction solution was concentrated under reduced pressure and water was added to the residue and the obtained mixture was extracted with ethyl acetate. The organic layer was successively washed with water, a saturated sodium bicarbonate aqueous solution and a saturated saline, and dried over anhydrous magnesium sulfate. After filtration, the filtrate was concentrated under reduced pressure. The residue was purified by silica gel column chromatography (n-heptane:ethyl acetate) to obtain the title compound (545 mg).

¹H-NMR (400 MHz, CDCl₃) δ (ppm): 1.34 (t, J=7.2 Hz, 3H), 1.62 (dd, J=8.0, 5.2 Hz, 1H), 1.88 (dd, J=6.0, 5.2 Hz, 1H), 1.89 (s, 3H), 2.11 (dd, J=8.0, 6.0 Hz, 1H), 3.90 (q, J=7.2 Hz, 2H), 4.11 (d, J=9.6 Hz, 1H), 4.29 (d, J=9.6 Hz, 1H), 6.84 (s, 1H), 7.24-732 (m, 1H), 7.44-7.49 (m, 2H), 7.42-7.47 (m, 2H), 7.90 (dd, J=8.4, 2.0 Hz, 1H), 8.28 (dd, J=8.4, 0.8 Hz, 1H), 8.52 (s, 1H), 8.55 (dd, J=2.0, 0.8 Hz, 1H).

MS [M+H]⁺=402

The compounds of Examples 69 to 73 were each synthesized by condensing carboxylic acid Prep 7 with an amine according to the process of Example 5.

TABLE 14

| Example | Structural formula | NMR and/or MS |
|---|---|---|
| 69 | 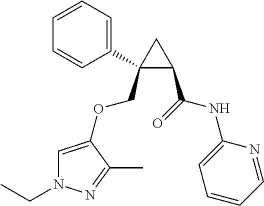 | ¹H-NMR (400 MHz, CDCl₃) δ (ppm): 1.32 (t, J = 7.4 Hz, 3H), 1.56 (dd, J = 7.8, 5.0 Hz, 1H), 1.85 (t, J = 5.4 Hz, 1H), 1.91 (s, 3H), 2.04-2.12 (m, 1H), 3.88 (q, J = 7.4 Hz, 2H), 4.15 (d, J = 9.6 Hz, 1H), 4.29 (d, J = 10.0 Hz, 1H), 6.85 (s, 1H), 7.01 (ddd, J = 11.2, 4.8, 1.2 Hz, 1H), 7.24-7.30 (m, 1H), 7.31- 7.38 (m, 2H), 7.42-7.48 (m, 2H), 7.65-7.72 (m, 1H), 8.15 (d, J = 8.4 Hz, 1H), 8.23-8.27 (m, 1H), 8.54 (brs, 1H). |
| 70 | 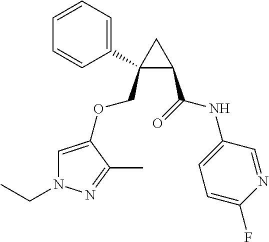 | ¹H-NMR (400 MHz, CDCl₃) δ (ppm): 1.35 (t, J = 7.2 Hz, 3H), 1.52-1.63 (m, 1H), 1.85 (t, J = 5.6 Hz, 1H), 1.96 (s, 3H), 2.05-2.12 (m, 1H), 3.92 (q, J = 7.2 Hz, 2H), 4.14 (d, J = 10.0 Hz, 1H), 4.33 (d, J = 9.6 Hz, 1H), 6.85-6.92 (m, 2H), 7.25- 7.32 (m, 1H), 7.32-7.39 (m, 2H), 7.42-7.47 (m, 2H), 7.64 (brs, 1H), 8.08-8.15 (m, 1H), 8.17-8.22 (m, 1H). |
| 71 | 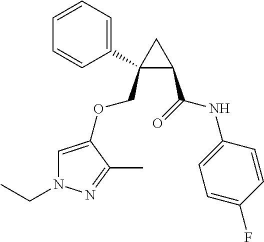 | ¹H-NMR (400 MHz, CDCl₃) δ (ppm): 1.34 (t, J = 7.4 Hz, 3H), 1.52-1.61 (m, 1H), 1.82 (t, J = 5.4 Hz, 1H), 1.97 (s, 3H), 2.02-2.08 (m, 1H), 3.91 (q, J = 7.4 Hz, 2H), 4.15 (d, J = 9.6 Hz, 1H), 4.34 (d, J = 9.6 Hz, 1H), 6.90 (s, 1H), 6.97-7.03 (m, 2H), 7.24-7.31 (m, 1H), 7.35 (t, J = 7.8 Hz, 2H), 7.40-7.47 (m, 4H), 7.55 (brs, 1H). |
| 72 | 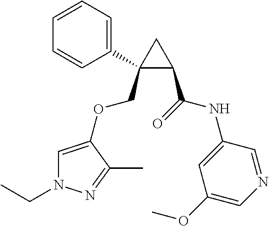 | ¹H-NMR (400 MHz, CDCl₃) δ (ppm): 1.32 (t, J = 7.4 Hz, 3H), 1.55 (dd, J = 8.0, 5.0 Hz, 1H), 1.83 (t, J = 5.4 Hz, 1H), 1.94 (s, 3H), 2.12 (dd, J = 8.0, 6.0 Hz, 1H), 3.79 (s, 3H), 3.88 (q, J = 7.4 Hz, 2H), 4.17 (d, J = 10.0 Hz, 1H), 4.35 (d, J = 9.6 Hz, 1H), 6.89 (s, 1H), 7.23-7.28 (m, 1H), 7.29-7.36 (m, 2H), 7.41 (brd, J = 7.6 Hz, 2H), 7.86 (brs, 1H), 8.04 (d, J = 2.4 Hz, 2H), 8.26 (brs, 1H). MS [M + H]⁺ = 407 |
| 73 | 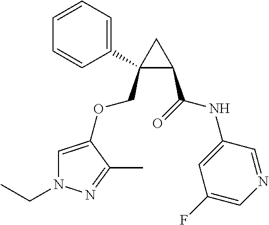 | ¹H-NMR (400 MHz, CDCl₃) δ (ppm): 1.34 (t, J = 7.4 Hz, 3H), 1.48-1.67 (m, 1H), 1.86 (t, J = 5.8 Hz, 1H), 1.95 (s, 3H), 2.08 (dd, J = 8.0, 6.0 Hz, 1H), 3.90 (q, J = 7.4 Hz, 2H), 4.11 (d, J = 9.6 Hz, 1H), 4.34 (d, J = 10.0 Hz, 1H), 6.87 (s, 1H), 7.14- 7.17 (m, 1H), 7.25-7.30 (m, 1H), 7.31-7.38 (m, 2H), 7.38- 7.44 (m, 2H), 8.06 (d, J = 6.0 Hz, 1H), 8.16 (s, 1H). |

The compounds of Examples 74 and 75 were each synthesized by condensing carboxylic acid Prep 5 with an amine according to the process of Example 68.

TABLE 15

| Example | Structural formula, MS |
|---|---|
| 74 | 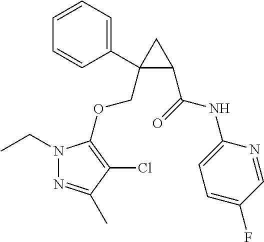 MS [M + H]⁺ = 429 |
| 75 | 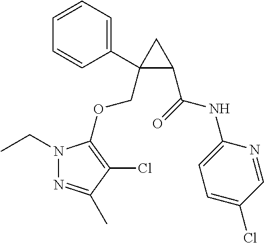 MS [M + H]⁺ = 445 |

The compounds of Examples 76 to 78 were each synthesized in the same manner as above.

TABLE 16

| Example | Structural formula, MS |
|---|---|
| 76 | 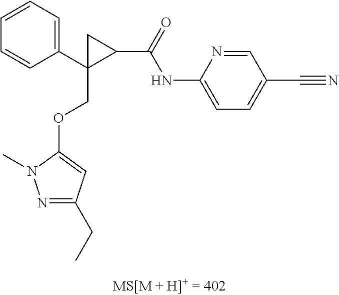 |
| 77 | 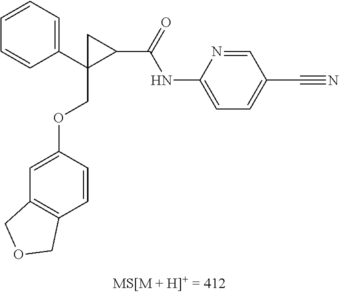 |
| 78 | 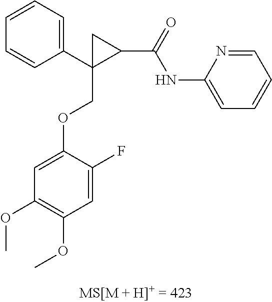 |

### Example 79

### Synthesis of N-(5-fluoropyridin-2-yl)-2-[(1-ethyl-3-methyl-1H-pyrazol-4-yl)oxymethyl]-2-phenylcyclopropanecarboxamide (79)

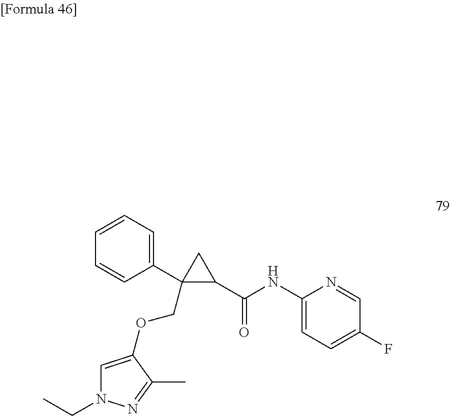

Trichloromethyl chloroformate (44 μl) was dissolved in THF (3 ml) and a THF solution (2 ml) of carboxylic acid Prep 19 (100 mg) and triethylamine (154 μl) were added dropwise while stirring under cooling on ice. After the reaction solution had been stirred for 30 minutes, a THF solution (2 ml) of 2-amino-5-fluoropyridine (123 mg) was added and the obtained mixture was stirred at room temperature for 20 hours. To the reaction solution, water was added and the obtained mixture was extracted with ethyl acetate. The organic layer was successively washed with a 1 N-sodium hydroxide solution, water and a saturated saline and dried over anhydrous magnesium sulfate. After filtration, the filtrate was concentrated under reduced pressure and the residue was purified by silica gel column chromatography (n-heptane:ethyl acetate). To the solid obtained, ether was added and filtration was performed to obtain the title compound (18 mg).

¹H-NMR (400 MHz, CDCl₃) δ (ppm): 1.33 (t, J=7.2 Hz, 3H), 1.57 (dd, J=8.0, 5.2 Hz, 1H), 1.84 (dd, J=5.6, 5.2 Hz, 1H), 1.91 (s, 3H), 2.07 (dd, J=8.0, 5.6 Hz, 1H), 3.90 (q, J=7.2 Hz, 2H), 4.14 (d, J=9.6 Hz, 1H), 4.28 (d, J=9.6 Hz, 1H), 6.85 (s, 1H), 7.24-7.48 (m, 6H), 8.12 (d, J=3.2 Hz, 1H), 8.16 (dd, J=9.2, 4.0 Hz, 1H), 8.29 (s, 1H).

MS [M+H]⁺=395

The compounds of Examples 80 to 84 were each synthesized by condensing carboxylic acid Preps 20 to 23 with an amine according to the process of Example 67.

TABLE 17

| Example | Structural formula | NMR and/or MS |
|---|---|---|
| 80 | 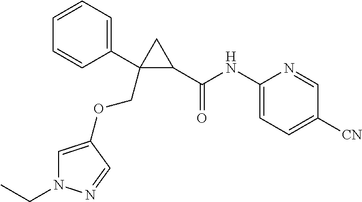 | ¹H-NMR (400 MHz, CDCl₃)δ(ppm): 1.36(t, J = 7.2 Hz, 3H), 1.62(dd, J = 8.0, 5.2 Hz, 1H), 1.88(dd, J = 6.0, 5.2 Hz, 1H), 2.10(dd, J = 8.0, 6.0 Hz, 1H), 3.96(q, J = 7.2 Hz, 2H), 4.18(d, J = 10.0 Hz, 1H), 4.37(d, J = 10.0 Hz, 1H), 6.90(d, J = 0.8 Hz, 1H), 7.02(d, J = 0.8 Hz, 1H), 7.25-7.31(m, 1H), 7.33- 7.38(m, 2H), 7.40-7.44(m, 2H), 7.90(dd, J = 8.8, 2.4 Hz, 1H), 8.28(dd, J = 8.8, 0.8 Hz, 1H), 8.47(s, 1H), 8.55(dd, J = 2.4, 0.8 Hz, 1H). MS[M + H]⁺ = 388 |
| 81 | 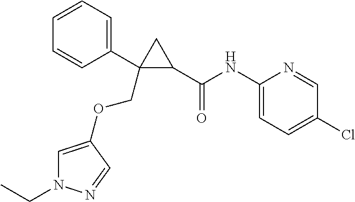 | ¹H-NMR (400 MHz, CDCl₃)δ(ppm): 1.35(t, J = 7.2 Hz, 3H), 1.57(dd, J = 8.0, 4.8 Hz, 1H), 1.85(dd, J = 5.6, 4.8 Hz, 1H), 2.07(dd, J = 8.0, 5.6 Hz, 1H), 3.96(q, J = 7.2 Hz, 2H), 4.20(d, J = 10.0 Hz, 1H), 4.36(d, J = 10.0 Hz, 1H), 6.90(d, J = 0.8 Hz, 1H), 7.04(d, J = 0.8 Hz, 1H), 7.25-7.30(m, 1H), 7.32- 7.38(m, 2H), 7.40-7.45(m, 2H), 7.63(ddd, J = 8.8, 2.8, 0.4 Hz, 1H), 8.13(d, J = 8.8 Hz, 1H), 8.22(dd, J = 2.8, 0.4 Hz, 1H), 8.28(s, 1H). MS[M + H]⁺ = 397 |
| 82 | 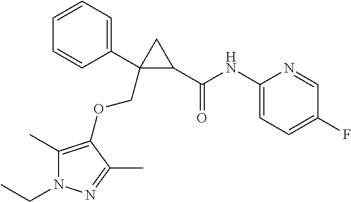 | ¹H-NMR (400 MHz, CDCl₃)δ(ppm): 1.24(t, J = 7.2 Hz, 3H), 1.50(dd, J = 8.0, 5.2 Hz, 1H), 1.79(dd, J = 6.0, 5.2 Hz, 1H), 1.81(s, 3H), 1.85(s, 3H), 2.11(dd, J = 8.0, 6.0 Hz, 1H), 3.82 (q, J = 7.2 Hz, 2H), 4.07(d, J = 9.6 Hz, 1H), 4.20(d, J = 9.6 Hz, 1H), 7.24- 7.40(m, 3H), 7.45(ddd, J = 9.2, 7.6, 2.8 Hz, 1H), 7.49-7.54(m, 2H), 8.13(d, J = 2.8 Hz, 1H), 8.29(dd, J = 9.2, 4.0 Hz, 1H), 8.33(s, 1H). MS[M + H]⁺ = 409 |
| 83 | 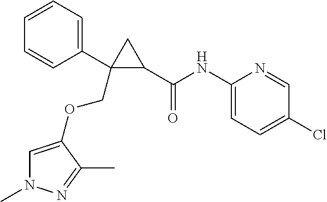 | ¹H-NMR (400 MHz, CDCl₃)δ(ppm): 1.57(dd, J = 8.4, 5.2 Hz, 1H), 1.90(s, 3H), 1.84(dd, J = 6.0, 5.2 Hz, 1H), 2.07(dd, J = 8.4, 6.0 Hz, 1H), 3.64(s, 3H), 4.12(d, J = 9.6 Hz, 1H), 4.28(d, J = 9.6 Hz, 1H), 6.81(s, 1H), 7.25-7.31(m, 1H), 7.23-7.38(m, 2H), 7.42-7.46(m, 2H), 7.64(dd, J = 8.8, 2.8 Hz, 1H), 8.12(d, J = 8.8 Hz, 1H), 8.22(dd, J = 2.8, 0.8 Hz, 1H), 8.28(s, 1H). MS[M + H]⁺ = 397 |
| 84 | 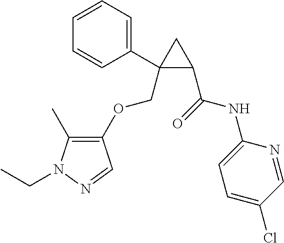 | MS[M + H]⁺ = 411 |

The compounds of Examples 85 to 89 were each synthesized by condensing carboxylic acid Prep 16 with an amine according to the process of Example 5.

TABLE 18

| Example | Structural formula | NMR and/or MS |
|---|---|---|
| 85 | 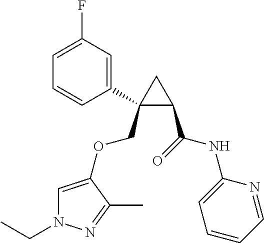 | ¹H-NMR (400 MHz, CDCl₃)δ(ppm): 1.34(t, J = 7.2 Hz, 3H), 1.54-1.58(m, 1H), 1.86(t, J = 5.6 Hz, 1H), 1.92(s, 3H), 2.05- 2.08(m, 1H), 3.90(q, J = 7.2 Hz, 2H), 4.15(d, J = 10.0 Hz, 1H), 4.27(d, J = 10.0 Hz, 1H), 6.88(s, 1H), 6.98(ddt, J = 8.0, 2.4, 1.2 Hz, 1H), 7.03(ddd, J = 7.2, 4.8, 1.2 Hz, 1H), 7.18(ddd, J = 10.0, 2.4, 1.2 Hz, 1H), 7.23(ddd, J = 8.0, 2.4, 1.2 Hz, 1H), 7.31(dt, J = 8.0, 6.0 Hz, 1H), 7.65-7.70(m, 1H), 8.12(brd, J = 8.0 Hz, 1H), 8.26-8.28(m, 2H). MS[M + H]⁺ = 395 |
| 86 | 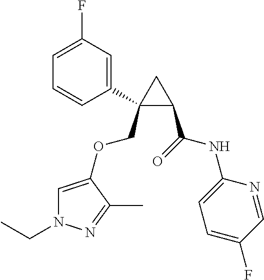 | ¹H-NMR (400 MHz, CDCl₃)δ(ppm): 1.34(t, J = 7.2 Hz, 3H), 1.57(dd, J = 8.0, 4.8 Hz, 1H), 1.86(dd, J = 6.0, 4.8 Hz, 1H), 1.93(s, 3H), 2.05(dd, J = 8.0, 6.0 Hz, 1H), 3.91(q, J = 7.2 Hz, 2H), 4.14(d, J = 10.0 Hz, 1H), 4.27(d, J = 10.0 Hz, 1H), 6.88(s, 1H), 6.98(ddt, J = 8.0, 2.4, 1.2 Hz, 1H), 7.17(ddd, J = 10.0, 2.4, 1.6 Hz, 1H), 7.22(ddd, J = 8.0, 1.6, 1.2 Hz, 1H), 7.32(dt, J = 8.0, 6.4 Hz, 1H), 7.41(ddd, J = 9.2, 4.0, 3.2 Hz, 1H), 8.13(d, J = 3.2 Hz, 1H), 8.14(dd, J = 9.2, 4.0 Hz, 1H), 8.23(s, 1H). MS[M + H]⁺ = 413 |
| 87 | 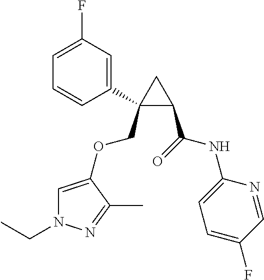 | ¹H-NMR (400 MHz, CDCl₃)δ(ppm): 1.35(t, J = 7.2 Hz, 3H), 1.56-1.60(m, 1H), 1.86(t, J = 5.2 Hz, 1H), 1.98(s, 3H), 2.07(dd, J = 8.0, 5.2 Hz, 1H), 3.92(q, J = 7.2 Hz, 2H), 4.14(d, J = 10.0 Hz, 1H), 4.32(d, J = 10.0 Hz, 1H), 6.88-6.91(m, 2H), 6.99(ddt, J = 8.0, 2.8, 0.8 Hz, 1H), 7.15-7.21(m, 2H), 7.31(dt, J = 8.0, 6.4 Hz, 1H), 7.72(brs, 1H), 8.10(ddd, J = 9.2, 7.2, 2.8 Hz, 1H), 8.19(s, 1H). MS[M + H]⁺ = 413 |
| 88 | 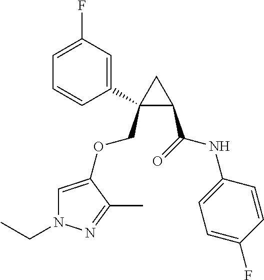 | ¹H-NMR (400 MHz, CDCl₃)δ(ppm): 1.34(t, J = 7.2 Hz, 3H), 1.54(dd, J = 8.0, 5.6 Hz, 1H), 1.84(t, J = 5.6 Hz, 1H), 1.98(s, 3H), 2.03(dd, J = 8.0, 5.6 Hz, 1H), 3.91(q, J = 7.2 Hz, 2H), 4.15(d, J = 10.0 Hz, 1H), 4.32(d, J = 10.0 Hz, 1H), 6.89(s, 1H), 6.95-7.02(m, 3H), 7.15-7.21(m, 2H), 7.28-7.34(m, 1H), 7.41- 7.44(m, 2H), 7.61(brs, 1H). MS[M + H]⁺ = 412 |
| 89 | 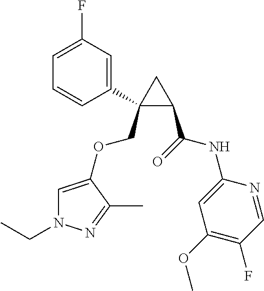 | ¹H-NMR (400 MHz, CDCl₃)δ(ppm): 1.34(t, J = 7.2 Hz, 3H), 1.57(dd, J = 8.0, 5.6 Hz, 1H), 1.84(t, J = 5.6 Hz, 1H), 1.94(s, 3H), 2.04(dd, J = 8.0, 5.6 Hz, 1H), 3.89-3.94(m, 5H), 4.14(d, J = 9.6 Hz, 1H), 4.28(d, J = 9.6 Hz, 1H), 6.88(s, 1H), 6.98(ddt, J = 8.0, 2.8, 1.2 Hz, 1H), 7.16(ddd, J = 10.0, 2.8, 1.6 Hz, 1H), 7.21(ddd, J = 8.0, 1.6, 1.2 Hz, 1H), 7.31(dt, J = 8.0, 6.0 Hz, 1H), 7.89(d, J = 7.2 Hz, 1H), 7.97(d, J = 3.2 Hz, 1H), 8.31(s, 1H). MS[M + H]⁺ = 443 |

The compounds of Examples 90 and 91 were each synthesized by condensing carboxylic acid Prep 16 with an amine according to the process of Example 68.

TABLE 19

| Example | Structural formula | NMR and/or MS |
|---|---|---|
| 90 | 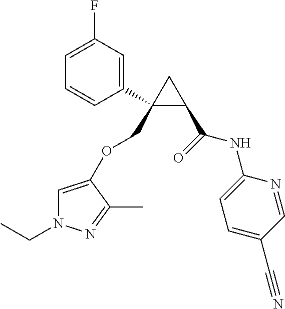 | ¹H-NMR (400 MHz, CDCl₃)δ(ppm): 1.35(t, J = 7.2 Hz, 3H), 1.62(dd, J = 8.0, 5.6 Hz, 1H), 1.89(t, J = 5.6 Hz, 1H), 1.91(s, 3H), 2.10(dd, J = 8.0, 5.6 Hz, 1H), 3.92(q, J = 7.2 Hz, 2H), 4.11(d, J = 10.0 Hz, 1H), 4.28(d, J = 10.0 Hz, 1H), 6.87(s, 1H), 7.00(ddt, J = 8.0, 2.4, 1.2 Hz, 1H), 7.17(ddd, J = 10.0, 2.4, 1.6 Hz, 1H), 7.22(ddd, J = 8.0, 1.6, 1.2 Hz, 1H), 7.32(dt, J = 8.0, 6.0 Hz, 1H), 7.91(ddd, J = 8.4, 2.4, 0.8 Hz, 1H), 8.27(dd, J = 8.4, 0.8 Hz, 1H), 8.47(s, 1H), 8.56(dd, J = 2.4, 0.8 Hz, 1H). MS[M + H]⁺ = 420 |
| 91 | 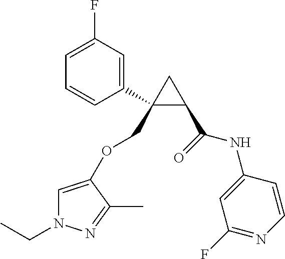 | ¹H-NMR (400 MHz, CDCl₃)δ(ppm): 1.35(t, J = 7.2 Hz, 3H), 1.60(dd, J = 8.0, 5.6 Hz, 1H), 1.88(t, J = 5.6 Hz, 1H), 1.95(s, 3H), 2.07(dd, J = 8.0, 5.6 Hz, 1H), 3.92(q, J = 7.2 Hz, 2H), 4.11(d, J = 10.0 Hz, 1H), 4.32(d, J = 10.0 Hz, 1H), 6.90(s, 1H), 6.98(ddt, J = 8.4, 2.8, 0.8 Hz, 1H), 7.13-7.19(m, 3H), 7.28- 7.34(m, 2H), 8.07(d, J = 5.6 Hz, 1H), 8.15(s, 1H). MS[M + H]⁺ = 413 |

### Example 92

### Synthesis of (1R,2S)-2-[(1,3-dimethyl-6-oxo-1,6-dihydropyridazin-4-yl)oxymethyl]-N-(5-methoxypyridin-3-yl)-2-phenylcyclopropanecarboxamide (92)

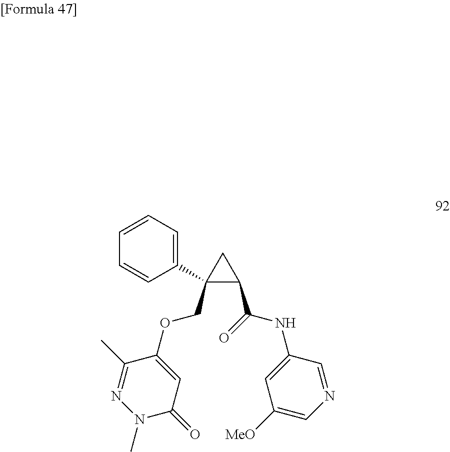

The carboxylic acid Prep 29 (15 mg), 5-methoxy-pyridin-3-ylamine (6.5 mg) and HATU (20 mg) were dissolved in NMP (0.75 ml) and N,N-diisopropylethylamine (12.6 μl) was added and the obtained mixture was stirred at room temperature for 20 hours. To the reaction solution, water was added and the obtained mixture was extracted with ethyl acetate. The organic layer was successively washed with water and a saturated saline, and dried over anhydrous magnesium sulfate. The solvent was distilled away under reduced pressure and the residue was purified by NH-silica gel column chromatography (n-heptane: ethyl acetate) to obtain the title compound (16.5 mg).

¹H-NMR (400 MHz, CDCl₃) δ (ppm): 1.64 (dd, J=8.0, 5.6 Hz, 1H), 1.89 (t, J=5.2 Hz, 1H), 2.06 (s, 3H), 2.16 (dd, J=8.0, 5.6 Hz, 1H), 3.60 (s, 3H), 3.82 (s, 3H), 4.35 (d, J=10.0 Hz, 1H), 4.42 (d, J=10.0 Hz, 1H), 6.01 (s, 1H), 7.27 (t, J=7.2 Hz, 1H), 7.34 (t, J=7.2 Hz, 2H), 7.41 (d, J=7.2 Hz, 2H), 7.91 (s, 1H), 8.05 (t, J=2.0 Hz, 1H), 8.07 (s, 1H), 8.32 (brs, 1H).

### Example 93

### Synthesis of (1R,2S)-2-[(1,3-dimethyl-6-oxo-1,6-dihydropyridazin-4-yl)oxymethyl]-N-(4-fluorophenyl)-2-phenylcyclopropanecarboxamide (93)

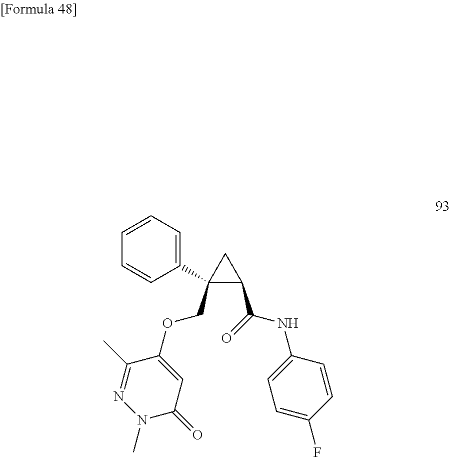

The compound was synthesized from carboxylic acid Prep 29 by the same manner as in Example 92.

MS [M+H]⁺=408

The compounds of Examples 94 and 95 were each synthesized by condensing carboxylic acid Prep 29 with an amine according to the process of Example 92.

TABLE 20

| Example | Structural formula, MS |
|---|---|
| 94 | 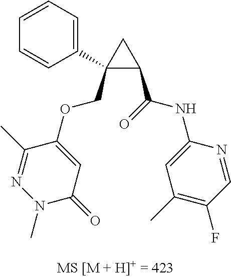 |
| 95 | 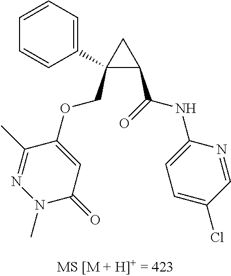 |

The compounds of Examples 96 to 98 were each synthesized by condensing carboxylic acid Prep 30 with an amine according to the process of Example 5.

TABLE 21

| Example | Structural formula, MS |
|---|---|
| 96 | 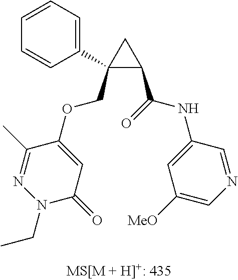 |
| 97 | 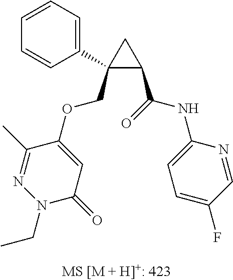 |
| 98 | 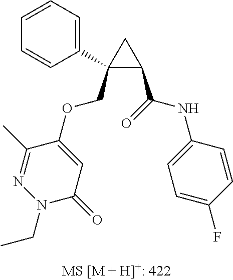 |

### Example 99

### Synthesis of N-(pyridin-2-yl)-2-[2-(3,4-dimethoxyphenyl)ethyl]-2-phenylcyclopropanecarboxamide (99)

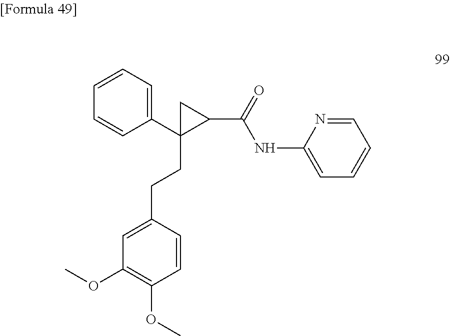

To an acetone (1.5 ml)-water (0.5 ml) aqueous solution of aldehyde Prep 32 (36.6 mg), 2-methyl-2-butene (62.5 μl), sodium dihydrogen phosphate monohydrate (14.2 mg) and sodium hypochlorite (32 mg) were added and the obtained mixture was stirred at room temperature for 4 hours. To the reaction mixture, 1 N hydrochloric acid was added and the obtained mixture was subjected to liquid separation and extraction with ethyl acetate. The organic layer was dried over anhydrous magnesium sulfate and filtered. The filtrate was concentrated under reduced pressure. To a DMF solution (2 ml) of the obtained residue, 2-aminopyridine (44.4 mg), HATU (89.7 mg) and N,N-diisopropylethylamine (102 μl) were added and the obtained mixture was stirred at room temperature for 2 days. To the reaction mixture, a saturated sodium bicarbonate aqueous solution was added and the obtained mixture was subjected to liquid separation and extraction with ethyl acetate. The organic layer was dried over anhydrous magnesium sulfate and filtered. The filtrate was concentrated under reduced pressure and purified by NH-silica gel column chromatography (n-heptane:ethyl acetate=19:1→1:2) and fractionated by LC-MS. The mass fraction of the desired substance was concentrated and the obtained residue was again purified by NH-silica gel column chromatography (n-heptane:ethyl acetate=2:1→1:1) to obtain the title compound (6.3 mg).

¹H-NMR (400 MHz, CDCl₃) δ (ppm): 1.45 (dd, J=8.0, 4.8 Hz, 1H), 1.59-1.61 (m, 1H), 1.93 (dd, J=7.6, 5.2 Hz, 1H), 2.17-2.21 (m, 2H), 2.43-2.49 (m, 2H), 3.79 (s, 3H), 3.79 (s, 3H), 6.55-6.59 (m, 2H), 6.68 (d, J=7.6 Hz, 1H), 7.02 (dd, J=8.0, 5.2 Hz, 1H), 7.28-7.37 (t, J=7.2 Hz, 2H), 7.41 (d, J=7.2 Hz, 2H), 7.91 (s, 1H), 8.05 (m, 5H), 7.68-7.72 (m, 1H), 8.24-8.30 (m, 3H).

### Example 100

### Synthesis of (1R,2R)—N-(5-cyanopyridin-2-yl)-2-[2-(2-ethyl-4-methyl-1,3-thiazol-5-yl)ethyl]-2-phenylcyclopropanecarboxamide (100)

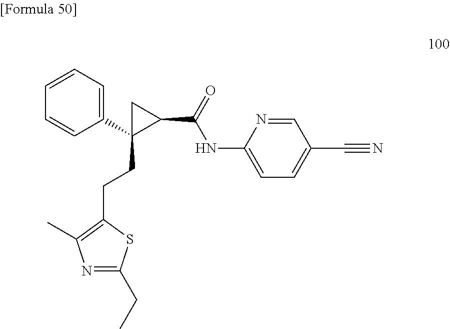

The compound was synthesized by amidating a carboxylic acid form, which was synthesized from compound Prep 33 by the same manner as in Production Examples 1-(7), (8), by the same manner as in Example 3 and subjecting the racemic form obtained to chiral resolution.

MS [M+H]⁺=417

The compounds of Examples 101 to 103 were each synthesized by condensing carboxylic acid Prep 31 with an amine according to the process of Example 68.

TABLE 22

| Example | Structural formula, MS or NMR | Example | Structural formula, MS or NMR |
|---|---|---|---|
| 101 | 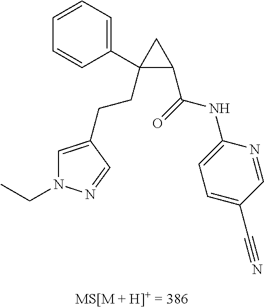 | 102 | 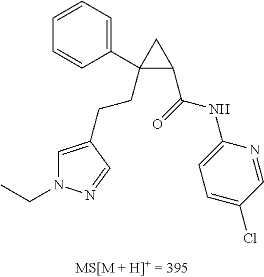 |

| | | |
|---|---|---|
| 103 | 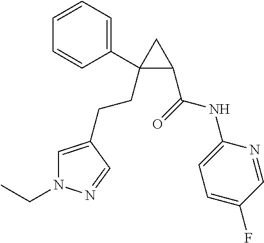 | ¹H-NMR (400 MHz, CDCl₃)δ(ppm): 1.24-1.30(m, 1H), 1.39(t, J = 7.4 Hz, 3H), 1.48(dd, J = 8.2, 6.0 Hz, 1H), 1.85- 1.92(m, 1H), 2.08-2.21(m, 2H), 2.26-2.45(m, 2H), 4.02(q, J = 7.4 Hz, 2H), 7.05(s, 1H), 7.18(s, 1H), 7.24-7.38(m, 5H), 7.42-7.49(m, 1H), 8.12(d, J = 2.8 Hz, 1H), 8.23- 8.34(m, 2H). |

The compounds of Examples 104 to 107 were each synthesized by condensing the corresponding carboxylic acid and 2-aminopyridine according to the method of Example 6.

TABLE 23

| Example | Structural formula, MS |
|---|---|
| 104 | 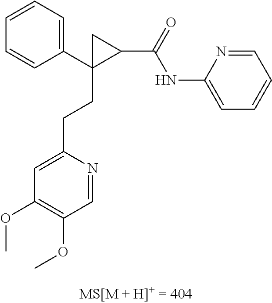 |
| 105 | 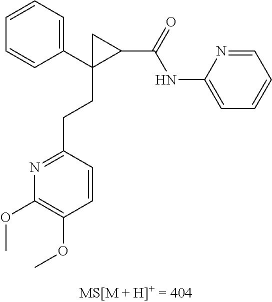 |
| 106 | 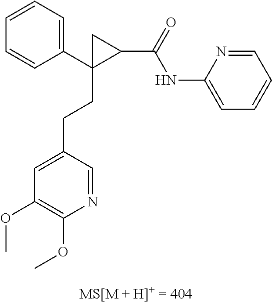 |
| 107 | 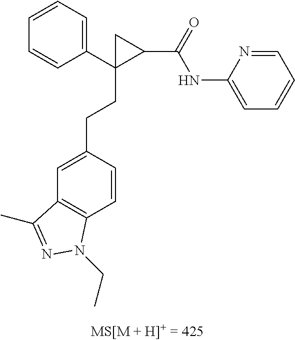 |

### Example 108

### Synthesis of (1R,2S)-2-[2-(3-difluoromethyl-5-methyl-1H-1,2,4-triazol-1-yl)ethyl]-N-(5-fluoro-4-methylpyridin-2-yl)-2-phenylcyclopropanecarboxamide (108)

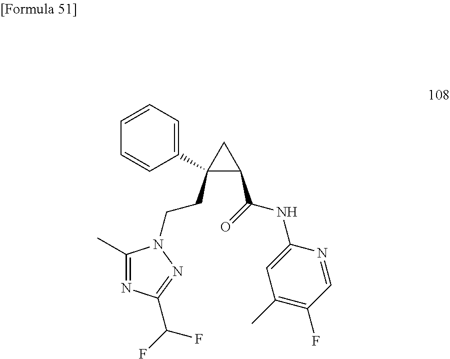

The carboxylic acid Prep 34 (1000 mg) was dissolved in THF (1.2 ml) and oxalyl chloride (16 μl) and DMF (several drops) were added and the obtained mixture was stirred at room temperature for 2 hours. The reaction solution was concentrated under reduced pressure to obtain a crude acid chloride. To a THF solution (1.2 ml) of 2-amino-5-fluoropyridine (31.4 mg), N,N-diisopropylethylamine (47.3 μl) was added and the temperature of the reaction solution was increased to 60° C. To this, a THF solution (100 ml) of the crude acid chloride was added dropwise and the obtained mixture was stirred for 1 hour while maintaining the temperature. The reaction mixture was allowed to cool to room temperature and the obtained mixture was stirred overnight. Then, to the reaction solution, a saturated sodium bicarbonate aqueous solution was added and the obtained mixture was extracted with ethyl acetate. The organic layer was dried over anhydrous magnesium sulfate and the filtrate was concentrated under reduced pressure. The residue was purified by silica gel column chromatography (ethyl acetate:methanol) to obtain the title compound (21.03 mg).

¹H-NMR (400 MHz, CDCl₃) δ (ppm): 1.49-1.55 (m, 2H), 1.89 (t, J=6.4 Hz, 1H), 2.29 (s, 3H), 2.33 (s, 3H), 2.49-2.54 (m, 2H), 3.95-4.03 (m, 2H), 6.55 (t, J=14.0 Hz, 1H), 7.26-7.38 (m, 5H), 7.97 (s, 1H), 8.10-8.12 (m, 1H), 8.60 (brs, 1H).

The compounds of Examples 109 to 112 were each synthesized by condensing carboxylic acid Prep 34 with an amine according to the process of Example 108.

TABLE 24

| Example | Structural formula, MS |
|---|---|
| 109 | 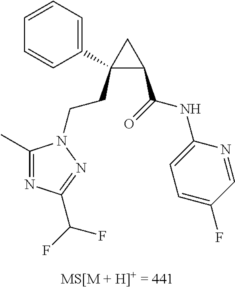 |
| 110 | 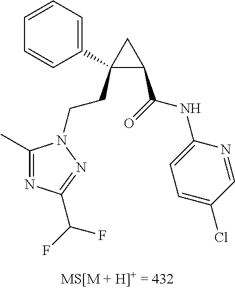 |
| 111 | 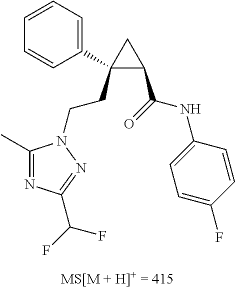 |
| 112 | 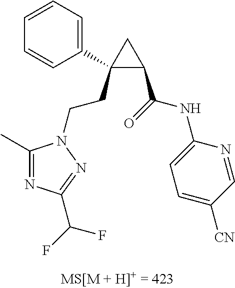 |

The compounds of Examples 113 and 114 were each synthesized by condensing carboxylic acid Prep 38 with an amine according to the process of Example 3.

TABLE 25

| Example | Structural formula, MS |
|---|---|
| 113 | 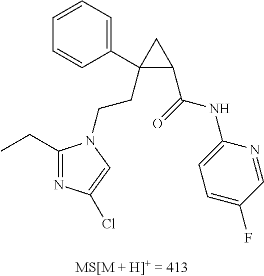 |
| 114 | 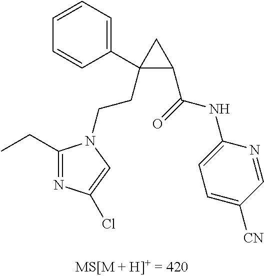 |

The compounds of Examples 115 and 116 were each synthesized by condensing carboxylic acid Prep 36 with an amine according to the process of Example 68.

TABLE 26

| Example | Structural formula, MS |
|---|---|
| 115 | 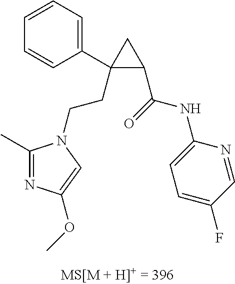 |
| 116 | 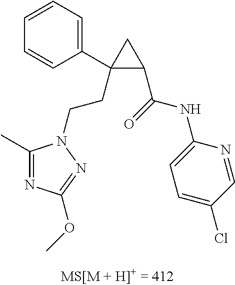 |

The compounds of Examples 117 and 118 were each synthesized by condensing carboxylic acid Prep 39 with an amine according to the process of Example 67.

TABLE 27

| Example | Structural formula, MS |
|---|---|
| 117 | 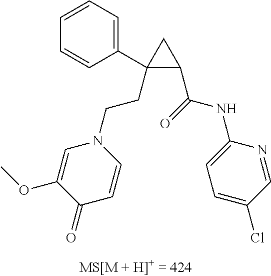 |
| 118 | 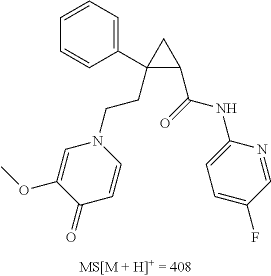 |

The compounds of Examples 119 and 120 were each synthesized by condensing carboxylic acid Prep 40 with an amine according to the process of Example 68.

TABLE 28

| Example | Structural formula, MS |
|---|---|
| 119 | 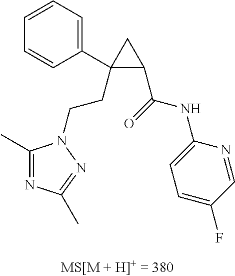 |
| 120 | 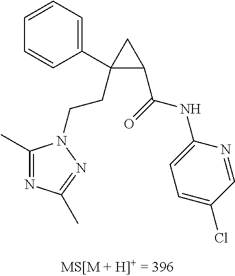 |

The compounds of Examples 121 to 124 were each synthesized by condensing carboxylic acid Prep 41 with an amine according to the process of Example 1.

TABLE 29

| Example | Structural formula, MS |
|---|---|
| 121 | 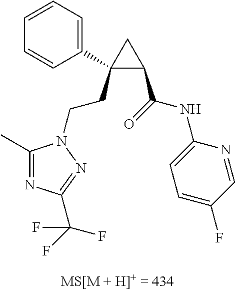 |
| 122 | 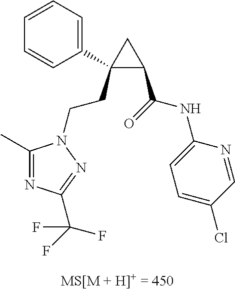 |
| 123 | 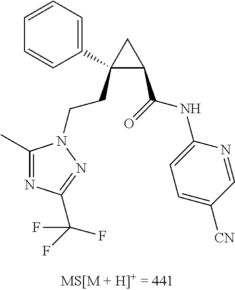 |
| 124 | 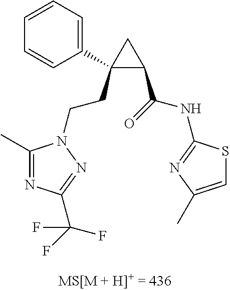 |

The compounds of Examples 125 to 127 were each synthesized by condensing carboxylic acid Prep 42 with an amine according to the process of Example 68.

TABLE 30

| Example | Structural formula, MS |
|---|---|
| 125 | 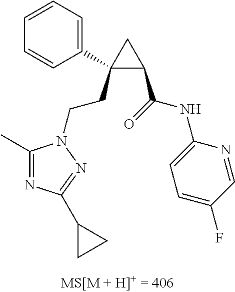 |
| 126 | 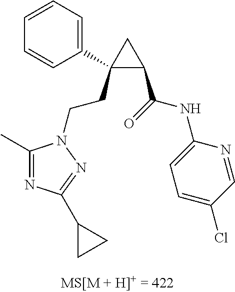 |
| 127 | 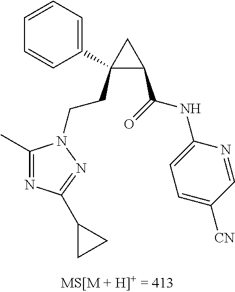 |

The compounds of Examples 128 and 129 were each synthesized by condensing carboxylic acid Prep 43 with an amine according to the process of Example 1.

TABLE 31

| Example | Structural formula, MS |
|---|---|
| 128 | 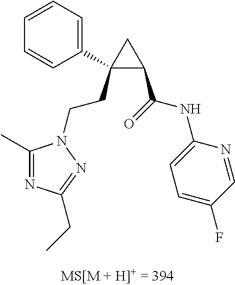 |
| 129 | 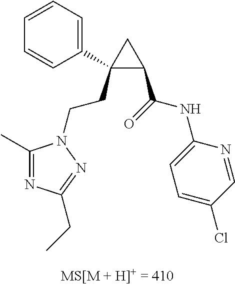 |

### Example 130

### Synthesis of N-(5-fluoropyridin-2-yl)-2-[2-(2-ethyl-4-methyl-1H-imidazol-1-yl)-ethyl]-2-phenylcyclopropanecarboxamide (130)

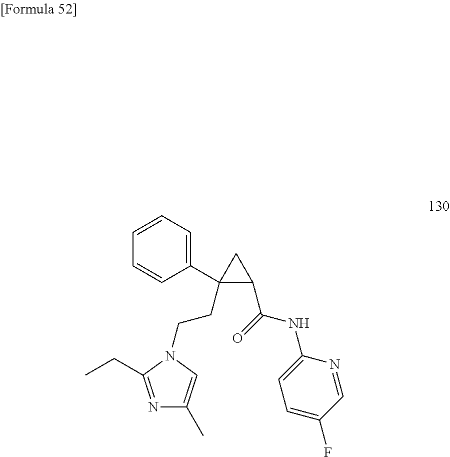

The title compound was obtained by amidating carboxylic acid Prep 44 according to the method of Example 68.

MS [M+H]⁺=393

### Example 131

### Synthesis of (1R,2S)-2-[2-(5-ethyl-3-methyl-1H-1,2,4-triazol-1-yl)ethyl]-N-(5-fluoropyridin-2-yl)-2-phenylcyclopropanecarboxamide (131)

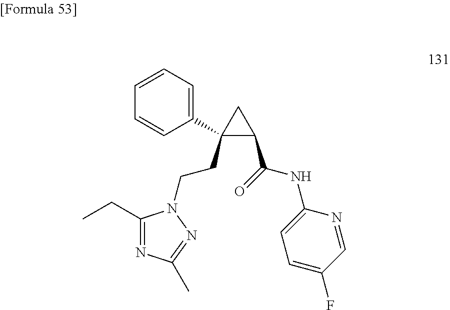

The title compound was obtained by amidating carboxylic acid Prep 45 according to the method of Example 68.

MS [M+H]⁺=394

### Example 132

### Synthesis of N-(5-chloropyridin-2-yl)-2-[2-(5-methyl-1,3,4-oxadiazol-2-yl)ethyl]-2-phenylcyclopropanecarboxamide trifluoroacetate (132)

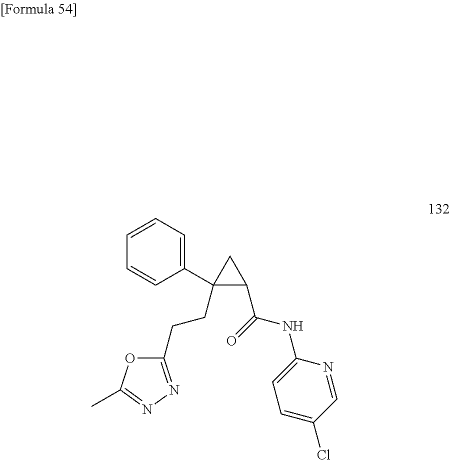

The title compound was obtained by amidating carboxylic acid Prep 46 according to the method of Example 68.

MS [M+H]⁺=383

### Example 133

### Synthesis of N-(5-fluoropyridin-2-yl)-2-[(3,4-dimethoxyphenyl)sulfonylmethyl]-2-phenylcyclopropanecarboxamide (133)

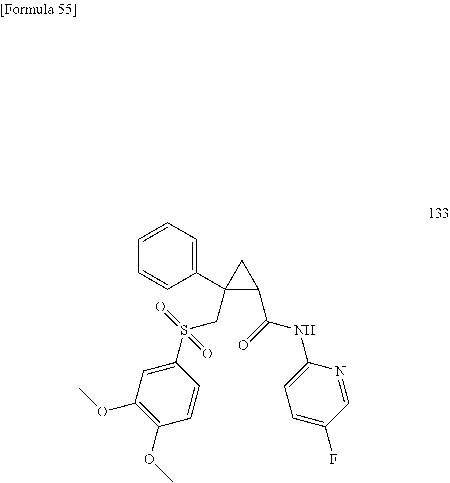

To a THF solution (2 ml) of trichloromethyl chloroformate (20.1 μl), carboxylic acid Prep 47 (50 mg) was added. To the reaction solution, a THF solution (1 ml) of triethylamine (69.4 μl) was added dropwise and the obtained mixture was stirred at room temperature for 30 minutes. Thereafter, 2-amino-5-fluoropyridine (98.9 mg) was added and the obtained mixture was stirred at room temperature for 5 hours. To the reaction solution, ethyl acetate was added and washed with a saturated ammonium chloride aqueous solution, a saturated sodium bicarbonate aqueous solution, and a saturated saline. The organic layer was dried over anhydrous magnesium sulfate and filtered. The filtrate was concentrated under reduced pressure and the crude product obtained was purified by LC-MS to obtain the title compound (25.52 mg).

¹H-NMR (400 MHz, CDCl₃) δ (ppm): 1.39 (dd, J=8.2, 4.4 Hz, 1H), 1.72 (t, J=5.2 Hz, 1H), 2.66-2.71 (m, 1H), 3.77 (s, 3H), 3.87 (s, 3H), 3.89 (d, J=15.2 Hz), 4.03 (d, J=15.2 Hz), 6.72-6.75 (m, 1H), 6.93-6.94 (m, 1H), 7.13-7.37 (m, 5H), 7.89 (m, 1H), 8.13 (brs, 1H), 8.50-8.80 (m, 2H).

### Example 134

### Synthesis of N-(5-chloropyridin-2-yl)-2-[(3,4-dimethoxyphenyl)sulfonylmethyl]-2-phenylcyclopropanecarboxamide (134)

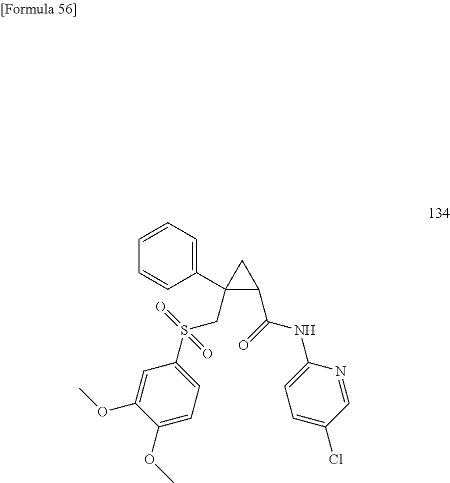

The compound was synthesized according to the method of Example 133.

MS [M+H]⁺=487

### Test Example

### 1. Measurement of Orexin-Receptor Binding Ability

The assay was carried out using a 96-well Wheat Germ Agglutinin Flash Plate (PerkinElmer). The volume in a single assay well was 100 ul, and the composition of the reaction solution was as follows:

25 mM HEPES (pH 7.5), 1 mM CaCl₂, 4.5 mM MgCl₂, 0.5% BSA (bovine serum albumin), 0.1% sodium azide, 0.05% Tween-20, and 0.2% DMSO.

Cell membranes were prepared from recombinant CHO cells that expressed OX2 or OX1. The cell membranes were used in an amount of 5 μg protein/assay. Test compound in various concentrations, and 0.2 nM [¹²⁵I]-OX-A as tracer were added to the cell membranes, and then allowed to react at room temperature for 30 minutes. After completion of the reaction, the reaction solution as a whole was discarded, and the wells were then washed once with 200 ul of wash buffer (25 mM HEPES (pH 7.5), 1 mM CaCl₂, 5 mM MgCl₂, 0.5% BSA, 0.1% sodium azide, 0.05% Tween-20, and 525 mM sodium chloride). Finally, the radioactivity of each well was measured using a scintillation counter (TopCount, PerkinElmer). The obtained results are shown in terms of IC50 values (nM) in the following table.

TABLE 32

| Example | OX1 | OX2 |
|---|---|---|
| No. | (IC50, nM) | (IC50, nM) |
| 1 | 7 | 7 |
| 4 | 129 | 13 |
| 6 | 19 | 3 |
| 7 | 1901 | 43 |
| 8 | 1259 | 17 |
| 10 | 56 | 3 |
| 11 | 333 | 29 |
| 14 | 438 | 46 |
| 18 | 105 | 35 |
| 21 | 505 | 28 |
| 22 | 296 | 35 |
| 23 | 847 | 14 |
| 24 | 805 | 48 |
| 25 | 369 | 27 |
| 26 | 27 | 43 |
| 27 | 37 | 4 |
| 28 | 38 | 25 |
| 34 | 20 | 6 |
| 35 | 2041 | 47 |
| 36 | 283 | 22 |
| 37 | 403 | 18 |
| 38 | 496 | 31 |
| 40 | 1047 | 36 |
| 41 | 374 | 35 |
| 54 | 61 | 3 |
| 55 | 675 | 33 |
| 56 | 160 | 14 |
| 57 | 483 | 46 |
| 58 | 467 | 45 |
| 61 | 520 | 70 |
| 62 | 44 | 61 |
| 65 | 57 | 26 |
| 66 | 74 | 21 |
| 67 | 33 | 7 |
| 68 | 174 | 8 |
| 69 | 2126 | 69 |
| 71 | 1313 | 45 |
| 74 | 359 | 30 |
| 75 | 477 | 26 |
| 78 | 212 | 20 |
| 79 | 213 | 54 |
| 80 | 359 | 54 |
| 81 | 163 | 33 |
| 83 | 682 | 15 |
| 85 | 59 | 8 |
| 86 | 15 | 5 |
| 87 | 136 | 21 |
| 88 | 84 | 12 |
| 89 | >200 | 93 |
| 90 | 13 | 8 |
| 91 | >200 | 93 |
| 92 | 1357 | 5 |
| 93 | 410 | 27 |
| 94 | 771 | 39 |
| 95 | 1596 | 59 |
| 98 | 132 | 55 |
| 99 | 632 | 43 |
| 100 | 58 | 9 |
| 102 | 364 | 22 |
| 108 | 101 | 6 |
| 109 | 1330 | 71 |
| 110 | 196 | 36 |
| 111 | 655 | 41 |
| 112 | — | 33 |
| 113 | 944 | 49 |
| 114 | 868 | 49 |
| 116 | >2000 | 89 |
| 117 | 456 | 46 |
| 121 | 166 | 19 |
| 122 | 98 | 3 |
| 124 | 2252 | 55 |
| 127 | 266 | 23 |
| 134 | 261 | 79 |

### 2. Measurement of Antagonism (PLAP Assay)

The antagonistic function of the compound of the present invention to prevent the activation of OX2 and OX1 by orexin-A (OX-A), which is a natural peptide agonist, was measured using a cell-based reporter assay. A HEK-293 cell line expressing genetically recombinant human OX2 (accession No. NM_{—}001526.3) or a HEK-293 cell line expressing genetically recombinant human OX1 (accession No. NM_{—}001525.2), which had pBabeCLIH as expression vector, was used. The cells were plated at a density of 10,000 cells/well onto a non-coated 96-well plate in Dulbecco's modified Eagle medium (Sigma Cat No. D6046: 10% v/v heat-inactivated fetal bovine serum was contained). The cells were cultured at 37° C. overnight, so that they could adhere to the plate. On the following day, cells were incubated with a compound of the present invention dissolved in Dulbecco's modified Eagle medium (Sigma Cat No. A8806: 0.1% w/v bovine serum albumin was contained), and added to the cell plate to reach a final concentration of 0.1% dimethyl sulfoxide.

The thus obtained mixture was incubated at room temperature for 1 hour. Thereafter, human OX-A and forskolin were dissolved in the same medium as described above, which contained fetal bovine serum albumin, and the medium was then added to the cells, resulting in a final concentration of 300 nM forskolin. Subsequently, the cells were cultured at 37° C. for approximately 18 to 24 hours. During the culture, as a result of activation of the orexin receptor and subsequent dose-dependent increase in intracellular calcium concentration, a reporter enzyme, placental alkaline phosphatase (PLAP), was expressed under the control of a CRE x4+VIP promoter in a pBabeCLcre4vPdNN vector and secreted into the culture medium supernatant. On the following day, reporter enzyme activity was detected by mixing 5 ul of the culture medium supernatant with 20 ul of detection buffer (containing 1.34 g/L sodium bicarbonate, 1.27 g/L sodium carbonate and 0.2 g/L magnesium sulfate heptahydrate in water) and 25 ul of Lumi-Phos530 reagent (Wako Pure Chemical Industries Ltd.), followed by incubating the obtained mixture light-protected at room temperature for 2 hours, before performing luminescence measurement (ARVO Reader, PerkinElmer). The Kd value of human OX-A with respect to each receptor was measured by titration from 0 to 300 nM. Then, the IC50 value of the compound of the present invention with respect to the activity of 1 nM human OX-A was converted into a Ki value (nM) using the Cheng-Prusoff equation. The obtained Ki values (nM) are shown in the following table.

TABLE 33

| Example | OX1 PLAP | OX2 PLAP |
|---|---|---|
| No. | (Ki, nM) | (Ki, nM) |
| 1 | 395 | 54 |
| 4 | 65.2 | 5.1 |
| 6 | 9.8 | 0.8 |
| 7 | >816 | 55.9 |
| 8 | >816 | 92.1 |
| 10 | 68.6 | 4.2 |
| 11 | 572 | 48.2 |
| 14 | >816 | 99 |
| 18 | 186 | 23.7 |
| 21 | >816 | 77.1 |
| 22 | >816 | >614 |
| 23 | 624 | 15.8 |
| 24 | >816 | 83.5 |
| 25 | >816 | 391 |
| 26 | 70 | 12.8 |
| 27 | 98 | 3.4 |
| 28 | 73.8 | 11 |
| 34 | 24 | 4.5 |
| 35 | 1900 | 122 |
| 36 | 699 | 43 |
| 37 | 593 | 14 |
| 38 | 765 | 52.5 |
| 40 | 752 | 51.5 |
| 41 | 297 | 24.8 |
| 54 | 37.5 | 0.8 |
| 55 | >467 | 43.6 |
| 56 | 158 | 9.1 |
| 57 | >467 | 31.7 |
| 58 | >467 | 20.9 |
| 61 | 275.5 | 32.9 |
| 62 | 481 | 30.2 |
| 68 | 261 | 19.4 |
| 74 | >467 | 30.7 |
| 75 | >816 | 30.8 |
| 78 | 342 | 19.2 |
| 79 | 275 | 10.5 |
| 80 | >467 | 190 |
| 81 | >467 | 78 |
| 99 | >816 | 154.5 |
| 100 | 47.5 | 5.2 |
| 113 | >467 | 62.7 |
| 114 | >467 | 210 |
| 121 | 406 | 31.4 |
| 122 | 140 | 10 |
| 134 | >467 | 268 |

### 3. Sleep Experiment

As a method for measuring the influence of the present compound on sleep time, electroencephalogram (EEG) and electromyogram (EMG) measurements were carried out in mice (C57BL/6NCrlCrLj).

In order to measure brain waves and muscle signals, EEG and EMG electrode implantation was performed on individual mice, and the mice were then housed in a state in which they could freely move and habituate in individual recording cages for 1 week or longer. Thereafter, amplified EEG and EMG signals were digitally recorded.

Mice received either oral administration of vehicle or test compound in vehicle, after which sleep/wake behavior of mice was recorded for 3 hours.

For sleep analysis, automatic analysis software from Kissei Comtec Co., Ltd. was used to analyze EEG frequency and EMG activity signals in detail and to determine sleep and wake states. Thereafter, accumulated sleeping time over 3 hours was calculated.

The effect of the compound to increase sleep time was evaluated as the difference between sleeping time on the vehicle-administration day and the sleep time on the subsequent drug-administration day. The obtained results are shown in the following table.

TABLE 34

| | Sleep extended time |
|---|---|
| Example No. | (min/3 hrs) |
| 4 | 57 |
| 68 | 12.4 |
| 121 | 21.3 |
| 122 | 21.8 |

As described in detail above, the cyclopropane compounds of the present invention, a pharmaceutically acceptable salt thereof or a solvate thereof has orexin receptor antagonism, promote sleep time increase, and therefore has the potential to be useful for the treatment of sleep disturbance, for example, insomnia, via orexin receptor antagonism.

1. A compound represented by the following formula (I) or a pharmaceutically acceptable salt thereof: 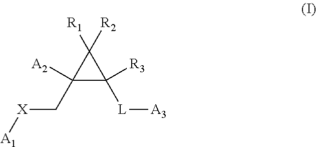
wherein
A₁ represents an aryl group selected from Group 1, which may optionally have 1 to 3 substituents selected from Substituent group α or a heteroaryl group selected from Group 2, which may optionally have 1 to 3 substituents selected from Substituent group α,
A₂ and A₃ each independently represent an aryl group selected from Group 1, which may optionally have 1 to 3 substituents selected from Substituent group α or a heterocyclyl group selected from Group 3, which may optionally have 1 to 3 substituents selected from Substituent group α,
R₁, R₂ and R₃ each independently represent a hydrogen atom, a halogen atom or a C₁₋₆ alkyl group which may optionally have 1 to 3 substituents selected from Substituent group β,
X represents an oxygen atom, a C₁₋₆ alkylene group, a formula —NR₄— (wherein R₄ represents a hydrogen atom or a C₁₋₆ alkyl group) or a sulfonyl group,
L represents a bond or a formula —CONR₅— (wherein R₅ represents a hydrogen atom or a C₁₋₆ alkyl group), wherein
Group 1: a phenyl group, a naphthyl group, an azulenyl group, an anthryl group and a phenanthryl group;
Group 2: a furyl group, a thienyl group, a pyrrolyl group, an imidazolyl group, a triazolyl group, a tetrazolyl group, a thiazolyl group, a pyrazolyl group, an oxazolyl group, an isoxazolyl group, an isothiazolyl group, a furazanyl group, a thiadiazolyl group, an oxadiazolyl group, a pyridyl group, a pyrazinyl group, a pyridazinyl group, a triazinyl group, an indolyl group, an isoindolyl group, an indazolyl group, a benzoxazolyl group, a benzisoxadiazolyl group, a benzothiazolyl group, a benzisothiazolyl group, a quinolyl group and an isoquinolyl group;
Group 3: a furyl group, a thienyl group, a pyrrolyl group, an imidazolyl group, a triazolyl group, a tetrazolyl group, a thiazolyl group, a pyrazolyl group, an oxazolyl group, an isoxazolyl group, an isothiazolyl group, a furazanyl group, a thiadiazolyl group, an oxadiazolyl group, a pyridyl group, a pyrazinyl group, a pyridazinyl group, a pyrimidinyl group, a triazinyl group, a 2-pyridonyl group, a 4-pyridonyl group, a pyridazidonyl group, a pyrimididonyl group, a purinyl group, a pteridinyl group, a quinolyl group, an isoquinolyl group, a naphthylidyl group, a quinoxalyl group, a cinnolyl group, a quinazolyl group, a phthalazyl group, an imidazopyridyl group, an imidazothiazolyl group, an imidazoxazolyl group, a benzimidazolyl group, an indolyl group, an isoindolyl group, an indazolyl group, a pyrrolopyridyl group, a thienopyridyl group, a fluoropyridyl group, a benzoxazolyl group, a benzisoxadiazolyl group, a benzothiazolyl group, a benzisothiazolyl group, a pyridopyrimidinyl group, a benzofuryl group, a benzothienyl group, a benzothiadiazolyl group, a benzo[1,3]dioxolyl group, a thienofuryl group, a dihydroisobenzofuranyl group, a chromanyl group, an isochromanyl group, a 1,3-dioxaindanyl group, a 1,4-dioxatetralinyl group and dihydrobenzo[1,4]oxazinyl group;
Substituent group α: a cyano group, a halogen atom, formula —NR₆R₇ (wherein R₆ and R₇ each independently represent a hydrogen atom or a C₁₋₆ alkyl group), a C₁₋₆ alkyl group which may optionally have 1 to 3 substituents selected from Substituent group β, a C₁₋₆ alkoxy group which may optionally have 1 to 3 substituents selected from Substituent group β, a C₁₋₆ alkylcarbonyl group which may optionally have 1 to 3 substituents selected from Substituent group β, a C₁₋₆ alkylsulfonyl group which may optionally have 1 to 3 substituents selected from Substituent group β, an aryl group selected from group 1, which may optionally have 1 to 3 substituents selected from Substituent group β, and a heterocyclyl group selected from group 3, which may optionally have 1 to 3 substituents selected from Substituent group β; and
Substituent group β: a cyano group, a halogen atom, a hydroxy group, a C₃₋₈ cycloalkyl group and a C₁₋₆ alkoxy group.

2. The compound according to claim 1, which is represented by the following formula (II), or a pharmaceutically acceptable salt thereof: 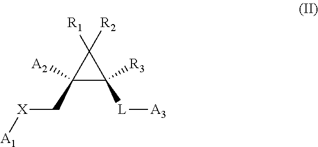
wherein A₁, A₂, A₃, R₁, R₂, R₃, X and L have the same definitions as those described in claim 1.

3. The compound according to claim 1, or a pharmaceutically acceptable salt thereof, wherein R₁, R₂ and R₃ each represent a hydrogen atom.

4. The compound according to claim 3, or a pharmaceutically acceptable salt thereof, wherein L represents a formula —CONH—.

5. The compound according to claim 4, or a pharmaceutically acceptable salt thereof, wherein X represents an oxygen atom.

6. The compound according to claim 4, or a pharmaceutically acceptable salt thereof, wherein X represents methylene.

7. The compound according to claim 5, or a pharmaceutically acceptable salt thereof, wherein A₂ and A₃ each independently represent an aryl group or a heterocyclyl group which may optionally have 1 to 3 substituents selected from a cyano group, a halogen atom, a C₁₋₆ alkyl group, a halo-C₁₋₆ alkyl group and a C₁₋₆ alkoxy group.

8. The compound according to claim 7, or a pharmaceutically acceptable salt thereof, wherein A₂ and A₃ each independently represent a phenyl group, a naphthyl group, a furyl group, a thienyl group, a pyrrolyl group, an imidazolyl group, a triazolyl group, a tetrazolyl group, a thiazolyl group, a pyrazolyl group, an oxazolyl group, an isoxazolyl group, an isothiazolyl group, a furazanyl group, a thiadiazolyl group, an oxadiazolyl group, a pyridyl group, a pyrazinyl group, a pyridazinyl group, a pyrimidinyl group, a triazinyl group, a quinolyl group or an isoquinolyl group, which may optionally have 1 to 3 substituents selected from a cyano group, a halogen atom, a C₁₋₆ alkyl group, a halo-C₁₋₆ alkyl group and C₁₋₆ alkoxy group.

9. The compound according to claim 8, or a pharmaceutically acceptable salt thereof, wherein A₂ represents a phenyl group which may optionally have 1 to 3 substituents selected from a cyano group, a halogen atom, a C₁₋₆ alkyl group, a halo-C₁₋₆ alkyl group and a C₁₋₆ alkoxy group.

10. The compound according to claim 9, or a pharmaceutically acceptable salt thereof, wherein A₃ represents a phenyl group or a pyridyl group which may optionally have 1 to 3 substituents selected from a cyano group, a halogen atom, a C₁₋₆ alkyl group, a halo-C₁₋₆ alkyl group and a C₁₋₆ alkoxy group.

11. The compound according to claim 10, or a pharmaceutically acceptable salt thereof, wherein A₁ represents a phenyl group, a pyrazolyl group or a triazolyl group which may optionally have 1 to 3 substituents selected from a halogen atom, a C₁₋₆ alkyl group, a halo-C₁₋₆ alkyl group, a C₁₋₆ alkoxy group, a C₃₋₈ cycloalkyl group and a C₁₋₆ alkoxy group.

12. A compound, which is selected from the following compounds:
1) N-phenyl-2-[(3,4-dimethoxyphenyl)oxymethyl]-2-phenylcyclopropanecarboxamide,
2) N-methyl-N-phenyl-2-[(3,4-dimethoxyphenyl)oxymethyl]-2-phenylcyclopropanecarboxamide,
3) N-(5-fluoropyridin-2-yl)-2-[(3,4-dimethoxyphenyl)oxymethyl]-2-phenylcyclopropanecarboxamide,
4) (1R,2S)—N-(5-cyanopyridin-2-yl)-2-[(3,4-dimethoxyphenyl)oxymethyl]-2-phenylcyclopropanecarboxamide,
5) N-(4-methyl-1,3-thiazol-2-yl)-2-[(3,4-dimethoxyphenyl)oxymethyl]-2-phenylcyclopropanecarboxamide,
6) N-(5-chloropyridin-2-yl)-2-[(3,4-dimethoxyphenyl)oxymethyl]-2-phenylcyclopropanecarboxamide,
7) N-(3-methyl-1,2,4-thiadiazol-5-yl)-2-[(3,4-dimethoxyphenyl)oxymethyl]-2-phenylcyclopropanecarboxamide,
8) N-(3-methylisoxazol-5-yl)-2-[(3,4-dimethoxyphenyl)oxymethyl]-2-phenylcyclopropanecarboxamide,
9) N-(1,5-dimethyl-1H-pyrazol-3-yl)-2-[(3,4-dimethoxyphenyl)oxymethyl]-2-phenylcyclopropanecarboxamide,
10) N-(5-fluoropyridin-2-yl)-2-[(3,4-dimethoxyphenyl)oxymethyl]-2-phenylcyclopropanecarboxamide,
11) N-(5-chloropyridin-3-yl)-2-[(3,4-dimethoxyphenyl)oxymethyl]-2-phenylcyclopropanecarboxamide,
12) N-(2-methylpyridin-4-yl)-2-[(3,4-dimethoxyphenyl)oxymethyl]-2-phenylcyclopropanecarboxamide,
13) N-(5-chloro-1,3-thiazol-2-yl)-2-[3,4-dimethoxyphenyl)oxymethyl]-2-phenylcyclopropanecarboxamide,
14) N-(3-chloropyridin-4-yl)-2-[(3,4-dimethoxyphenyl)oxymethyl]-2-phenylcyclopropanecarboxamide,
15) N-(5-methylpyridin-3-yl)-2-[(3,4-dimethoxyphenyl)oxymethyl]-2-phenylcyclopropanecarboxamide,
16) N-(1,3,4-thiadiazol-2-yl)-2-[(3,4-dimethoxyphenyl)oxymethyl]-2-phenylcyclopropanecarboxamide,
17) N-(5-methyl-1,3,4-thiadiazol-2-yl)-2-[(3,4-dimethoxyphenyl)oxymethyl]-2-phenylcyclopropanecarboxamide,
18) N-(4-cyanopyridin-2-yl)-2-[(3,4-dimethoxyphenyl)oxymethyl]-2-phenylcyclopropanecarboxamide,
19) N-(2-fluoropyridin-4-yl)-2-[(3,4-dimethoxyphenyl)oxymethyl]-2-phenylcyclopropanecarboxamide,
20) N-(3-methylpyridin-4-yl)-2-[(3,4-dimethoxyphenyl)oxymethyl]-2-phenylcyclopropanecarboxamide,
21) N-(5-methylisoxazol-3-yl)-2-[(3,4-dimethoxyphenyl)oxymethyl]-2-phenylcyclopropanecarboxamide,
22) N-(5-methyl-1,3-thiazol-2-yl)-2-[(3,4-dimethoxyphenyl)oxymethyl]-2-phenylcyclopropanecarboxamide,
23) N-(4-methyl-1,3-thiazol-2-yl)-2-[(3,4-dimethoxyphenyl)oxymethyl]-2-phenylcyclopropanecarboxamide,
24) N-(5-fluoropyridin-3-yl)-2-[(3,4-dimethoxyphenyl)oxymethyl]-2-phenylcyclopropanecarboxamide,
25) N-(3-trifluoromethylpyridin-4-yl)-2-[(3,4-dimethoxyphenyl)oxymethyl]-2-phenylcyclopropanecarboxamide,
26) N-(6-fluoromethylpyridin-3-yl)-2-[(3,4-dimethoxyphenyl)oxymethyl]-2-phenylcyclopropanecarboxamide,
27) N-(6-fluoropyridin-2-yl)-2-[(3,4-dimethoxyphenyl)oxymethyl]-2-phenylcyclopropanecarboxamide,
28) N-(4-chloropyridin-2-yl)-2-[(3,4-dimethoxyphenyl)oxymethyl]-2-phenylcyclopropanecarboxamide,
29) N-(5-cyanopyridin-2-yl)-2-[(3,4-dimethoxyphenyl)oxymethyl]-2-phenylcyclopropanecarboxamide,
30) N-(6-chloropyridazin-3-yl)-2-[(3,4-dimethoxyphenyl)oxymethyl]-2-phenylcyclopropanecarboxamide,
31) N-(6-cyanopyridin-3-yl)-2-[(3,4-dimethoxyphenyl)oxymethyl]-2-phenylcyclopropanecarboxamide,
32) N-(2-chloropyridin-4-yl)-2-[(3,4-dimethoxyphenyl)oxymethyl]-2-phenylcyclopropanecarboxamide,
33) N-(pyrimidin-4-yl)-2-[(3,4-dimethoxyphenyl)oxymethyl]-2-phenylcyclopropanecarboxamide,
34) N-(3-methoxyphenyl)-2-[(3,4-dimethoxyphenyl)oxymethyl]-2-phenylcyclopropanecarboxamide,
35) N-[2-(1H-1,2,4-triazol-3-yl)phenyl]-2-[(3,4-dimethoxyphenyl)oxymethyl]-2-phenylcyclopropanecarboxamide,
36) N-(4-methylpyridin-2-yl)-2-[(3,4-dimethoxyphenyl)oxymethyl]-2-phenylcyclopropanecarboxamide,
37) N-(6-methylpyridin-2-yl)-2-[(3,4-dimethoxyphenyl)oxymethyl]-2-phenylcyclopropanecarboxamide,
38) N-(6-chloropyridin-2-yl)-2-[(3,4-dimethoxyphenyl)oxymethyl]-2-phenylcyclopropanecarboxamide,
39) N-(1-ethyl-1H-pyrazol-5-yl)-2-[(3,4-dimethoxyphenyl)oxymethyl]-2-phenylcyclopropanecarboxamide,
40) N-(3-methylisothiazol-5-yl)-2-[(3,4-dimethoxyphenyl)oxymethyl]-2-phenylcyclopropanecarboxamide,
41) (1R,2S)—N-(pyridin-2-yl)-2-[(3,4-dimethoxyphenyl)oxymethyl]-2-phenylcyclopropanecarboxamide,
42) N-(pyridin-3-yl)-2-[(3,4-dimethoxyphenyl)oxymethyl]-2-phenylcyclopropanecarboxamide,
43) N-(pyridin-4-yl)-2-[(3,4-dimethoxyphenyl)oxymethyl]-2-phenylcyclopropanecarboxamide,
44) 2-{[(3,4-dimethoxyphenoxy)methyl]-2-phenylcyclopropyl}-5-fluoro-1H-benzimidazole,
45) 3-{2-[(3,4-dimethoxyphenoxy)methyl]-2-phenylcyclopropyl}-5-phenyl-1H-1,2,4-triazole,
46) N-(pyridin-2-yl)-2-(3-methoxyphenyloxymethyl)-2-phenylcyclopropanecarboxamide,
47) N-(pyridin-2-yl)-2-(4-methoxyphenyloxymethyl)-2-phenylcyclopropanecarboxamide,
48) N-(pyridin-2-yl)-2-[(3-methoxy-4-methoxymethylphenyl)oxymethyl]-2-phenylcyclopropanecarboxamide,
49) N-(pyridin-2-yl)-2-(3,4-dimethoxyphenyl)-2-phenyloxymethylcyclopropanecarboxamide,
50) N-(pyridin-2-yl)-2-[(4-methoxy-3-methoxymethylphenyl)oxymethyl]-2-phenylcyclopropanecarboxamide,
51) N-(pyridin-2-yl)-2-(3,4-dimethoxyphenyl)-2-(4-fluorophenyloxymethyl)cyclopropanecarboxamide,
52) N-(5-fluoropyridin-2-yl)-2-(3,4-dimethoxyphenyl)-2-(4-fluorophenyloxymethyl)cyclopropanecarboxamide,
53) N-(5-chloropyridin-2-yl)-2-[(1-methyl-6-oxo-1,6-dihydropyridin-3-yl)oxymethyl]-2-phenylcyclopropanecarboxamide,
54) N-(5-chloropyridin-2-yl)-2-[(1-ethyl-6-oxo-1,6-dihydropyridin-3-yl)oxymethyl]-2-phenylcyclopropanecarboxamide,
55) N-(5-fluoropyridin-2-yl)-2-[(1-ethyl-6-oxo-1,6-dihydropyridin-3-yl)oxymethyl]-2-phenylcyclopropanecarboxamide,
56) N-(pyridin-2-yl)-2-[(3,4-dimethoxyphenyl)oxymethyl]-2-(pyridin-2-yl)cyclopropanecarboxamide,
57) (1S,2R)—N-(5-cyanopyridin-2-yl)-2-[(1-ethyl-3-methyl-1H-pyrazol-4-yl)oxymethyl]-2-phenylcyclopropanecarboxamide,
58) (1S,2R)—N-(pyridin-2-yl)-2-[(1-ethyl-3-methyl-1H-pyrazol-4-yl)oxymethyl]-2-phenylcyclopropanecarboxamide,
59) (1S,2R)—N-(6-fluoropyridin-3-yl)-2-[(1-ethyl-3-methyl-1H-pyrazol-4-yl)oxymethyl]-2-phenylcyclopropanecarboxamide,
60) (1S,2R)—N-(4-fluorophenyl)-2-[(1-ethyl-3-methyl-1H-pyrazol-4-yl)oxymethyl]-2-phenylcyclopropanecarboxamide,
61) (1S,2R)—N-(5-methoxypyridin-3-yl)-2-[(1-ethyl-3-methyl-1H-pyrazol-4-yl)oxymethyl]-2-phenylcyclopropanecarboxamide,
62) (1S,2R)—N-(2-fluoropyridin-4-yl)-2-[(1-ethyl-3-methyl-1H-pyrazol-4-yl)oxymethyl]-2-phenylcyclopropanecarboxamide,
63) N-(5-cyanopyridin-2-yl)-2-[(1-ethyl-1H-pyrazol-4-yl)oxymethyl]-2-phenylcyclopropanecarboxamide,
64) (1R,2S)-2-[(1-ethyl-3-methyl-1H-pyrazol-4-yl)oxymethyl]-2-(3-fluorophenyl)-N-(pyridin-2-yl)cyclopropanecarboxamide,
65) (1R,2S)-2-[(1-ethyl-3-methyl-1H-pyrazol-4-yl)oxymethyl]-2-(3-fluorophenyl)-N-(5-fluoropyridin-2-yl)cyclopropanecarboxamide,
66) (1R,2S)-2-[(1-ethyl-3-methyl-1H-pyrazol-4-yl)oxymethyl]-2-(3-fluorophenyl)-N-(5-fluoropyridin-3-yl)cyclopropanecarboxamide,
67) (1R,2S)-2-[(1-ethyl-3-methyl-1H-pyrazol-4-yl)oxymethyl]-2-(3-fluorophenyl)-N-(4-fluorophenyl)cyclopropanecarboxamide,
68) (1R,2S)-2-[(1-ethyl-3-methyl-1H-pyrazol-4-yl)oxymethyl]-N-(5-fluoro-4-methoxypyridin-2-yl)-2-(3-fluorophenyl)cyclopropanecarboxamide,
69) (1R,2S)—N-(5-cyanopyridin-2-yl)-2-[(1-ethyl-3-methyl-1H-pyrazol-4-yl)oxymethyl]-2-(3-fluorophenyl)cyclopropanecarboxamide,
70) (1R,2S)-2-[(1-ethyl-3-methyl-1H-pyrazol-4-yl)oxymethyl]-2-(3-fluorophenyl)-N-(2-fluoropyridin-4-yl)cyclopropanecarboxamide,
71) (1R,2S)-2-[(1,3-dimethyl-6-oxo-1,6-dihydropyridazin-4-yl)oxymethyl]-N-(5-methoxypyridin-3-yl)-2-phenylcyclopropanecarboxamide,
72) (1R,2S)-2-[(1,3-dimethyl-6-oxo-1,6-dihydropyridazin-4-yl)oxymethyl]-N-(4-fluorophenyl)-2-phenylcyclopropanecarboxamide,
73) (1R,2S)-2-[(1,3-dimethyl-6-oxo-1,6-dihydropyridazin-4-yl)oxymethyl]-N-(5-fluoro-4-methylpyridin-3-yl)-2-phenylcyclopropanecarboxamide,
74) (1R,2S)—N-(5-chloropyridin-3-yl)-2-[(1,3-dimethyl-6-oxo-1,6-dihydropyridazin-4-yl)oxymethyl]-2-phenylcyclopropanecarboxamide,
75) (1R,25)-2-[2-(3-difluoromethyl-5-methyl-1H-1,2,4-triazol-1-yl)ethyl]-N-(5-fluoro-4-methylpyridin-2-yl)-2-phenylcyclopropanecarboxamide,
76) (1R,2S)-2-[2-(3-difluoromethyl-5-methyl-1H-1,2,4-triazol-1-yl)ethyl]-N-(5-fluoropyridin-2-yl)-2-phenylcyclopropanecarboxamide,
77) (1R,2S)—N-(5-chloropyridin-2-yl)-2-[2-(3-difluoromethyl-5-methyl-1H-1,2,4-triazol-1-yl)ethyl]-2-phenylcyclopropanecarboxamide,
78) (1R,2S)-2-[2-(3-difluoromethyl-5-methyl-1H-1,2,4-triazol-1-yl)ethyl]-N-(4-fluorophenyl)-2-phenylcyclopropanecarboxamide,
79) (1R,2S)—N-(5-fluoropyridin-2-yl)-2-[2-(3-difluoromethyl-5-methyl-1H-1,2,4-triazol-1-yl)ethyl]-2-phenylcyclopropanecarboxamide,
80) N-(5-fluoropyridin-2-yl)-2-[2-(3-methoxy-4-oxopyridine-1(4H)-yl)ethyl]-2-phenylcyclopropanecarboxamide,
81) 2-[2-(3,5-dimethyl-1H-1,2,4-triazol-1-yl)ethyl]-N-(5-fluoropyridin-2-yl)-2-phenylcyclopropanecarboxamide,
82) N-(5-chloropyridin-2-yl)-2-[2-(3,5-dimethyl-1H-1,2,4-triazol-1-yl)ethyl]-2-phenylcyclopropanecarboxamide,
83) (1R,2S)—N-(5-fluoropyridin-2-yl)-2-[2-(5-methyl-3-trifluoromethyl-1H-1,2,4-triazol-1-yl)ethyl]-2-phenylcyclopropanecarboxamide,
84) (1R,2S)—N-(5-chloropyridin-2-yl)-2-[2-(5-methyl-3-trifluoromethyl-1H-1,2,4-triazol-1-yl)ethyl]-2-phenylcyclopropanecarboxamide,
85) (1R,2S)—N-(5-cyanopyridin-2-yl)-2-[2-(5-methyl-3-trifluoromethyl-1H-1,2,4-triazol-1-yl)ethyl]-2-phenylcyclopropanecarboxamide,
86) (1R,2S)—N-(5-fluoropyridin-2-yl)-2-[2-(3-cyclopropyl-5-methyl-1H-1,2,4-triazol-1-yl)ethyl]-2-phenylcyclopropanecarboxamide,
87) (1R,2S)—N-(5-chloropyridin-2-yl)-2-[2-(3-cyclopropyl-5-methyl-1H-1,2,4-triazol-1-yl)ethyl]-2-phenylcyclopropanecarboxamide,
88) (1R,2S)—N-(5-cyanopyridin-2-yl)-2-[2-(3-cyclopropyl-5-methyl-1H-1,2,4-triazol-1-yl)ethyl]-2-phenylcyclopropanecarboxamide,
89) (1R,2S)—N-(5-fluoropyridin-2-yl)-2-[2-(3-ethyl-5-methyl-1H-1,2,4-triazol-1-yl)ethyl]-2-phenylcyclopropanecarboxamide, and
90) (1R,2S)—N-(5-chloropyridin-2-yl)-2-[2-(3-ethyl-5-methyl-1H-1,2,4-triazol-1-yl)ethyl]-2-phenylcyclopropanecarboxamide,
or a pharmaceutically acceptable salt thereof.

13. A pharmaceutical composition comprising, as an active ingredient, the compound according to any one of claims 1 to 12 or a pharmaceutically acceptable salt thereof.

14-15. (canceled)

16. A method for treating sleep disorder for which orexin receptor antagonism is effective, which comprises administering the compound according to any one of claims 1 to 12 or a pharmaceutically acceptable salt thereof into a subject in need thereof.

17. The method according to claim 16, wherein said sleep disorder is insomnia.
